(19) United States
(12) Patent Application Publication
(10) Pub. No.: US 2024/0199561 A1
(43) Pub. Date: Jun. 20, 2024

(54) INHIBITORS OF DYRK AND PIM

(71) Applicants: COLD SPRING HARBOR LABORATORY, Cold Spring Harbor, NY (US); THE FEINSTEIN INSTITUTES FOR MEDICAL RESEARCH, Manhasset, NY (US); VICHEM CHEMIE RESEARCH, LTD., Budapest (HU)

(72) Inventors: Nicholas TONKS, Cold Spring Harbor, NY (US); Carla-Maria GAUSS, Cold Spring Harbor, NY (US); Prabhadevi VENKATARAMANI, Cold Spring Harbor, NY (US); Leemor JOSHUA-TOR, Cold Spring Harbor, NY (US); Elad ELKAYAM, Cold Spring Harbor, NY (US); Yousef Al ABED, Manhassett, NY (US); Kai Fan CHENG, Manhassett, NY (US); Ahmad ALTITI, Manhassett, NY (US); Laszlo ORFI, Budapest (HU); Istvan SZABADKAI, Budapest (HU); Balint SZOKOL, Budapest (HU); Zoltan HORVATH, Budapest (HU)

(73) Assignees: COLD SPRING HARBOR LABORATORY, Cold Spring Harbor, NY (US); VICHEM CHEMIE RESEARCH, LTD., Budapest (HU); THE FEINSTEIN INSTITUTES FOR MEDICAL RESEARCH, Manhasset, NY (US)

(21) Appl. No.: 18/261,563

(22) PCT Filed: Jan. 19, 2022
(86) PCT No.: PCT/US2022/012894

§ 371 Date: Jul. 14, 2023

Related U.S. Application Data
(60) Provisional application No. 63/139,112, filed on Jan. 19, 2021.

(51) Int. Cl. C07D 277/66; A61K 31/428; A61K 31/431; A61K 31/506; C07D 513/04

(52) U.S. Cl. CPC C07D 277/66; A61K 31/428; A61K 31/431; A61K 31/506; C07D 513/04

(57) ABSTRACT

Provided is a compound, including a compound of Formula I:

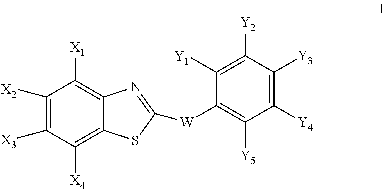

or a pharmaceutically acceptable salt thereof, wherein W is a direct bond or an optionally substituted C, and if W is a direct bond, then X₁, X₂, X₃, and X₄ are each independently H, OH, or an electrophile, and optionally one pair selected from X₁ and X₂, X₂ and X₃, and X₃ and X₄ forms a five-membered ring including one or more heteroatom, wherein the one or more heteroatom is selected from O, N, and S, and the five-membered ring is optionally substituted with a ═C, an ═S, or an electrophile, and Y₁, Y₂, Y₃, Y₄ and Y₅ are each independently selected from H, CH₃, OH, O—CH₃, and NO₂, and an electrophile, and optionally Y₂ and Y₃ form a five-membered ring including one or more heteroatom, wherein the one or more heteroatom is selected from O, N, and S, and the five-membered ring is optionally substituted with a ═C, ═S, or an electrophile.

### CROSS-REFERENCE TO RELATED APPLICATIONS

This application claims benefit of priority from U.S. Provisional Patent Application No. 63/139,112, filed Jan. 19, 2021, the entire contents of which are incorporated herein by reference.

### BACKGROUND

Dual-specificity tyrosine (Y)-phosphorylation-regulated kinases (DYRKs) DYRKs belong to the CMGC family of serine/threonine kinases (S/T kinases), a well-conserved family of kinases that includes cyclin-dependent kinases (CDKs), mitogen-activated protein kinases (MAP kinases), glycogen synthase kinases (GSK) and CDK-like kinases. DYRKs catalyze self-activation through autophosphorylation of a single Tyr residue in their activation loop and catalyze phosphorylation of serine (S) and threonine (T) residues in exogenous protein substrates. Highly conserved across species, DYRKs show little sequence homology to other kinases outside of their catalytic domains.

FIG. 1 shows structural comparisons of various DYRK isoforms. The gene for the DYRK isoform DYRK1A lies within the Down Syndrome (DS) Critical Region on chromosome 21 (21q22.13), also known as trisomy 21, and is 1.5-fold upregulated in brains of subjects with DS. DS patients have many abnormalities including cognitive impairments such as intellectual disability, deficits in learning and memory, and early onset Alzheimer's disease (AD). Overexpression of DYRK1A in brains of subjects with DS may contribute to early onset of AD pathology through the hyperphosphorylation of Tau protein (T212, S202 and S404), and subjects with AD have cognitive or affective impairments including deficits in memory, recognition of people or places, mood changes, and difficulty exercising judgment and planning. FIG. 2A shows a ribbon diagram of DYRK1A. FIG. 2B. identifies some features of the DYRK1A kinase domain. The Tyr autophosphorylation site is located ˜20 bp upstream of the SPE motif between subdomains VII and VIII (Tyr 321). FIG. 2C identifies some features of DYRK1A's substrate specificity.

DYRK1A is also a potential therapeutic target in many cancers. For example, acute megakaryoblastic leukemia (AMKL) is more frequently observed in DS, and increased DYRK1A expression can promote leukemia in a murine model of DS. Furthermore, epidermal growth factor receptor (EGFR) is one of the most prevalent genes altered and/or amplified in approx. 50% of primary tumors in glioblastomas, and DYRK1A is overexpressed in a subset of gliomas, especially ones that contain high levels of EGFR. In turn, blocking DYRK1A kinase activity impairs tumor growth in EGFR-dependent sensitive lines. Although several small-molecule inhibitors of DYRK1A have been identified, they suffer from one or more various shortcomings, including disadvantageous side effects including hallucinogenic and psychoactive effects and cardiac complications, low potency, low selectivity. A potent and selective inhibitor of DYRK1A is therefore desirable.

Recent studies have shown that certain non-small cell lung cancer (NSCLC) patients show high DYRK1A expression levels which was associated with poor prognosis. Inhibition of DYRK1A or its siRNA knockdown impairs cell proliferation as well as results in low EGFR levels in NSCLC cells with wild-type EGFR. Additionally, DYRK1A inhibition with harmine was found to sensitize cells to the EGFR inhibitor AZD9291. Thus, DYRK1A might also be a novel therapeutic target in NSCLC and combination therapy using DYRK and EGFR inhibitors could be beneficial to these patients.

Proviral integration site for Moloney murine leukemia virus proteins (PIMs) belong to the CAMK (calmodulin-dependent protein kinase-related) group of protein kinases. PIMs are constitutively active with a short half-life. Heat shock protein (Hsp90) mediates protection of PIMs from proteasomal degradation. The PIM isoform PIM1 is a therapeutic target in many cancers, found to be highly expressed in leukemia, lymphoma, prostate, pancreatic and triple-negative breast cancer. Small-molecule inhibitors of PIM1 have been identified, though they suffer to varying degrees from one or more shortcomings, including a narrow potential therapeutic dose range, low selectivity, and low potency. FIG. 3A shows a ribbon diagram of PIM1. FIG. 3B. identifies some structural features and substrate specificity of the DYRK1A kinase domain. PIM1 strongly prefers basic residues, particularly arginine, at positions P−5 and P−3. PIM1 also prefers histidine at P−2, proline at P−1 and glycine at P+1 positions. Aligning amino acid sequences of PIM1 and DYRK1A identifies amino acids that contribute to potential interactions of DYRK1A and PIM1 inhibitors in the ATP-binding domain of DYRK1A and PIM1. A potent and selective inhibitor of PIM1 is therefore desirable.

### SUMMARY

The present disclosure is directed to overcoming these and other deficiencies in the art. In an aspect, disclosed is a compound, including a compound of Formula I:

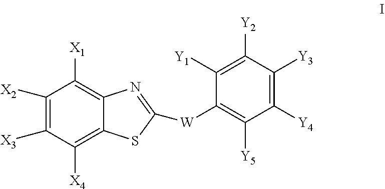

or a pharmaceutically acceptable salt thereof, wherein W is a direct bond or an optionally substituted C, and
if W is a direct bond, then X₁, X₂, X₃, and X₄ are each independently H, OH, or an electrophile, and optionally one pair selected from X₁ and X₂, X₂ and X₃, and X₃ and X₄ forms a five-membered ring including one or more heteroatom, wherein the one or more heteroatom is selected from O, N, and S, and the five-membered ring is optionally substituted with a ═C, an ═S, or an electrophile, and
Y₁, Y₂, Y₃, Y₄ and Y₅ are each independently selected from H, CH₃, OH, O—CH₃, and NO₂, and an electrophile, and optionally Y₂ and Y₃ form a five-membered ring including one or more heteroatom, wherein the one or more heteroatom is selected from O, N, and S, and the five-membered ring is optionally substituted with a ═C, ═S, or an electrophile,
with the caveats that
(a) if the pair X₂ and X₃ together form a dioxolane then optionally Y₂ and Y₃ form a five-membered ring including one or more heteroatom only if Y₂ and Y₃ form a pyrroline optionally substituted with a ═C, ═S, or an electrophile, and no more than one of Y₁, Y₂, Y₃, Y₄ and Y₅ is OH,
(b) if one or more of Y₁, Y₂, Y₃, Y₄ and Y₅ is O—CH₃, then X₂ and X₃ form a five-membered ring including one or more heteroatom, wherein the one or more heteroatom is selected from N and S, and the five-membered ring is optionally substituted with a ═C, ═S, or an electrophile, and
(c) if X₃ is OH and one or both of Y₃ and Y₄ are OH then Y₅ is OH; and
(d) if W is an optionally substituted C, then
   (i) at least one of X₁, X₂, X₃, X₄ is independently selected from OH, O—CH₃, and a halogen, at least one of Y₁, Y₂, Y₃, Y₄, and Y₅ is independently selected from OH, O—CH₃, and a halogen, no more than five of X₁, X₂, X₃, X₄, Y₁, Y₂, Y₃, Y₄, and Y₅ is H, and no more than one of X₁, X₂, X₃, X₄, Y₁, Y₂, Y₃, Y₄, and Y₅, is O—CH₃, or
   (ii) X₁, X₄, Y₁, Y₄, and Y₅ are hydrogen, X₂ and X₃ are OH or together form a dioxolane, and Y₂ and Y₃ are OH or together form a dioxolane.

In an example, the pair X₂ and X₃ together form a dioxolane and Y₂ and Y₃ form a pyrrolidine optionally substituted with a ═C, ═S, or an electrophile, and no more than one of Y₁, Y₂, Y₃, Y₄ and Y₅ is OH.

In another example, one or more of Y₁, Y₂, Y₃, Y₄ and Y₅ is O—CH₃, and X₂ and X form a five-membered ring including one or more heteroatom, wherein the one or more heteroatom is selected from N and S, and the five-membered ring is optionally substituted with a ═C, ═S, or an electrophile.

In another example, X₃ is OH and one or both of Y₃ and Y₄ are OH and Y₅ is OH.

In another example, W is an optionally substituted C and at least one of X₁, X₂, X₃, X₄ is independently selected from OH, O—CH₃, and a halogen, at least one of Y₁, Y₂, Y₃, Y₄, and Y₅ is independently selected from OH, O—CH₃, and a halogen, no more than five of X₁, X₂, X₃, X₄, Y₁, Y₂, Y₃, Y₄, and Y₅ is H, and no more than one of X₁, X₂, X₃, X₄, Y₁, Y₂, Y₃, Y₄, and Y₅, is O—CH₃.

In another example, W is an optionally substituted C and X₁, X₄, Y₁, Y₄, and Y₅ are hydrogen, X₂ and X₃ are OH or together form a dioxolane, and Y₂ and Y₃ are OH or together form a dioxolane.

In an example, W is an optionally substituted C. In another example, W comprises —C(—O)—. In another example, the compound includes Formula Ia:

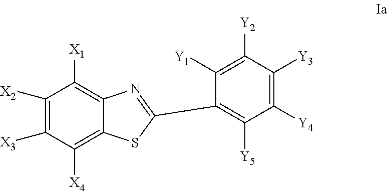

or a pharmaceutically acceptable salt thereof.

In another aspect, disclosed in a pharmaceutical composition, including any one of the foregoing compound of Formula I, or a pharmaceutically acceptable salt thereof, and a pharmaceutically acceptable excipient. In an example, the compound included in the pharmaceutical composition includes any one of the foregoing compound of Formula Ia or pharmaceutically acceptable salt thereof.

In yet another aspect, provided is method including administering any one or more of the foregoing pharmaceutical compositions to a subject, wherein the subject is diagnosed with or at risk of developing cancer. An example further includes administering an epidermal growth factor receptor (EGFR) inhibitor in combination with the pharmaceutical composition, or wherein the pharmaceutical composition further includes the EGFR inhibitor. In another example, the EGFR inhibitor includes AZD9291. In yet another example, the cancer is selected from acute megakaryoplastic leukemia, glioblastoma, and non-small cell lung cancer

In still another aspect, provided is method including treating a cognitive impairment in a subject, wherein the treating comprises administering any one or more of the foregoing pharmaceutical compositions to the subject. In an example, the subject has Down syndrome, or trisomy 21. In another example, the subject is diagnosed with or at risk for developing Alzheimer's disease. In another example, provided is a method including administering any one or more of the foregoing pharmaceutical compositions to a subject, wherein the subject is diagnosed with Down syndrome, or trisomy 21. In a further aspect, provided is method including administering any one or more of the foregoing pharmaceutical compositions to a subject, wherein the subject is diagnosed with Alzheimer's disease.

In another aspect, disclosed is a compound including Formula I:

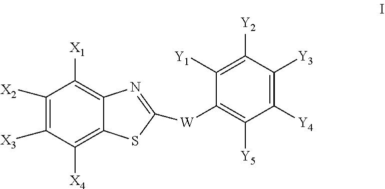

or a pharmaceutically acceptable salt thereof, wherein
W is an optionally substituted C,
at least one of X₁, X₂, X₃, X₄ is independently selected from OH, O—CH₃, and a halogen,
Y₁, Y₂, Y₃, Y₄ and Y₅ are each independently selected from H, CH₃, OH, O—CH₃, NO₂, and an electrophile, at least one of Y₁, Y₂, Y₃, Y₄, and Y₅ is independently selected from OH and O—CH₃, and a halogen, and optionally Y₂ and Y₃ form a five-membered ring including one or more heteroatom, wherein the one or more heteroatom is selected from O, N, and S, and the five-membered ring is optionally substituted with a ═C, ═S, or an electrophile, and
no more than five of X₁, X₂, X₃, X₄, Y₁, Y₂, Y₃, Y₄, and Y₅ is H, and no more than one of X₁, X₂, X₃, X₄, Y₁, Y₂, Y₃, Y₄, and Y₅, is O—CH₃.

In an example, W comprises —C(═O)—. In another example, the compound is selected from

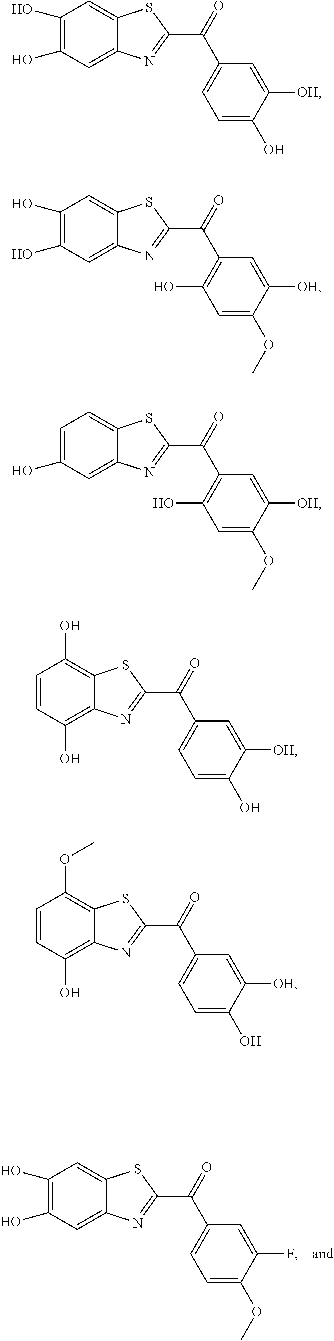 [SMILES: O=C(c1ccc(O)c(O)c1)c1nc2cc(O)c(O)cc2s1 ; COc1cc(O)c(C(=O)c2nc3cc(O)c(O)cc3s2)cc1O ; COc1cc(O)c(C(=O)c2nc3cc(O)ccc3s2)cc1O ; O=C(c1ccc(O)c(O)c1)c1nc2c(O)ccc(O)c2s1 ; COc1ccc(O)c2nc(C(=O)c3ccc(O)c(O)c3)sc12 ; COc1ccc(C(=O)c2nc3cc(O)c(O)cc3s2)cc1F]

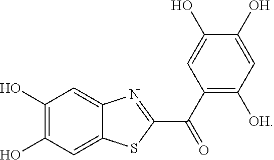 [SMILES: O=C(c1nc2cc(O)c(O)cc2s1)c1cc(O)c(O)cc1O]

In yet another example, the electrophile is selected from

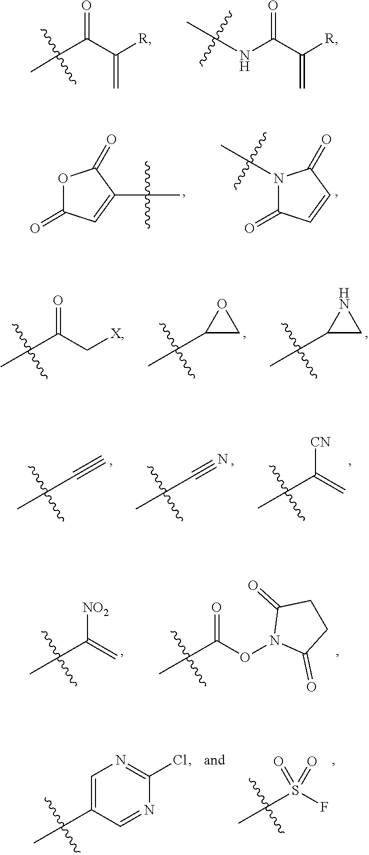

wherein R═H or any alkyl or any carbonyl, and X=any halogen, ether, amine, thiol, or thioether.

In still another example, the compound is not substituted with an electrophile.

In still a further example, disclosed is a pharmaceutical composition including any of the foregoing compounds and a pharmaceutically acceptable excipient.

In still another aspect, disclosed is a compound including Formula I:

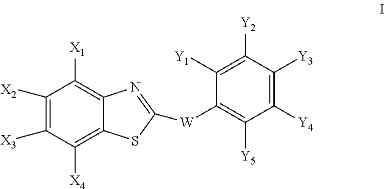

or a pharmaceutically acceptable salt thereof, wherein
W is an optionally substituted C, and
X₁, X₄, Y₁, Y₄, and Y₅ are hydrogen, X₂ and X₃ are OH or together form a dioxolane, and Y₂ and Y₃ are OH or together form a dioxolane. In an example, W includes —C(═O)—.

In another example, the compound is

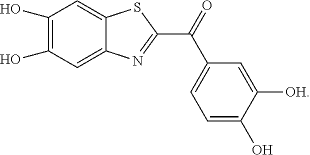 [SMILES: O=C(c1ccc(O)c(O)c1)c1nc2cc(O)c(O)cc2s1]

In still another example, provided is a pharmaceutical composition, including any of the foregoing compounds of Formula I or a pharmaceutically acceptable salt thereof and a pharmaceutically acceptable excipient.

In yet another example, disclosed is a compound, including Formula Ia:

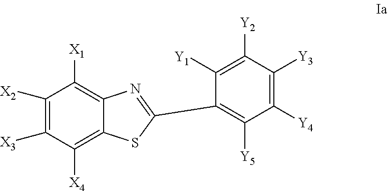

or a pharmaceutically acceptable salt thereof, wherein
X₁, X₂, X₃, and X₄ are each independently H, OH, or an electrophile, and optionally one pair selected from X₁ and X₂, X₂ and X₃, and X₃ and X₄ forms a five-membered ring including one or more heteroatom, wherein the one or more heteroatom is selected from O, N, and S, and the five-membered ring is optionally substituted with a ═C, an ═S, or an electrophile, and
Y₁, Y₂, Y₃, Y₄ and Y₅ are each independently selected from H, CH₃, OH, O—CH₃, NO₂, and an electrophile, and optionally Y₂ and Y₃ form a five-membered ring including one or more heteroatom, wherein the one or more heteroatom is selected from O, N, and S, and the five-membered ring is optionally substituted with a ═C, ═S, or an electrophile,
with the caveats that
(a) if the pair X₂ and X₃ together form a dioxolane then optionally Y₂ and Y₃ form a pyrroline optionally substituted with a ═C, ═S, or an electrophile, and no more than one of Y₁, Y₂, Y₃, Y₄ and Y₅ is OH,
(b) if one or more of Y₁, Y₂, Y₃, Y₄ and Y₅ is O—CH₃, then X₂ and X₃ form a five-membered ring including one or more heteroatom, wherein the one or more heteroatom is selected from N and S, and the five-membered ring is optionally substituted with a ═C, ═S, or an electrophile, and
(c) if X₃ is OH and one or both of Y₃ and Y₄ are OH then Y₅ is OH.

In an example, X₁ and X₂, or X₃ and X₄, form an imidazole or a triazole. In another example, X₂ and X₃ form a five-membered ring selected from a pyrrole, a dioxolane, a pyrazole, an imidazole, and a triazole. In yet another example, X₂ and X₃ form a five-membered ring including a substitution, the five-membered ring is selected from a pyrroline and a tetrahydrofuran, and the substitution is selected from ═O, ═S, and an electrophile. In still another example, X₂ and X₃ form a thiazole including a substitution, and the substitution is selected from ═O, ═S, and an electrophile. In a further example, X₂ and X₃ form a dioxolane. In yet a further example, Y₂ and Y₃ form a five-membered ring. In still a further example, Y₂ and Y₃ form a furan, a pyrrole, or a thiophene. In another example, Y₂ and Y₃ form a five-membered ring including a substitution, wherein the five-membered ring is selected from a pyrroline and a thiazole, and the substitution is selected from ═O, ═S, and an electrophile.

In yet another example, the compound is selected from

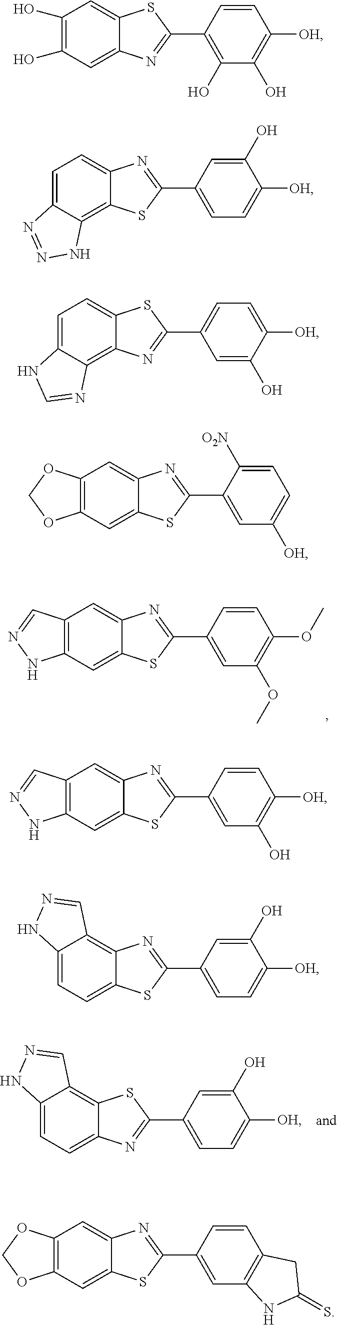

In still another example, the electrophile is selected from

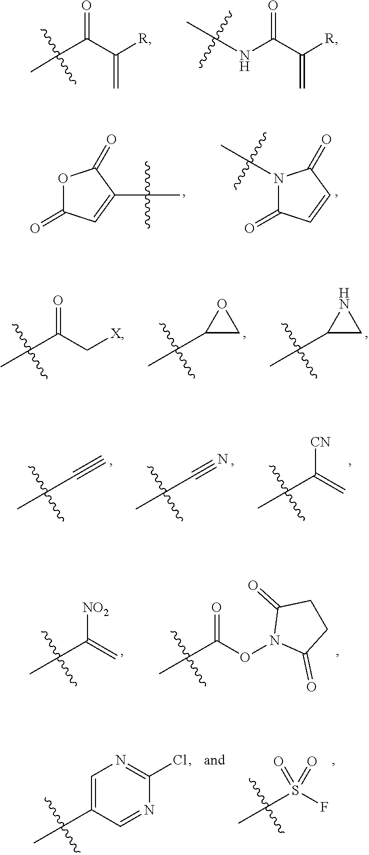

wherein R═H or any alkyl or any carbonyl, and X=any halogen, ether, amine, thiol, or thioether.

In a further example, the compound is not substituted with an electrophile.

Also disclosed is a pharmaceutical composition, including any of the foregoing compounds of Formula Ia or a pharmaceutical salt thereof and a pharmaceutically acceptable excipient.

In still another aspect, disclosed is a compound, including Formula Ia:

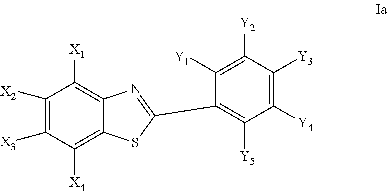

or a pharmaceutically acceptable salt thereof, wherein
X₁, X₂, X₃, and X₄ are each independently H, OH, or an electrophile, and optionally one pair selected from X₁ and X₂, X₂ and X₃, and X₃ and X₄ forms a five-membered ring including one or more heteroatom, wherein the one or more heteroatom is selected from O, N, and S, and the five-membered ring is not a dioxolane and is optionally substituted with a ═C, an ═S, or an electrophile, and
Y₁, Y₂, Y₃, Y₄ and Y₅ are each independently selected from H, CH₃, OH, O—CH₃, NO₂, and an electrophile, and optionally Y₂ and Y₃ form a five-membered ring including one or more heteroatom, wherein the one or more heteroatom is selected from O, N, and S, and the five-membered ring is optionally substituted with a ═C, ═S, or an electrophile,
with the caveats that
(a) if one or more of Y₁, Y₂, Y₃, Y₄ and Y₅ is O—CH₃, then X₂ and X₃ form a five-membered ring including one or more heteroatom, wherein the one or more heteroatom is selected from N and S, and the five-membered ring is optionally substituted with a ═C, ═S, or an electrophile, and
(b) if X₃ is OH and one or both of Y₃ and Y₄ are OH then Y₅ is OH.

In an example, X₁ and X₂, or X₃ and X₄, form an imidazole or a triazole. In another example, X₂ and X₃ form a five-membered ring selected from a pyrrole, a dioxolane, a pyrazole, an imidazole, and a triazole. In still another example, X₂ and X₃ form a five-membered ring including a substitution, the five-membered ring is selected from a pyrroline and a tetrahydrofuran, and the substitution is selected from ═O, ═S, and an electrophile. In a further example, X₂ and X₃ form a thiazole including a substitution, and the substitution is selected from ═O, ═S, and an electrophile. In yet a further example, X₂ and X₃ form a dioxolane. In still a further example, Y₂ and Y₃ form a five-membered ring.

In another example, Y₂ and Y₃ form a furan, a pyrrole, or a thiophene. In still another example, Y₂ and Y₃ form a five-membered ring including a substitution, wherein the five-membered ring is selected from a pyrroline and a thiazole, and the substitution is selected from ═O, ═S, and an electrophile. In a further example, the compound is selected from

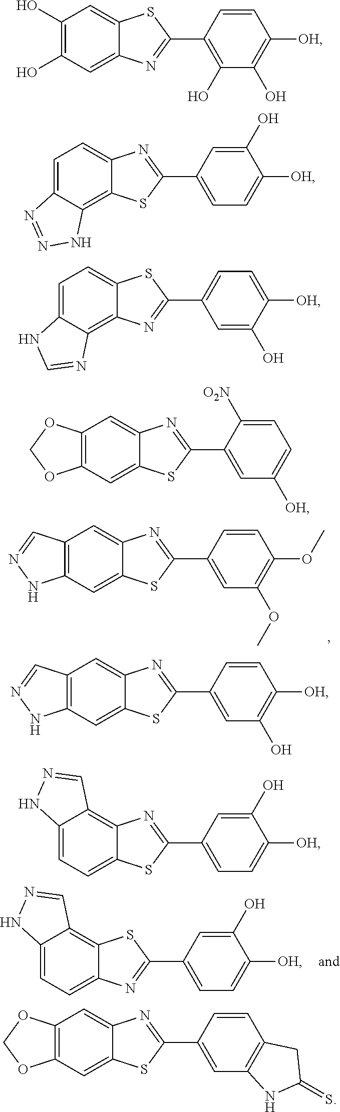

In another example, the electrophile is selected from

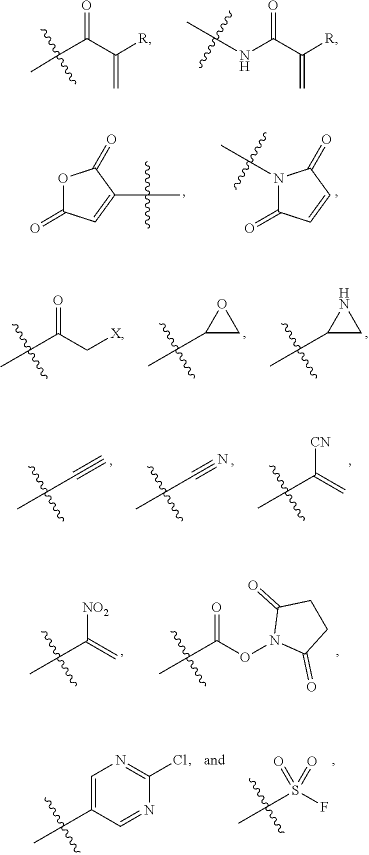

wherein R═H or any alkyl or any carbonyl, and X=any halogen, ether, amine, thiol, or thioether. In yet another example, the compound is not substituted with an electrophile.

In a further aspect, disclosed is a pharmaceutical composition, including any of the foregoing compounds of Formula la or a pharmaceutical salt thereof, and a pharmaceutically acceptable excipient.

In a further aspect, provided is a compound, including Formula Ia:

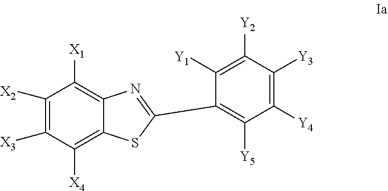

or a pharmaceutically acceptable salt thereof, wherein
X₁, X₂, X₃, and X₄ are each independently H, OH, or an electrophile, and optionally one pair selected from X₁ and X₂, X₂ and X₃, and X₃ and X₄ forms a five-membered ring including one or more heteroatom, wherein the one or more heteroatom is selected from O, N, and S, and the five-membered ring is optionally substituted with a ═C, an ═S, or an electrophile, and
Y₁, Y₂, Y₃, Y₄ and Y₅ are each independently selected from H, CH₃, OH, NO₂, and an electrophile, and optionally Y₂ and Y₃ form a five-membered ring including one or more heteroatom, wherein the one or more heteroatom is selected from O, N, and S, and the five-membered ring is optionally substituted with a ═C, ═S, or an electrophile,
with the caveats that
(a) if the pair X₂ and X₃ together form a dioxolane then optionally Y₂ and Y₃ form a five-membered ring including one or more heteroatom only if Y₂ and Y₃ form a pyrroline optionally substituted with a ═C, ═S, or an electrophile, and no more than one of Y₁, Y₂, Y₃, Y₄ and Y₅ is OH, and.
(b) if X₃ is OH and one or both of Y₃ and Y₄ are OH then Y₅ is OH

In an example, X₁ and X₂, or X₃ and X₄, form an imidazole or a triazole. In another example, X₂ and X₃ form a five-membered ring selected from a pyrrole, a dioxolane, a pyrazole, an imidazole, and a triazole. In still another example, X₂ and X₃ form a five-membered ring including a substitution, the five-membered ring is selected from a pyrroline and a tetrahydrofuran, and the substitution is selected from ═O, ═S, and an electrophile. In yet another example, X₂ and X₃ form a thiazole including a substitution, and the substitution is selected from ═O, ═S, and an electrophile. In a further example, X₂ and X₃ form a dioxolane.

In an example, Y₂ and Y₃ form a five-membered ring. In another example, Y₂ and Y₃ form a furan, a pyrrole, or a thiophene. In still another example, Y₂ and Y₃ form a five-membered ring including a substitution, wherein the five-membered ring is selected from a pyrroline and a thiazole, and the substitution is selected from ═O, ═S, and an electrophile. In yet another example, the compound is selected from

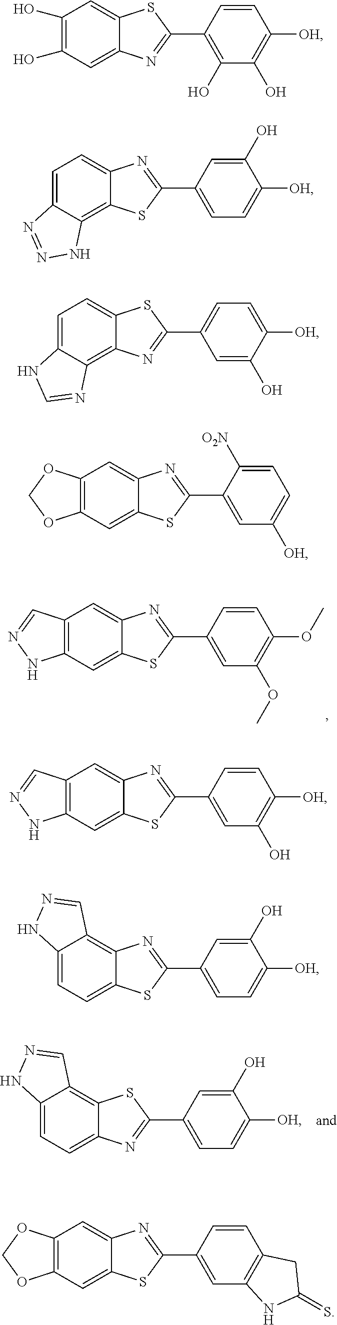

In a further example, the electrophile is selected from

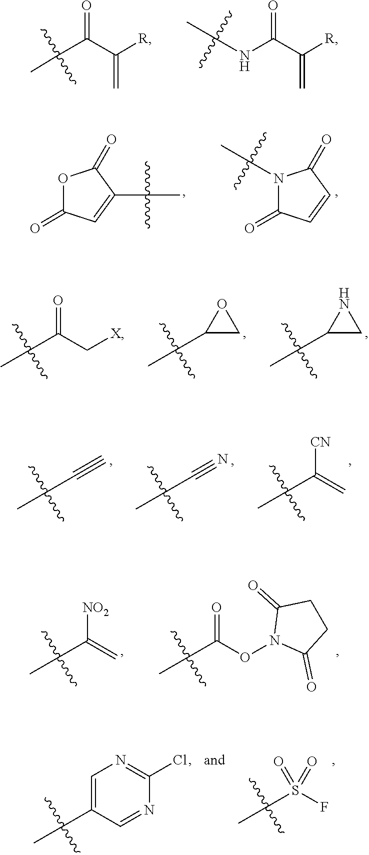

wherein R═H or any alkyl or any carbonyl, and X=any halogen, ether, amine, thiol, or thioether. In still a further example, the compound is not substituted with an electrophile.

In another aspect, disclosed is a pharmaceutical composition, including any of the foregoing compounds of Formula Ia or a pharmaceutically acceptable salt thereof and a pharmaceutically acceptable excipient.

In another aspect, disclosed is a method, including administering any of the foregoing compounds of Formula I or Formula Ia or a pharmaceutically acceptable salt thereof, or any of the foregoing pharmaceutical compositions, to a subject, wherein the subject is diagnosed with or at risk of developing cancer. In an example, the method further includes administering an epidermal growth factor receptor (EGFR) inhibitor in combination with the compound, pharmaceutically acceptable sale thereof, or pharmaceutical composition, or wherein the pharmaceutical composition further includes the EGFR inhibitor. In still another example, the EGFR inhibitor includes AZD9291. In yet another example, the cancer is selected from acute megakaryoplastic leukemia, glioblastoma, and non-small cell lung cancer.

In still another aspect, disclosed is a method, including treating an impairment in a subject, wherein the impairment includes a cognitive impairment or an affective impairment, and the treating includes administering any of the foregoing compounds of Formula I or Formula Ia or a pharmaceutically acceptable salt thereof, or any of the foregoing pharmaceutical compositions to the subject. In an example, the subject is diagnosed with Down syndrome or trisomy 21. In another example, the subject is diagnosed with or at risk for developing Alzheimer's disease.

In yet another aspect, disclosed is a method, including administering any of the foregoing compounds of Formula I or Formula la or a pharmaceutically acceptable salt thereof, or any of the foregoing pharmaceutical compositions to a subject, wherein the subject is diagnosed with or at risk for developing Alzheimer's disease.

In a further aspect, disclosed is a method, including administering any of the foregoing compounds of Formula I or Formula Ia or a pharmaceutically acceptable salt thereof, or any of the foregoing pharmaceutical compositions to a subject, wherein the subject is diagnosed with Down syndrome or trisomy 21.

### BRIEF DESCRIPTION OF THE DRAWINGS

These and other features, aspects, and advantages of the present disclosure will become better understood when the following detailed description is read with reference to the accompanying drawings, wherein:

FIG. 1 shows structural comparisons of various DYRK isoforms.

FIG. 2A shows a ribbon diagram of DYRK1A. FIG. 2B. identifies some features of the DYRK1A kinase domain. The Tyr autophosphorylation site is located ˜20 bp upstream of the SPE motif between subdomains VII and VIII (Tyr 321). FIG. 2C identifies some features of DYRK1A's substrate specificity.

FIG. 3A shows a ribbon diagram of PIM1. FIG. 3B. identifies some structural features and substrate specificity of the DYRK1A kinase domain.

FIGS. 4A-4C show effects of FC-2, FC-3, and the DYRK1A inhibitor INDY, respectively, on proliferation of the glioblastoma cell line U87MG cells in the neurosphere proliferation assay. Dot plot quantifying the diameter of neurospheres in the U87MG cell line after treatment with (A) FC-2 (B) FC-3 and (C) INDY at the indicated concentrations. Each dot represents an individual neurosphere. **p<0.005 ***p<0.0005 FIG. 5. DYRK inhibition using FC-2 and FC-3 reduces the invasive ability of U87MG cells. Quantification of the average number of cells per field that were able to cross a matrigel membrane in the invasion assay in U87MG following treatment with FC-2 and FC-3 at indicated concentrations. Averages represent two independent trials in which 15-20 fields were counted. Bars represent mean±SEM. ***p<0.0005

FIG. 5 shows effects of FC-2 and FC-3 on U87MG cells in a cell invasion assay.

FIG. 6 shows the crystal structure of DYRK1A with a compound as disclosed herein (FC-3).

FIG. 7 shows amino acid alignment of the hinge region of DYRK and PIM kinases.

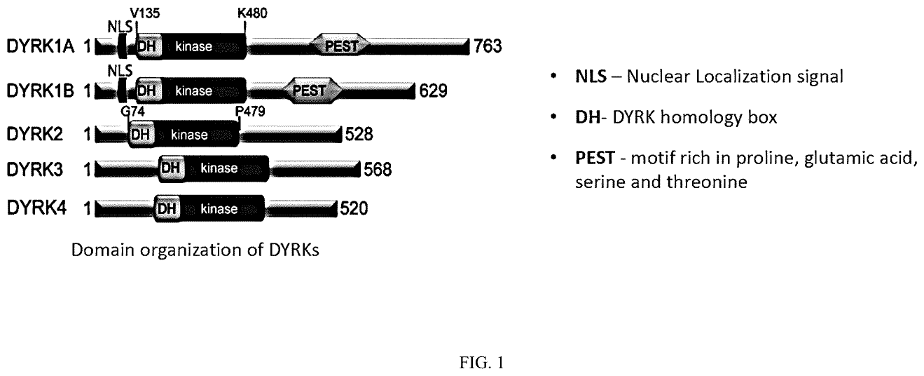

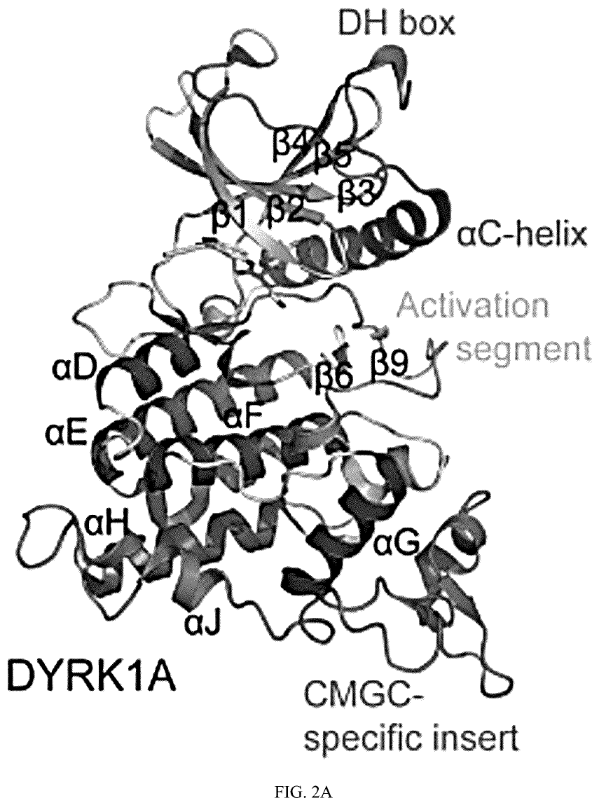

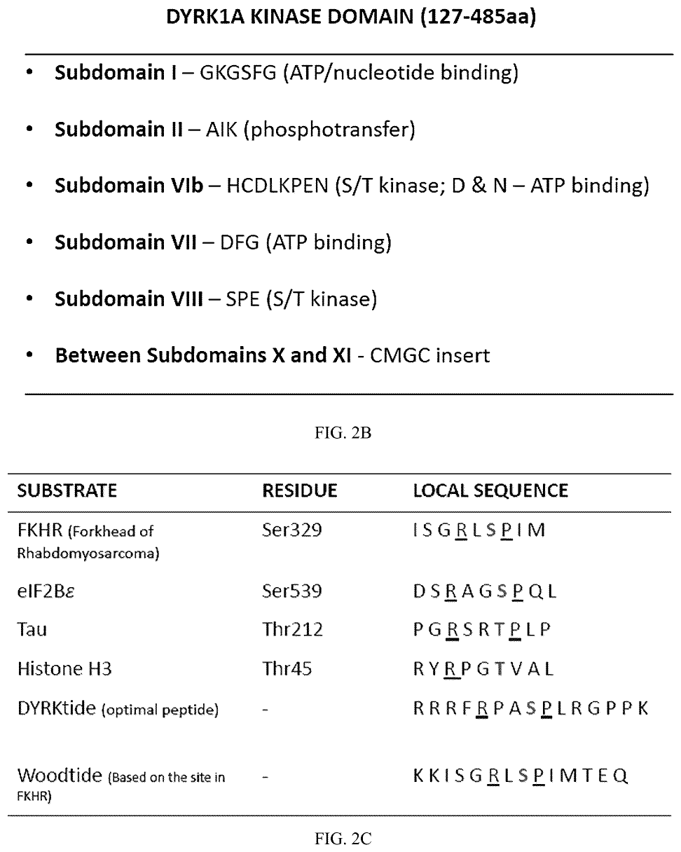

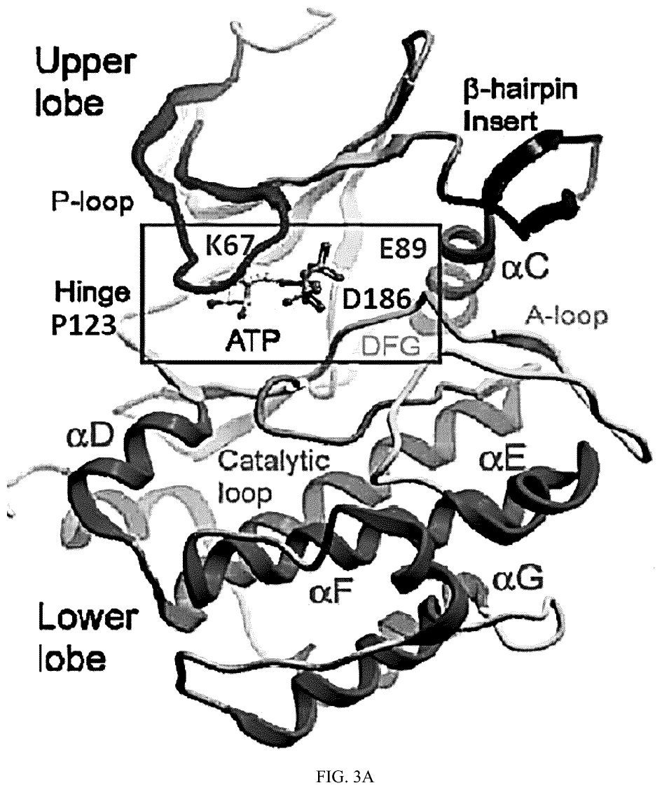

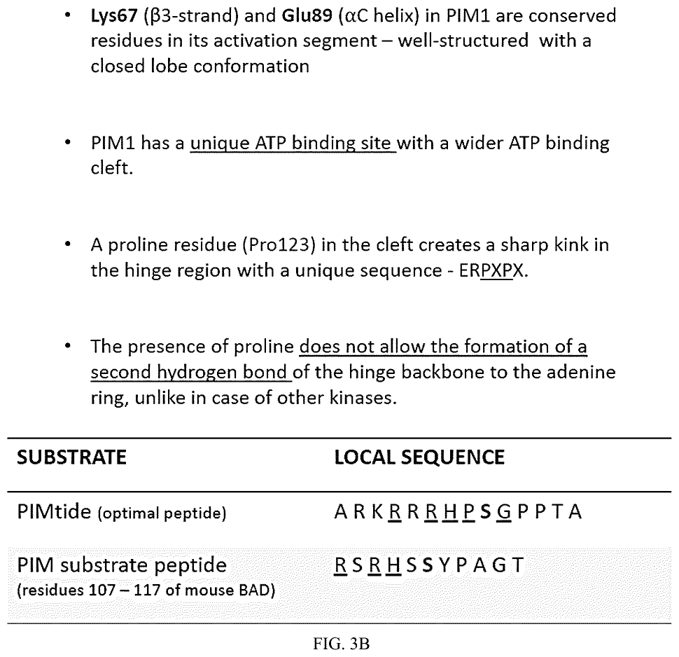

### DETAILED DESCRIPTION

This disclosure relates to compounds that may inhibit DYRK1A, compounds that may inhibit PIM1, and compounds that may inhibit DYRK1A and PIM1. In an aspect, disclosed is a compound, including a compound of Formula I:

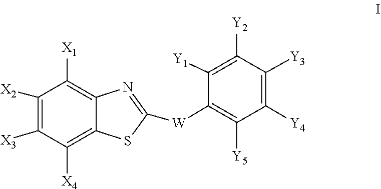

or a pharmaceutically acceptable salt thereof, wherein W is a direct bond or an optionally substituted C, and
if W is a direct bond, then X₁, X₂, X₃, and X₄ are each independently H, OH, or an electrophile, and optionally one pair selected from X₁ and X₂, X₂ and X₃, and X₃ and X₄ forms a five-membered ring including one or more heteroatom, wherein the one or more heteroatom is selected from O, N, and S, and the five-membered ring is optionally substituted with a ═C, an ═S, or an electrophile, and
Y₁, Y₂, Y₃, Y₄ and Y₅ are each independently selected from H, CH₃, OH, O—CH₃, NO₂, and an electrophile, and optionally Y₂ and Y₃ form a five-membered ring including one or more heteroatom, wherein the one or more heteroatom is selected from O, N, and S, and the five-membered ring is optionally substituted with a ═C, ═S, or an electrophile,
with the caveats that
(a) if the pair X₂ and X₃ together form a dioxolane then optionally Y₂ and Y₃ form a five-membered ring including one or more heteroatom only if Y₂ and Y₃ form a pyrroline optionally substituted with a ═C, ═S, or an electrophile, and no more than one of Y₁, Y₂, Y₃, Y₄ and Y₅ is OH,
(b) if one or more of Y₁, Y₂, Y₃, Y₄ and Y₅ is O—CH₃, then X₂ and X form a five-membered ring including one or more heteroatom, wherein the one or more heteroatom is selected from N and S, and the five-membered ring is optionally substituted with a ═C, ═S, or an electrophile
(c) if X₃ is OH and one or both of Y₃ and Y₄ are OH then Y₅ is OH; and
(d) if W is an optionally substituted C, then
   (i) at least one of X₁, X₂, X₃, X₄ is independently selected from OH, O—CH₃, and a halogen, at least one of Y₁, Y₂, Y₃, Y₄, and Y₅ is independently selected from OH, O—CH₃, and a halogen, no more than five of X₁, X₂, X₃, X₄, Y₁, Y₂, Y₃, Y₄, and Y₅ is H, and no more than one of X₁, X₂, X₃, X₄, Y₁, Y₂, Y₃, Y₄, and Y₅, is O—CH₃, or
   (ii) X₁, X₄, Y₁, Y₄, and Y₅ are hydrogen, X₂ and X₃ are OH or together form a dioxolane, and Y₂ and Y₃ are OH or together form a dioxolane.

In an example, W is —C(═O)—. In still another example, wherein at least one of X₁, X₂, X₃, X₄ is independently selected from OH, O—CH₃, and a halogen, at least one of Y₁, Y₂, Y₃, Y₄, and Y₅ is independently selected from OH, O—CH₃, and a halogen, no more than five of X₁, X₂, X₃, X₄, Y₁, Y₂, Y₃, Y₄, and Y₅ is H, and no more than one of X₁, X₂, X₃, X₄, Y₁, Y₂, Y₃, Y₄, and Y₅, is O—CH₃. In still a further example, X₁, X₄, Y₁, Y₄, and Y₅ are hydrogen, X₂ and X₃ are OH or together form a dioxolane, and Y₂ and Y₃ are OH or together form a dioxolane

In another example, the compound is selected from

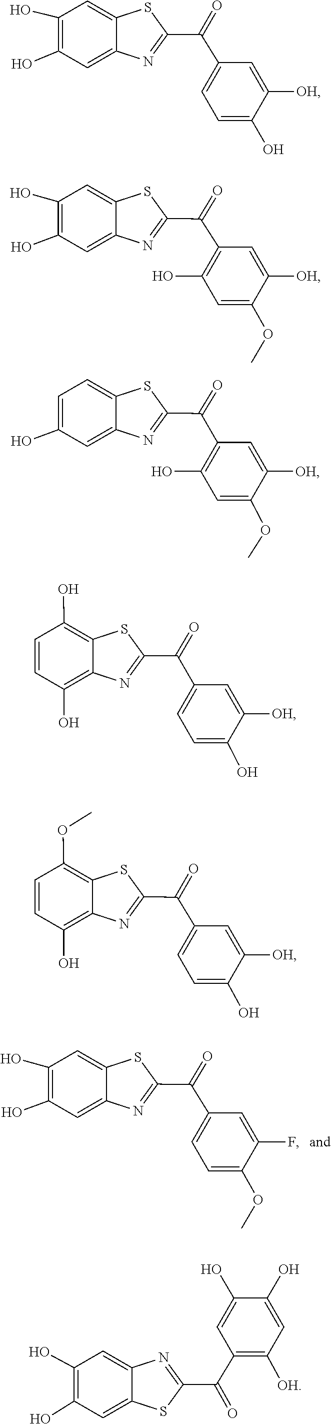

In an example, W is a direct bond. For example, the compound may include Formula Ia:

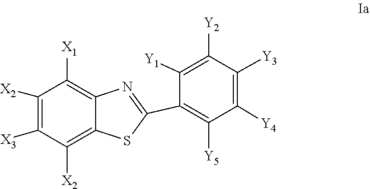

or a pharmaceutically acceptable salt thereof. In an example, X₁ and X₂, or X₃ and X₄, form an imidazole or a triazole. In still another example, X₂ and X₃ form a five-membered ring selected from a pyrrole, a dioxolane, a pyrazole, an imidazole, and a triazole. In yet another example, X₂ and X₃ form a five-membered ring including a substitution, the five-membered ring is selected from a pyrroline and a tetrahydrofuran, and the substitution is selected from ═O, ═S, and an electrophile. In a further example, X₂ and X₃ form a thiazole comprising a substitution, and the substitution is selected from ═O, ═S, and an electrophile. In still a further example, X₂ and X₃ form a dioxolane. In yet a further example, Y₂ and Y₃ form a five-membered ring. In another example, Y₂ and Y₃ form a furan, a pyrrole, or a thiophene. In another example, Y₂ and Y₃ form a five-membered ring including a substitution, wherein the five-membered ring is selected from a pyrroline and a thiazole, and the substitution is selected from ═O, ═S, and an electrophile.

In another example, the compound is selected from

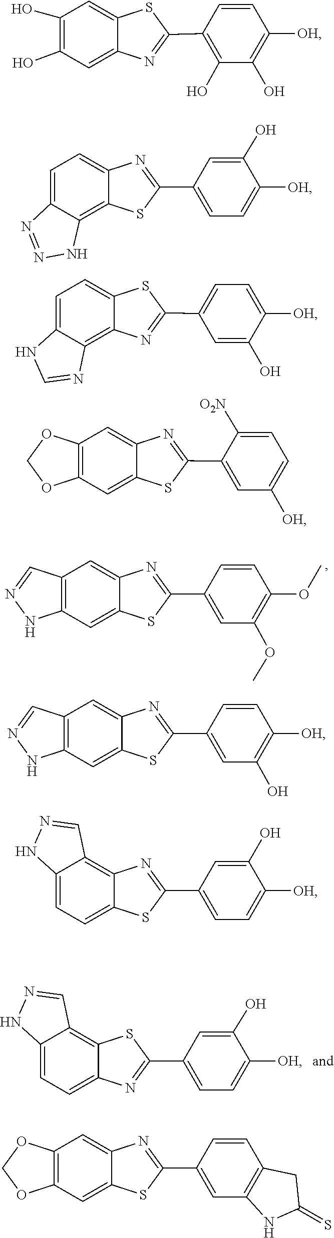

In another aspect, disclosed in a pharmaceutical composition, including any one of the foregoing compound of Formula I, or a pharmaceutically acceptable salt thereof, and a pharmaceutically acceptable excipient. In an example, W is an optionally substituted C. In another example, W comprises —C(═O)—. In still another example, wherein at least one of X₁, X₂, X₃, X₄ is independently selected from OH, O—CH₃, and a halogen, at least one of Y₁, Y₂, Y₃, Y₄, and Y₅ is independently selected from OH, O—CH₃, and a halogen, no more than five of X₁, X₂, X₃, X₄, Y₁, Y₂, Y₃, Y₄, and Y₅ is H, and no more than one of X₁, X₂, X₃, X₄, Y₁, Y₂, Y₃, Y₄, and Y₅, is O—CH₃. In still a further example, X₁, X₄, Y₁, Y₄, and Y₅ are hydrogen, X₂ and X₃ are OH or together form a dioxolane, and Y₂ and Y₃ are OH or together form a dioxolane

In another example, the compound or pharmaceutically acceptable salt thereof included in the pharmaceutical is selected from:

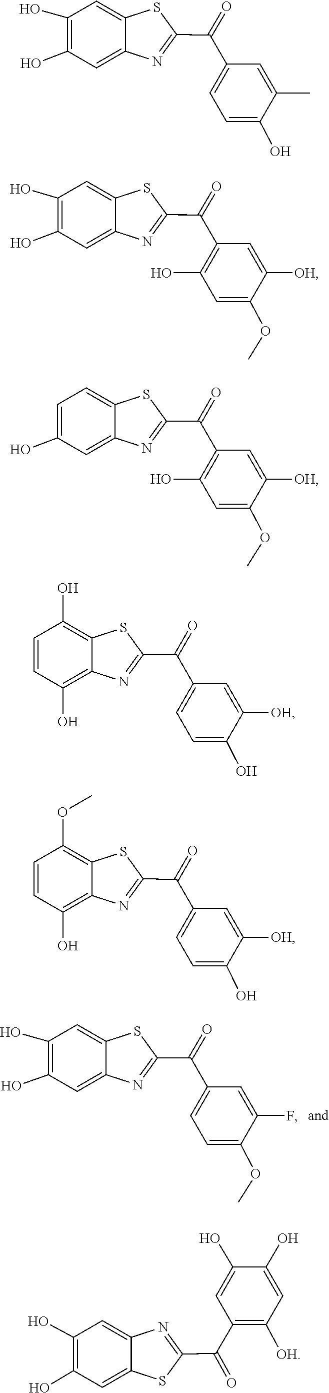

In another aspect, the compound included in the pharmaceutical composition includes any one of the foregoing compound of Formula la or pharmaceutically acceptable salt thereof. In an example, X₁ and X₂, or X₃ and X₄, form an imidazole or a triazole. In another example, X₂ and X₃ form a five-membered ring selected from a pyrrole, a dioxolane, a pyrazole, an imidazole, and a triazole. In yet another example, X₂ and X₃ form a five-membered ring comprising a substitution, the five-membered ring is selected from a pyrroline and a tetrahydrofuran, and the substitution is selected from ═O, ═S, and an electrophile. In still another example, X₂ and X₃ form a thiazole comprising a substitution, and the substitution is selected from ═O, ═S, and an electrophile. In a further example, X₂ and X₃ form a dioxolane. In yet a further example, Y₂ and Y₃ form a five-membered ring. In still a further example, Y₂ and Y₃ form a furan, a pyrrole, or a thiophene. In another example, Y₂ and Y₃ form a five-membered ring comprising a substitution, wherein the five-membered ring is selected from a pyrroline and a thiazole, and the substitution is selected from ═O, ═S, and an electrophile.

In another example, the compound or pharmaceutically acceptable salt thereof included in the pharmaceutical is selected from

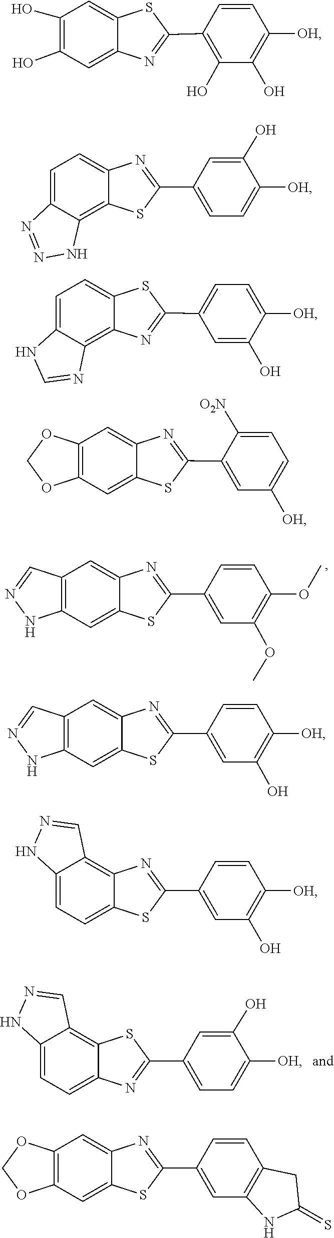

In another example, the electrophile is selected from

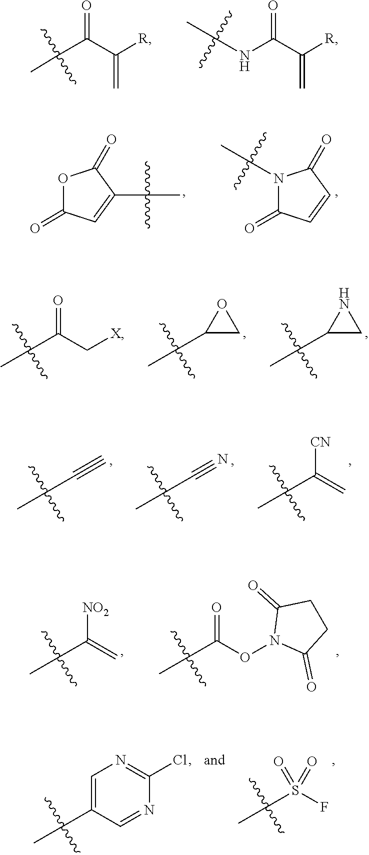

wherein R═H or any alkyl or any carbonyl, and X=any halogen, ether, amine, thiol, or thioether. In another example, the compound is not substituted with an electrophile.

In yet another aspect, provided is method including administering any one or more of the foregoing pharmaceutical compositions to a subject, wherein the subject is diagnosed with or at risk of developing cancer. An example further includes administering an epidermal growth factor receptor (EGFR) inhibitor in combination with the pharmaceutical composition, or wherein the pharmaceutical composition further comprises the EGFR inhibitor. In another example, the EGFR inhibitor includes AZD9291. In another example, the cancer is selected from acute megakaryoplastic leukemia, glioblastoma, and non-small cell lung cancer.

In still another aspect, provided is method including treating an impairment in a subject, wherein the impairment includes a cognitive impairment or an affective impairment, and the treating comprises administering any one or more of the foregoing pharmaceutical compositions to the subject. In an example, the subject is diagnosed with Down syndrome, or trisomy 21. In another example, the subject is diagnosed with or at risk for developing Alzheimer's disease.

In a further aspect, provided is method including administering any one or more of the foregoing pharmaceutical compositions to a subject, wherein the subject is diagnosed with Alzheimer's disease. In another aspect, provided is method including administering any one or more of the foregoing pharmaceutical compositions to a subject, wherein the subject is diagnosed with Down syndrome, or trisomy 21.

In an example where X₁ and X₂, X₂ and X₃, or X₃ and X₄ form a five-membered ring including one or more heteroatom, wherein the one or more heteroatom is selected from O, N, and S, and the five-membered ring is optionally substituted with a ═C, ═S, or an electrophile, the five-membered ring so formed may be selected from: a tetrahydrofuran selected from

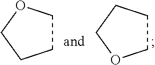

a dioxolane

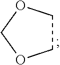

a pyrroline selected from

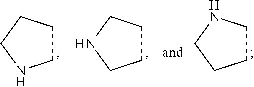

a pyrazole selected from

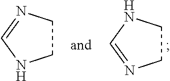

an imidazole selected from

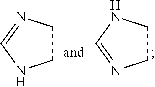

and a triazole selected from

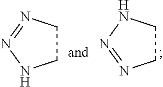

wherein the dotted line represents a carbon-carbon bond of the aromatic ring of which the five-membered ring is a substitution.

In an example where Y₂ and Y₃ may form a five-membered ring including one or more heteroatom, wherein the one or more heteroatom is selected from O, N, and S, and the five-membered ring is optionally substituted with a ═C, ═S, or an electrophile, the five-membered ring so formed may be selected from

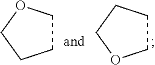

a dioxolane

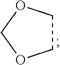

a pyrroline selected from

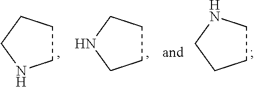

a pyrazole selected from

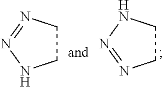

an imidazole selected from

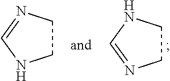

and a triazole selected from

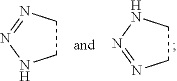

wherein the dotted line represents a carbon-carbon bond of the aromatic ring of which the five-membered ring is a substitution.

Unless otherwise specified herein, C₁ to Cₙ hydrocarbon, wherein n may be any integer from 1 to 20 or higher, includes alkyl, cycloalkyl, polycycloalkyl, alkenyl, alkynyl, aryl and combinations thereof. Examples include benzyl, phenethyl, cyclohexylmethyl, adamantyl, camphoryl and naphthylethyl. Hydrocarbyl refers to any substituent comprised of hydrogen and carbon as the only elemental constituents. Aliphatic hydrocarbons are hydrocarbons that are not aromatic; they may be saturated or unsaturated, cyclic, linear or branched. Examples of aliphatic hydrocarbons include isopropyl, 2-butenyl, 2-butynyl, cyclopentyl, norbornyl, etc. Aromatic hydrocarbons include benzene (phenyl), naphthalene (naphthyl), anthracene, etc.

Unless otherwise specified herein, alkyl (or alkylene) is intended to include linear or branched saturated hydrocarbon structures and combinations thereof. Alkyl refers to alkyl groups from 1 to 20 carbon atoms, preferably 1 to 10 carbon atoms, more preferably 1 to 6 carbon atoms. Examples of alkyl groups include methyl, ethyl, propyl, isopropyl, n-butyl, s-butyl, t-butyl and the like.

Unless otherwise specified herein, cycloalkyl is a subset of hydrocarbon and includes cyclic hydrocarbon groups of from 3 to 8 carbon atoms. Examples of cycloalkyl groups include cy-propyl, cy-butyl, cy-pentyl, norbornyl and the like.

Unless otherwise specified herein, the term “carbocycle” is intended to include ring systems in which the ring atoms are all carbon but of any oxidation state. Thus (C₃-C₁₀) carbocycle refers to both non-aromatic and aromatic systems, including such systems as cyclopropane, benzene and cyclohexene; (C₈-C₁₂) carbopolycycle refers to such systems as norbornane, decalin, indane and naphthalene. Carbocycle, if not otherwise limited, refers to monocycles, bicycles and polycycles.

Unless otherwise specified herein, heterocycle means an aliphatic or aromatic carbocycle residue in which from one to four carbons is replaced by a heteroatom selected from the group consisting of N, O, and S. The nitrogen and sulfur heteroatoms may optionally be oxidized, and the nitrogen heteroatom may optionally be quaternized. Unless otherwise specified, a heterocycle may be non-aromatic (heteroaliphatic) or aromatic (heteroaryl). Examples of heterocycles include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. Examples of heterocyclyl residues include piperazinyl, piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl (also historically called thiophenyl), benzothienyl, thiamorpholinyl, oxadiazolyl, triazolyl and tetrahydroquinolinyl.

Unless otherwise specified herein, alkoxy or alkoxyl refers to groups of from 1 to 20 carbon atoms, preferably 1 to 10 carbon atoms, more preferably 1 to 6 carbon atoms of a straight or branched configuration attached to the parent structure through an oxygen. Examples include methoxy, ethoxy, propoxy, isopropoxy and the like. Lower-alkoxy refers to groups containing one to four carbons. For the purpose of this application, alkoxy and lower alkoxy include methylenedioxy and ethylenedioxy.

Unless otherwise specified herein, oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, 196, but without the restriction of 127(a)], i.e. it refers to compounds in which the oxygen is bonded via a single bond to its adjacent atoms (forming ether bonds); it does not refer to doubly bonded oxygen, as would be found in carbonyl groups. Similarly, thiaalkyl and azaalkyl refer to alkyl residues in which one or more carbons has been replaced by sulfur or nitrogen, respectively. Examples include ethylaminoethyl and methylthiopropyl.

The term “halogen” means fluorine, chlorine, bromine or iodine atoms. In one embodiment, halogen may be a fluorine or chlorine atom.

Unless otherwise specified herein, acyl refers to formyl and to groups of 1, 2, 3, 4, 5, 6, 7 and 8 carbon atoms of a straight, branched, cyclic configuration, saturated, unsaturated and aromatic and combinations thereof, attached to the parent structure through a carbonyl functionality. Examples include acetyl, benzoyl, propionyl, isobutyryl and the like. Lower-acyl refers to groups containing one to four carbons. The double bonded oxygen, when referred to as a substituent itself is called “oxo”.

Unless otherwise specified herein, the term “optionally substituted” may be used interchangeably with “unsubstituted or substituted”. The term “substituted” may refer to the replacement of one or more hydrogen atoms in a specified group with a specified radical. For example, unless otherwise specified herein, substituted alkyl, aryl, cycloalkyl, heterocyclyl etc. refer to alkyl, aryl, cycloalkyl, or heterocyclyl wherein one or more H atoms in each residue are replaced with halogen, haloalkyl, alkyl, acyl, alkoxyalkyl, hydroxy lower alkyl, carbonyl, phenyl, heteroaryl, benzenesulfonyl, hydroxy, lower alkoxy, haloalkoxy, oxaalkyl, carboxy, alkoxycarbonyl [—C(═O)O-alkyl], alkoxycarbonylamino [HNC(═O)O-alkyl], aminocarbonyl (also known as carboxamido) [—C(═O)NH₂], alkylaminocarbonyl [—C(═O)NH-alkyl], cyano, acetoxy, nitro, amino, alkylamino, dialkylamino, (alkyl)(aryl)aminoalkyl, alkylaminoalkyl (including cycloalkylaminoalkyl), dialkylaminoalkyl, dialkylaminoalkoxy, heterocyclylalkoxy, mercapto, alkylthio, sulfoxide, sulfone, sulfonylamino, alkylsulfinyl, alkylsulfonyl, acylaminoalkyl, acylaminoalkoxy, acylamino, amidino, aryl, benzyl, heterocyclyl, heterocyclylalkyl, phenoxy, benzyloxy, heteroaryloxy, hydroxyimino, alkoxyimino, oxaalkyl, aminosulfonyl, trityl, amidino, guanidino, ureido, benzyloxyphenyl, and benzyloxy. “Oxo” may also be included among the substituents referred to in “optionally substituted”; it will be appreciated by persons of skill in the art that, because oxo is a divalent radical, there are circumstances in which it will not be appropriate as a substituent (e.g. on phenyl). In an embodiment, 1, 2, or 3 hydrogen atoms may be replaced with a specified radical. In the case of alkyl and cycloalkyl, more than three hydrogen atoms may be replaced by fluorine; indeed, all available hydrogen atoms may be replaced by fluorine.

Substituents Rⁿ are generally defined when introduced and retain that definition throughout the specification and in all independent claims. For any and all compounds shown or claimed, wherein tautomerism is possible, all possible tautomers are intended to be included.

As disclosed herein, compounds of Formula I may be inhibitors of DYRK1A. As disclosed herein, compounds of Formula I may be inhibitors of PIM1. As further disclosed herein, compounds of Formula I may have an anti-tumor profile, including an anti-proliferative effect on glioblastoma cells. Compounds may be used in the treatment of cancer, including glioblastoma or other brain or central nervous system cancers, or non-small cell lung cancer. As further disclosed herein, compounds of Formula I may be used in the treatment of AMKL. As further disclosed herein, compounds of Formula I may be used in the treatment of DS. As further disclosed herein, compounds of Formula I may be used in the treatment of AD.

A pharmaceutical composition including a compound of Formula I includes, as a non-limiting example, such compound in a lyophilized or dry form such that dissolving such dry form in solvent, including upon oral administration to a subject, such compound would bind with copper as administered therewith in solution. Formulations for administration to a subject include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of a recipient or intended purpose of the administration. A formulation may conveniently be presented in unit dosage form and may be prepared by any of the methods well known in the art of pharmacy. Methods may include a step of bringing into association a compound of Formula I or a pharmaceutically acceptable salt thereof (“active ingredient”) with a carrier which constitutes one or more accessory ingredients. In general, formulations may be prepared by uniformly and intimately bringing into association an active ingredient with liquid carriers or finely divided solid carriers or both and then, if necessary, shaping the product into the desired formulation.

Formulations of the present disclosure suitable for oral administration may be presented as discrete units such as capsules, cachets or tablets each containing a predetermined amount of an active ingredient; as a powder or granules; as a solution or a suspension in an aqueous liquid or a non-aqueous liquid; or as an oil-in-water liquid emulsion or a water-in-oil liquid emulsion. A compound of Formula I may also be presented as a bolus, electuary or paste. For oral or other administration, a compound of Formula I may be suspended in a solution, or dissolved in a solvent, such as alcohol, DMSO, water, saline, or other solvent, which may be further diluted or dissolved in another solution or solvent, and may or may contain a carrier or other excipient in some examples.

Formulations for parenteral or other administration include aqueous and non-aqueous sterile injection solutions which may contain anti-oxidants, buffers, bacteriostats and solutes which render a formulation isotonic with the blood of the intended recipient. Formulations for parenteral or other administration also may include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of a compound of Formula I to polymer and the nature of the particular polymer employed, the rate of a compound of Formula I release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

A compound of Formula I formulation may include different types of carriers depending on whether it is to be administered in solid, liquid or aerosol form, and whether it needs to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990.

The term “pharmaceutically acceptable salt” refers to salts prepared from pharmaceutically acceptable non-toxic acids or bases including inorganic acids and bases and organic acids and bases. Unless otherwise specified, reference herein to a compound of Formula I, or to any such compound in particular, includes reference to a pharmaceutically acceptable salt thereof. When the compounds of the present disclosure are basic, salts may be prepared from pharmaceutically acceptable non-toxic acids including inorganic and organic acids. Suitable pharmaceutically acceptable acid addition salts for the compounds of the present invention include acetic, adipic, alginic, ascorbic, aspartic, benzenesulfonic (besylate), benzoic, betulinic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, ursolic and the like. When the compounds contain an acidic side chain, suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

As used herein, the term “effective amount” means an amount of a compound of Formula I pharmaceutical agent that may elicit a biological or medical response of a cell, tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula I, as well as salts, solvates, and physiological functional derivatives thereof, may be administered as the raw chemical. Additionally, the active ingredient may be presented as a pharmaceutical composition.

Pharmaceutical compositions of the present invention include an effective amount of a compound of Formula I and optionally one or more additional agents dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains a compound of Formula I and optionally one or more additional active ingredient will be known to those of skill in the art in light of the present disclosure, as exemplified by Remington's Pharmaceutical Sciences, 18th Ed. Mack Printing Company, 1990. Moreover, for animal (e.g., human) administration, it will be understood that preparations should meet sterility, pyrogenicity, general safety and purity standards as required by FDA Office of Biological Standards.

In some examples, a compound of Formula I may include a substitution to permit covalent attachment of the compound to its target, such as DYRK1A or PIM1. After protein target recognition and binding, such covalent inhibitors (CKI) may irreversibly, or reversibly, covalently attach the compound to the target enzyme. For example, an electrophilic moiety, or electrophile, may be included as a substituent in the compound of Formula I, such that recognition of its target enzyme (DYRK1A or PIM1) leads to reaction of the electrophile with a nucleophile of the enzyme, such as a cysteine thiol or other amino acid. An electrophile may be included as a substitution at any one of X₁, X₂, X₃, X₄, Y₁, Y₂, Y₃, Y₄, and Y₅. In another example, the electrophile is included as a substituent at Y₁. In another example, the electrophile is included as a substituent at Y₂. In another example, the electrophile is included as a substituent at Y₃. In another example, the electrophile is included as a substituent at Y₄. In another example, the electrophile is included as a substituent at Y₅. In another example, the electrophile is included as a substituent at X₁. In another example, the electrophile is included as a substituent at X₂. In another example, the electrophile is included as a substituent at X₃. In another example, the electrophile is included as a substituent at X₄.

Some examples of reactive electrophiles that may be included as a substituent include any of the following:

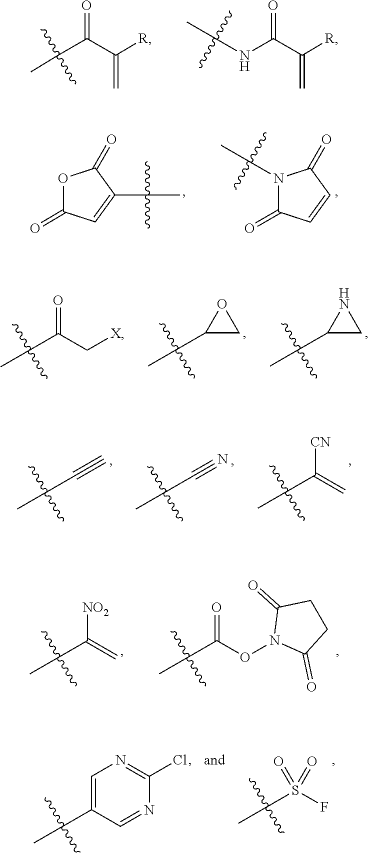

wherein R═H or any alkyl or any carbonyl, and X=any halogen, ether, amine, thiol, or thioether. Any such electrophile may be included as a substituent on any compound of Formula I, all such combinations of which being explicitly included in this disclosure.

In an example, disclosed is a compound or pharmaceutical composition for use as a medicament, wherein the compound includes any one or more of the foregoing compounds of Formula I or Formula Ia, or pharmaceutically acceptable salt or salts thereof, or the pharmaceutical composition includes any such compound or compounds or pharmaceutically acceptable salt thereof. Each and every example of compounds of Formula I or Formula Ia disclosed in herein, without exception or limitation, is expressly and explicitly included as a compound for use as a medicament as disclosed in this paragraph.

In an example, disclosed is a compound or pharmaceutical composition for use in treating cancer in a subject, wherein the compound includes any one or more of the foregoing compounds of Formula I or Formula Ia, or pharmaceutically acceptable salt or salts thereof, or the pharmaceutical composition includes any such compound or compounds or pharmaceutically acceptable salt thereof. In an example, the subject is diagnosed with or at risk of developing cancer. In another example, the use further includes administering an epidermal growth factor receptor (EGFR) inhibitor in combination with the compound, pharmaceutically acceptable sale thereof, or pharmaceutical composition, or the pharmaceutical composition further includes the EGFR inhibitor. In another example, the EGFR inhibitor includes AZD9291. In yet another example, the cancer is selected from acute megakaryoplastic leukemia, glioblastoma, and non-small cell lung cancer. Each and every example of compounds of Formula I or Formula Ia disclosed in herein, without exception or limitation, is expressly and explicitly included as a compound for use in treating cancer in a subject as disclosed in this paragraph.

In another example, disclosed is a compound or pharmaceutical composition for use in treating an impairment in a subject, wherein the impairment includes a cognitive impairment or an affective impairment in a subject, wherein the compound includes any one or more of the foregoing compounds of Formula I or Formula Ia, or pharmaceutically acceptable salt or salts thereof, or the pharmaceutical composition includes any such compound or compounds or pharmaceutically acceptable salt thereof. In still another example, the subject is diagnosed with Down syndrome or trisomy 21. In yet another example, the subject is diagnosed with or at risk for developing Alzheimer's disease.

In an example, disclosed is a compound or pharmaceutical composition for use in treating or Alzheimer's disease in a subject, wherein the compound includes any one or more of the foregoing compounds of Formula I or Formula Ia, or pharmaceutically acceptable salt or salts thereof, or the pharmaceutical composition includes any such compound or compounds or pharmaceutically acceptable salt thereof. In in an example, the subject is diagnosed with or at risk for developing Alzheimer's disease.

In an example, disclosed is a compound or pharmaceutical composition for use in treating or Down syndrome or trisomy 21 in a subject, wherein the compound includes any one or more of the foregoing compounds of Formula I or Formula Ia, or pharmaceutically acceptable salt or salts thereof, or the pharmaceutical composition includes any such compound or compounds or pharmaceutically acceptable salt thereof. In in an example, the subject is diagnosed with or at risk for developing Down syndrome or trisomy 21.

### EXAMPLES

The following examples are intended to illustrate particular embodiments of the present disclosure, but are by no means intended to limit the scope thereof.

### Preparation of compounds of Formula I

### General

Chemicals were obtained from commercial suppliers and used without further purification unless otherwise indicated. All ¹H NMR spectra were recorded on a Bruker Avance 300, 400 or 600 MHz. The ¹³C NMR spectra were recorded at 100 or 125 MHz. Chemical shifts are relative to the deuterated solvent peak and are in ppm. The coupling constants (J) are measured in Hz. The 1H signals are described as s (singlet), d (doublet), t (triplet), q (quartet), m (multiplet), or br s (broad singlet). Low-resolution mass spectrometry was carried out at the LTQ XL™ Linear Ion Trap mass spectrometry. HPLC was performed using a Waters system combining a 1525 binary PUMP. The analytical column was a Phenomenex Kinetex 2.6 μm EVO C18 100 Å 150×4.6 mm. Chromatography was performed at ambient temperature with a flow rate of 1 mL/min with a linear gradient from Water (0.05% TFA): AcCN (0.05% TFA)[95:5] to Water (0.05% TFA): AcCN (0.05% TFA) [5:95] in 15 min, monitored/detected UV at 254 nM by Photodiode Array (PDA) Detector.

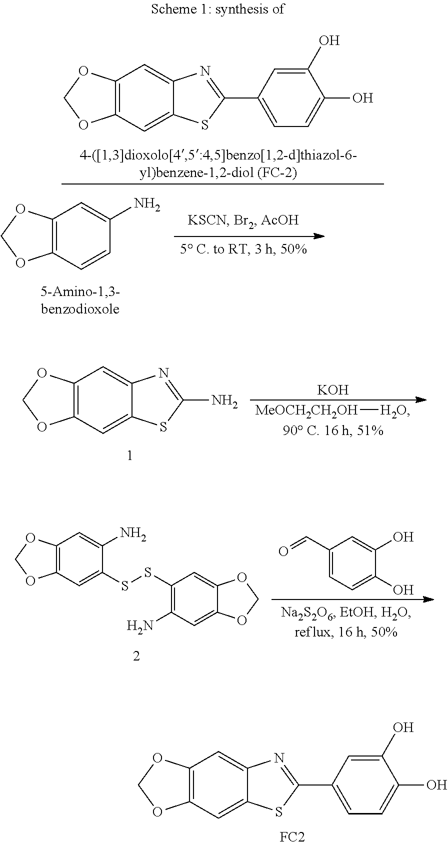

### [1,3]Dioxolo[4′,5′:4,5]benzo[1,2-d]thiazol-6-amine (1)

To a stirred solution of 5-Amino-1,3-benzodioxole (3 g, 1 eq), KSCN (8.5 g, 4 eq) in acetic acid (22 mL) at 0° C., bromine (3.5 g, 1 eq) in acetic acid (22 mL) was added dropwise at 0 to 5° C. over a period of 30 min. The reaction was allowed to warm to room temperature and further stirred for 2 h. The reaction progress was monitored by TLC analysis until complete consumption of starting material was indicated. The reaction mixture was diluted with water (100 mL) and saponified with aqueous ammonia solution (50 mL), and stirred for 30 min. The solid obtained was filtered and washed with water (1×20 mL) to obtain the crude solid compound. The crude compound was purified by column chromatography (230-400 mesh silica gel, eluent 2% methanol in DCM) to afford [1,3]dioxolo[4′,5′:4,5]benzo[1,2-d]thiazol-6-amine (1) as a pale green solid; yield 2.1 g (50%); ¹H NMR (DMSO-d6, 300 MHz) δ 7.251 (s, 1H), 7.192 (s, 2H), 6.924 (s, 1H), 5.958 (s, 2H).

### 6,6′-Disulfanediylbis(benzo[d][1,3]dioxol-5-amine) (2)

A solution of KOH (9 g) in water (9 mL) was stirred for 10 minutes at room temperature, followed by slow addition of compound 1 (1.5 g, 1 eq) and 2-methoxyethanol (9 mL) at room temperature. The reaction was heated to 90° C. and stirred for 16 h. The reaction progress was monitored by TLC analysis until complete consumption of starting material was indicated. The reaction mixture was cooled to 0° C., neutralized with acetic acid (15 mL) and stirred for 30 minutes. The mixture was extracted with ethyl acetate (2×150 mL) and washed with water (1×25 mL). The organic layer was dried over anhydrous Na₂SO₄, filtered and evaporated under reduced pressure to obtain crude compound. The crude compound was purified by column chromatography (230-400 mesh silica gel) with 20% ethyl acetate in hexane as eluent to obtain 6,6′-disulfanediylbis(benzo[d][1,3]dioxol-5-amine) (2) as a yellow solid; yield 700 mg (51%); ¹H NMR (CDCl₃, 300 MHz) δ 6.692 (s, 2H), 6.286 (s, 2H), 5.871 (s, 4H), 4.224 (br s, 4H).

### 4-([1,3]Dioxolo[4′,5′:4,5]benzo[1,2-d]thiazol-6-yl)benzene-1,2-diol (FC-2)

To a solution of compound 2 (336 mg, 1 eq) in ethanol (15 mL) was added sodium dithionate (700 mg, 4 eq) in water (5 mL) at room temperature followed by 3,4-dihydroxybenzaldehyde (280 mg, 2 eq) and the mixture was heated to reflux for 16 h. The reaction progress was monitored by TLC analysis until the starting material was consumed. The reaction mixture was cooled to room temperature and diluted with ethyl acetate (150 mL) and washed with water (1×50 mL). The organic layer was dried over anhydrous Na₂SO₄, filtered and evaporated under reduced pressure to afford crude compound. The crude compound was purified by column chromatography (230-400 mesh silica gel) with 5% methanol in DCM as eluent followed by prep HPLC to obtain 4-([1,3]dioxolo[4′,5′:4,5]benzo[1,2-d]thiazol-6-yl)benzene-1,2-diol (FC-2) as a yellow solid; yield 280 mg (50%); ¹H NMR (DMSO-d6, 400 MHz) δ 9.435 (Br s, 2H), 7.598 (s, 1H), 7.488 (s, 1H), 7.442 (d, 1H, J=2.16 Hz), 7.296 (dd, 1H, J=2.16 & 8.2 Hz), 6.864 (d, 1H, J=8.2 Hz), 6.128 (s, 2H); ¹³C-NMR (DMSO-d6, 100 MHz) δ 100.864, 101.759, 101.908, 113.499, 116.080, 118.698, 124.669, 127.001, 145.745, 146.066, 147.566, 148.387, 148.652, 165.903; HPLC purity, 96.2% (254 nm).

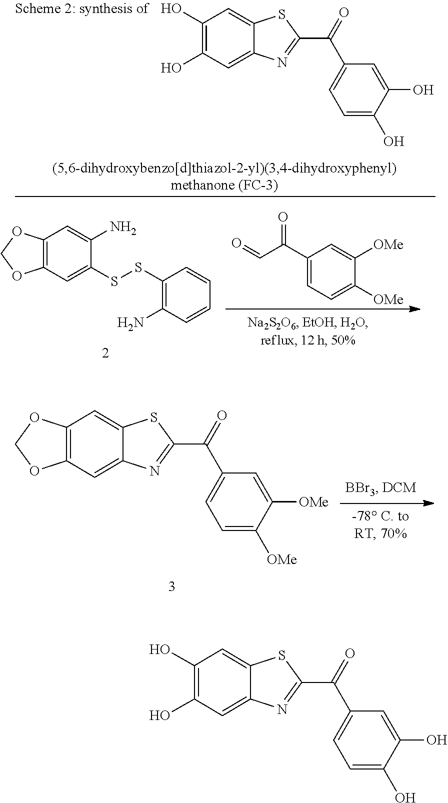 [SMILES: O=C(c1ccc(O)c(O)c1)c1nc2cc(O)c(O)cc2s1 ; Nc1ccccc1SSc1cc2c(cc1N)OCO2 ; COc1ccc(C(=O)C=O)cc1OC ; COc1ccc(C(=O)c2nc3cc4c(cc3s2)OCO4)cc1OC ; O=C(c1ccc(O)c(O)c1)c1nc2cc(O)c(O)cc2s1]

### [1,3]dioxolo[4′,5′:4,5]benzo[1,2-d]thiazol-6-yl(3,4-dimethoxyphenyl)methanone (3)

To a solution of compound 2 (224 mg, 1 eq) in ethanol (15 mL) was added sodium dithionate (466 mg, 4 eq) in water (5 mL) at room temperature followed by 2-(3,4-dimethoxyphenyl)-2-oxoacetaldehyde (260 mg, 2 eq) and the reaction was heated to reflux for 12 h. The reaction progress was monitored by TLC analysis until complete consumption of starting material was observed. The reaction mixture was cooled to room temperature and diluted with ethyl acetate (150 mL) and washed with water (1×50 mL). The organic layer was dried over anhydrous Na₂SO₄, filtered and evaporated under reduced pressure to afford crude compound. The crude compound was purified by column chromatography (230-400 mesh silica gel) and eluted with 25% ethyl acetate in hexane to afford [1,3]dioxolo[4′,5′:4,5]benzo[1,2-d]thiazol-6-yl(3,4-dimethoxyphenyl)methanone (3); yield 230 mg (50%); ¹H NMR (CDCl₃, 300 MHz) δ 8.500 (dd, 1H, J=2.1 & 8.7 Hz), 8.029 (d, 1H, J=2.1 Hz), 7.556 (s, 1H), 7.337 (s, 1H), 7.009 (d, 1H, J=8.4 Hz), 6.123 (s, 2H), 3.998 (s, 3H), 3.993 (s, 3H).

### (5,6-Dihydroxybenzo[d]thiazol-2-yl)(3,4-dihydroxyphenyl)methanone (FC-3)

A stirred solution of compound 3 (180 mg, 1 eq) in DCM (10 mL) at room temperature was cooled to −78° C. in dry-ice-acetone bath. To this cooled solution was added BBr₃ (1M in DCM) (4.5 mL, 8 eq) at −78° C. and the reaction was stirred for 30 min. The reaction mixture was allowed to warm to room temperature and stirred for 16 h. The reaction progress monitored by TLC analysis indicated complete consumption of starting material. The reaction mixture was quenched with methanol at 0° C., stirred for 30 mins, and evaporated under reduced pressure to obtain crude compound. The crude compound was purified by prep HPLC to afford 5,6-dihydroxybenzo[d]thiazol-2-yl)(3,4-dihydroxyphenyl)methanone (FC-3) as a brown solid; yield 111 mg (70%); ¹H NMR (DMSO-d6, 400 MHz) δ 8.074 (d, 1H, J=8.4 Hz), 7.94 (d, 1H, J=1.7 Hz), 7.511 (s, 1H), 7.445 (s, 1H), 6.932 (d, 1H, J=8.4 Hz); ¹³C-NMR (DMSO-d6, 100 MHz) δ 105.969, 108.972, 115.329, 117.662, 124.825, 126.285, 128.526, 145.165, 147.238, 147.599, 148.508, 151.883, 164.299, 181.947; HPLC purity, 94.7% (254 nm).

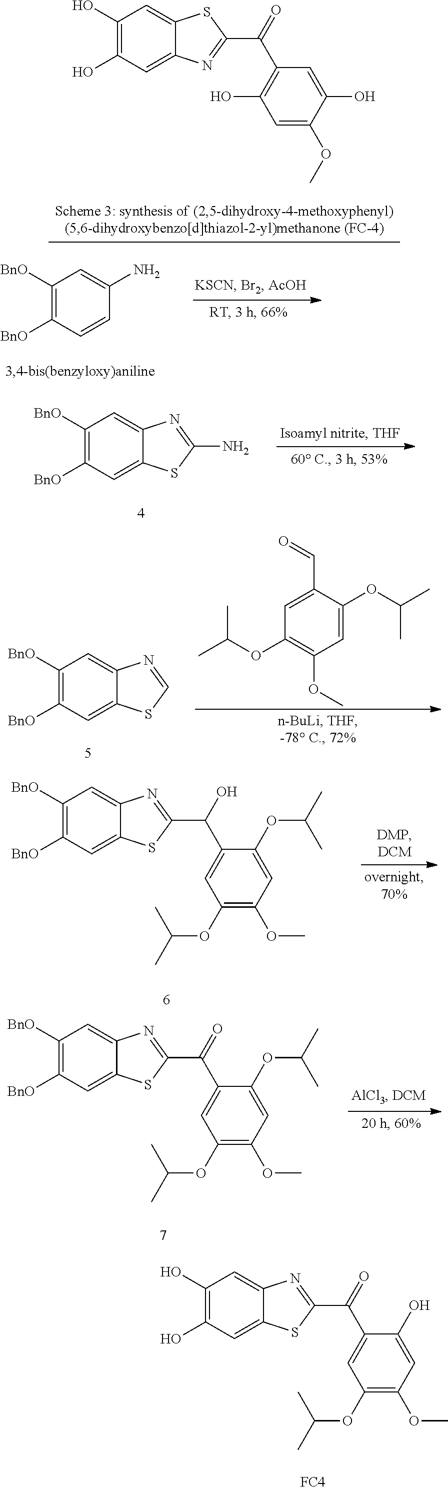

### 5,6-Bis(benzyloxy)benzo[d]thiazol-2-amine (4)

To a stirred solution of 3,4-bis(benzyloxy)aniline (6.0 g, 1 eq), KSCN (7.63 g, 4 eq) in acetic acid (47 mL) at 0° C. was added Br₂ (3.14 g, 1 eq) in acetic acid (47 mL) over a period of 30 min. After the addition of bromine, the reaction was allowed to warm to room temperature and stirred for 3 h. The reaction progress was monitored by TLC until analysis indicated complete consumption of starting material. The reaction mixture was diluted with water (100 mL), saponified with aqueous ammonia solution (50 mL), extracted with ethyl acetate (2×100 mL) and washed with brine solution (1×50 mL). The organic layer was dried over anhydrous Na₂SO₄, filtered and evaporated under reduced pressure to obtain crude compound. The crude compound was purified by column chromatography (230-400 mesh silica gel) and eluted with 40% ethyl acetate in hexane to provide 5,6-bis(benzyloxy)benzo[d]thiazol-2-amine (4) as an off white solid; yield 4.7 g (66%); ¹H NMR (CDCl₃, 300 MHz) δ 7.489-7.416 (m, 4H), 7.395-7.278 (m, 6H), 7.132 (s, 1H), 7.119 (s, 1H), 6.50-5.50 (br, 2H), 5.166 (s, 2H), 5.127 (s, 2H).

### 5,6-bis(benzyloxy)benzo[d]thiazole (5)

To a stirred solution of compound 4 (4 g, 1 eq), in THF (40 mL) was added isoamyl nitrite (3.2 g, 2.5 eq) at room temperature. The reaction mixture was heated to 60° C. and stirred for 3 h. The reaction progress was monitored by TLC until analysis indicated complete consumption of starting material. The reaction mixture was diluted with ethyl acetate (100 mL), washed with water (1×25 mL) and brine solution (1×20 mL). The organic layer was dried over anhydrous Na₂SO₄, filtered and evaporated under reduced pressure to obtain crude compound. The crude compound was purified by column chromatography (230-400 mesh silica gel) with 20% ethyl acetate in hexane to get 5,6-bis(benzyloxy)benzo[d]thiazole (5) as off white solid; yield 2.05 g (53%); ¹H NMR (CDCl₃, 300 MHz) δ 8.903 (s, 1H), 7.658 (s, 1H), 7.513-7.462 (m, 4H), 7.407-7.311 (m, 7H), 5.262 (s, 2H), 5.236 (s, 2H).

### (5,6-bis(benzyloxy)benzo[d]thiazol-2-yl)(2,5-diisopropoxy-4-methoxyphenyl)methanol (6)

A solution of compound 5 (400 mg, 1 eq) in THF (15 mL) was cooled to −78° C. and n-BuLi (2M in hexane) (0.9 mL, 1.5 eq) was added slowly dropwise at −78° C. and the reaction mixture was further stirred for 30 min. To the above cooled solution was added 2,5-diisopropoxy-4-methoxybenzaldehyde (362.7 mg, 1.2 eq) in THF (4 mL) at −78° C. The reaction was allowed to warm to room temperature and further stirred for 5 h. The reaction progress was monitored by TLC until analysis indicated complete consumption of starting material. The reaction mixture was quenched with saturated NH₄Cl solution (5 mL), extracted with ethyl acetate (3×15 mL) and washed with brine solution (1×10 mL). The organic layer dried over anhydrous Na₂SO₄, filtered and evaporated under reduced pressure to obtain crude compound. The crude compound was purified by column chromatography (230-400 mesh silica gel) with 10% ethyl acetate in hexane to obtain (5,6-bis(benzyloxy)benzo[d]thiazol-2-yl)(2,5-diisopropoxy-4-methoxyphenyl)methanol (6) as a pale yellow solid; yield 500 mg (72%). ¹H NMR (CDCl₃, 300 MHz) δ 7.519 (s, 1H), 7.486-7.442 (m, 4H), 7.390-7.304 (m, 7H), 6.961 (s, 1H), 6.509 (s, 1H), 6.132 (d, 1H, J=5.4 Hz), 4.550-4.470 (m, 1H), 4.413-4.332 (m, 1H), 4.067 (d, 1H, J=6.0 Hz), 3.831 (s, 3H), 1.342-1.259 (m, 9H), 1.166 (d, 3H, J=6.0 Hz).

### (5,6-Bis(benzyloxy)benzo[d]thiazol-2-yl)(2,5-diisopropoxy-4-methoxyphenyl)methanone (7)

To a stirred solution of compound 6 (500 mg, 1 eq) in DCM (10 mL) at 0° C. was added Dess-Martin Periodinane (DMP, 881.8 mg, 2 eq) with stirring for 30 min. The reaction mixture was allowed to warm to room temperature and stirred overnight. The reaction progress was monitored by TLC until analysis indicated complete consumption of starting material. The reaction mixture was quenched with saturated NaHCO₃ solution (10 mL), extracted with DCM (2×25 mL) and washed with brine solution (1×20 mL). The organic layer was dried over anhydrous Na₂SO₄, filtered and evaporated under reduced pressure to obtain crude compound. The crude compound was purified by column chromatography (230-400 mesh silica gel) and eluted with 10% ethyl acetate in hexane to yield (5,6-bis(benzyloxy)benzo[d]thiazol-2-yl)(2,5-diisopropoxy-4-methoxyphenyl)methanone (7) as pale yellow solid; yield 350 mg (70%); ¹H NMR (CDCl₃, 300 MHz) δ 7.586 (s, 1H), 7.503-7.440 (m, 4H), 7.415-7.315 (m, 8H), 6.529 (s, 1H), 5.273 (s, 2H), 5.241 (s, 2H), 4.473-4.371 (m, 2H), 3.904 (s, 3H), 1.356 (d, 6H, J=6.3 Hz), 1.097 (d, 6H, J=6.0 Hz).

### (2,5-Dihydroxy-4-methoxyphenyl)(5,6-dihydroxybenzo[d]thiazol-2-yl)methanone (FC-4)

To a stirred solution of compound 7 (200 mg, 1 eq) in DCM (10 mL) at 0° C. was slowly added AlCl₃ (278 mg, 5 eq) and the resulting suspension was stirred for 30 min. The reaction mixture was allowed to warm to room temperature and further stirred for 20 h. The reaction progress was monitored by TLC until analysis indicated complete consumption of starting material. The reaction mixture was quenched with conc. HCl (1 mL) and methanol (3 mL) at room temperature and stirred for 30 min at which time the reaction was evaporated under reduced pressure to obtain crude compound. The crude compound was filtered through a short plug of silica gel using MeOH-DCM (10:90) and finally purified by prep HPLC to obtain (2,5-dihydroxy-4-methoxyphenyl)(5,6-dihydroxybenzo[d]thiazol-2-yl)methanone (FC-4) as a brown solid; yield 66 mg (60%); ¹H NMR (DMSO-d6, 600 MHz) δ 8.646 (s, 1H), 7.533 (s, 1H), 7.471 (s, 1H), 6.603 (s, 1H), 3.905 (s, 3H); ¹³C-NMR (DMSO-d6, 125 MHz) δ 183.825, 163.936, 160.497, 157.079, 148.936, 147.518, 139.593, 128.724, 116.434, 109.951, 108.701, 105.958, 100.342, 56.078; HPLC purity 95.85% (254 nm).

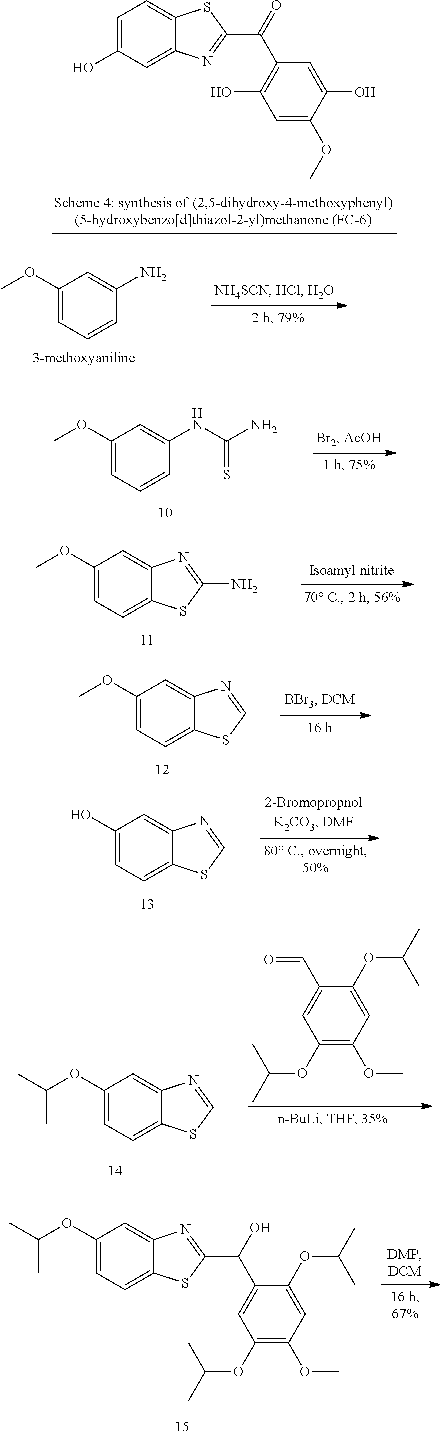

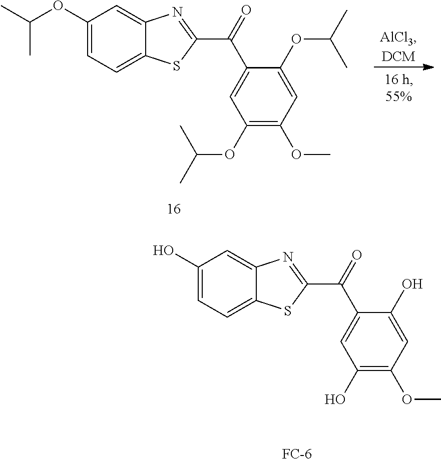 [SMILES: COc1cc(OC(C)C)c(C(=O)c2nc3cc(OC(C)C)ccc3s2)cc1OC(C)C ; COc1cc(O)c(C(=O)c2nc3cc(O)ccc3s2)cc1O ; "FC-6"]

### 1-(3-Methoxyphenyl)thiourea (10)

To a stirred solution of 3-methoxyaniline (15 g, 1 eq) in water (30 mL) and conc. HCl (15 mL) was added slowly portionwise NH₄SCN (11.12 g, 1.1 eq) at room temperature over a period of 10 min. The resulting reaction mixture was heated to 100° C. for 2 h after which time all raw material was consumed. The reaction mixture diluted with water (100 mL) and stirred for 1 h at room temperature. The solid obtained was filtered and washed with water (50 mL) to obtain crude compound. The crude compound was triturated with MTBE (2×50 mL) to obtain pure 1-(3-methoxyphenyl)thiourea (10) as white solid; yield 17.6 g (79%); ¹H NMR (DMSO-d6, 300 MHz) δ 9.661 (br s, 1H), 7.445 (br, 2H), 7.217 (t, 1H, J=8.1 Hz), 7.101 (s, 1H, J=2.1 Hz), 6.907 (dd, 1H, J=1.2 & 8.4 Hz), 6.686 (dd, 1H, J=2.4 & 8.1 Hz).

### 5-Methoxybenzo[d]thiazol-2-amine (11)

To a stirred solution of compound 10 (10 g, 1 eq) in DCM (100 mL) at 0° C. was added slowly dropwise Br₂ (9.65 g, 1.1 eq) over a period of 30 min. The resulting reaction mixture was allowed to warm to room temperature and stirred for 1 h. The reaction progress was monitored by TLC until analysis indicated complete consumption of starting material. The reaction mixture was diluted with DCM (100 mL), washed with saturated NaHCO₃ solution (2×50 mL), and brine solution (1×50 mL). The organic layer was dried over anhydrous Na₂SO₄, filtered and evaporated under reduced pressure to obtain 5-methoxybenzo[d]thiazol-2-amine (11) as a white solid; yield 7.4 g (75%); ¹H NMR (CDCl₃, 300 MHz) δ 7.435 (d, 1H, J=8.4 Hz), 7.116 (s, 1H, J=2.4 Hz), 6.766 (dd, 1H, J=2.4 & 8.7 Hz,), 5.190 (br s, 2H), 3.837 (s, 3H).

### 5-Methoxybenzo[d]thiazole (12)

To a stirred solution of 5-methoxybenzo[d]thiazol-2-amine (6 g, 1 eq) in THF (100 mL) was added isoamyl nitrite (9.7 g, 2.5 eq) at room temperature. The reaction mixture was heated to 70° C. for 2 h. The reaction progress was monitored by TLC until analysis indicated complete consumption of starting material. The reaction mixture was diluted with ethyl acetate (100 mL) and washed with brine solution (1×50 mL). The organic layer was dried over anhydrous Na₂SO₄, filtered and evaporated under reduced pressure to obtain crude compound. The crude compound was purified by column chromatography (230-400 mesh silica gel) with 1% methanol in DCM to get 5-methoxybenzo[d]thiazole (12) as an off white solid; yield 3.1 g (56%); ¹H NMR (CDCl₃, 300 MHz) δ 8.980 (s, 1H), 7.802 (d, 1H, J=8.7 Hz), 7.616 (d, 1H, J=2.1 Hz), 7.100 (dd, 1H, J=2.4 & 8.7 Hz), 3.908 (s, 3H).

### Benzo[d]thiazol-5-ol (13)

To a stirred solution of 5-methoxybenzo[d]thiazole (3 g, 1 eq) in DCM (70 mL) at −78° C. was slowly added BBr₃ (1 M in DCM) (37 mL, 2 eq) with stirring for 30 min. The reaction mixture was allowed to warm to room temperature and stirred for 16 h. The reaction progress was monitored by TLC until analysis indicated complete consumption of starting material. The reaction mixture was cooled to −78° C., quenched with methanol (15 mL) and further stirred for 30 min at room temperature. The reaction mixture was evaporated under reduced pressure to obtain crude compound. The crude product was dissolved in ethyl acetate (100 mL), washed with saturated NaHCO₃ solution (1×20 mL) and brine solution (1×20 mL). The organic layer was dried over anhydrous Na₂SO₄, filtered and evaporated under reduced pressure to obtain crude compound. The crude compound was triturated with n-pentane to produce benzo[d]thiazol-5-ol (13) (2.6 g) as a pale brown solid. The crude product was directly used for the next step without further purification.

### 5-Isopropoxybenzo[d]thiazole (14)

To a stirred suspension of benzo[d]thiazol-5-ol (2.6 g, 1 eq), K₂CO₃ (7.1 g, 3 eq) and 18 crown-6 (0.1 g) in DMF (25 mL) was added isopropyl bromide (3.17 g, 1.5 eq) at room temperature. The reaction mixture was heated to 80° C. and stirred overnight. The reaction progress was monitored by TLC until analysis indicated complete consumption of starting material. The reaction mixture was diluted with MTBE (100 mL) and washed with water (3×25 mL) and brine solution (1×25 mL). The organic layer was dried over anhydrous Na2SO+, filtered and evaporated under reduced pressure to obtain crude compound. The crude compound was purified by column chromatography (230-400 mesh silica gel) with 1% methanol in DCM to provide 5-isopropoxybenzo[d]thiazole (14) as pale brown liquid; yield 1.75 g (50% in 2 steps); ¹H NMR (CDCl₃, 300 MHz) δ 8.960 (s, 1H), 7.786 (d, 1H, J=8.7 Hz), 7.610 (d, 1H, J=2.4 Hz), 7.069 (dd, 1H, J=2.4 & 8.7 Hz), 4.644 (sep, 1H, J=6.0 Hz), 1.393 (d, 6H, J=6.0 Hz).

### (2,5-diisopropoxy-4-methoxyphenyl)(5-isopropoxybenzo[d]thiazol-2-yl)methanol(15)

A solution of 5-isopropoxybenzo[d]thiazole (250 mg, 1 eq) in THF (15 mL) was cooled to −78° C. and n-BuLi (2M in hexane) (1 mL, 1.5 eq) was added slowly dropwise at −78° C. and the reaction mixture was further stirred for 30 min. To the above cooled solution was added 2,5-diisopropoxy-4-methoxybenzaldehyde (391.7 mg, 1.2 eq) in THF (4 mL) at −78° C. The reaction was allowed to warm to room temperature and further stirred for 5 h. The reaction progress was monitored by TLC until analysis indicated complete consumption of starting material. The reaction mixture was quenched with saturated NH₄Cl solution (5 mL), extracted with ethyl acetate (3×15 mL) and washed with brine solution (1×10 mL). The organic layer was dried over anhydrous Na₂SO₄, filtered and evaporated under reduced pressure to obtain crude compound. The crude compound was purified by column chromatography (230-400 mesh silica gel) with 10% ethyl acetate in hexane to afford (2,5-diisopropoxy-4-methoxyphenyl)(5-isopropoxybenzo[d]thiazol-2-yl)methanol (15) as a pale yellow solid; yield 201 mg (35%); ¹H NMR (CDCl₃, 300 MHz) δ 7.660 (d, 1H, J=8.7 Hz), 7.469 (d, 1H, J=2.4 Hz), 6.990 (s, 1H), 6.963 (dd, 1H, J=2.4 & 8.7 Hz), 6.519 (s, 1H), 6.167 (d, 1H, J=6.6 Hz), 4.629-4.482 (m, 2H), 4.386 (sep, 1H, J=6.0 Hz), 4.159 (d, 1H, J=6.0 Hz), 3.836 (s, 3H), 1.376-1.255 (m, 15H), 1.170 (d, 3H, J=6.0 Hz).

### (2,5-diisopropoxy-4-methoxyphenyl)(5-isopropoxybenzo[d]thiazol-yl)methanone (16)

A stirred solution of (2,5-diisopropoxy-4-methoxyphenyl)(5-isopropoxybenzo[d]thiazol-2-yl)methanol (750 mg, 1 eq) in DCM (20 mL) at room temperature was cooled to 0° C. Dess-Martin periodinane (2.85 g, 4 eq) was added at 0° C. and the reaction was stirred for 30 min. The reaction was allowed to warm to room temperature and stirred for 16 hours. The reaction progress was monitored until TLC analysis indicated complete consumption of starting material. The reaction mixture was quenched with saturated NaHCO₃ solution (10 mL), extracted with DCM (2×10 mL) and washed with brine solution (1×10 mL). The organic layer was dried over anhydrous Na₂SO₄, filtered and evaporated under reduced pressure to obtain crude compound. The crude compound was purified by column chromatography (230-400 mesh silica gel) by elution with 10% ethyl acetate in hexane to obtain (2,5-diisopropoxy-4-methoxyphenyl)(5-isopropoxybenzo[d]thiazol-2-yl)methanone (16), yield 550 mg (67%); ¹H NMR (CDCl₃, 300 MHz) δ 7.820 (d, 1H, J=8.7 Hz), 7.574 (d, 1H, J=2.4 Hz), 7.425 (s, 1H), 7.124 (dd, 1H, J=2.4 & 8.7 Hz), 6.530 (s, 1H), 4.582 (sep, 1H, J=6.0 Hz), 4.483-4.392 (m 2H), 3.912 (s, 3H), 1.388-1.364 (m 12H), 1.098 (d, 6H, J=6.0 Hz).

### (2,5-Dihydroxy-4-methoxyphenyl)(5-hydroxybenzo[d]thiazol-2-yl)methanone (FC-6)

A stirred solution of (2,5-diisopropoxy-4-methoxyphenyl)(5-isopropoxybenzo[d]thiazol-2-yl)methanone (550 mg, 1 eq) in DCM (20 mL) at room temperature was cooled to 0° C. AlCl₃ (662 mg, 4 eq) was added slowly at 0° C. and the reaction was stirred for 30 minutes. The reaction mixture was allowed to warm to room temperature and stirred for 16 hours. The reaction progress was monitored until TLC analysis indicated complete consumption of starting material. The reaction mixture was quenched with Conc. HCl and methanol at room temperature, stirred for 30 minutes, and evaporated under reduced pressure to obtain crude compound. The crude compound was purified by prep HPLC to provide (2,5-dihydroxy-4-methoxyphenyl)(5-hydroxybenzo[d]thiazol-2-yl)methanone (FC-6), yield 200 mg (56%); ¹H NMR (DMSO-d6, 400 MHz) δ 8,598 (s, 1H), 8.051 (d, 1H, J=8.76 Hz), 7.583 (d, 1H, J=1.64 Hz), 7.183 (dd, 1H, J=1.64, 8.76 Hz), 6.63 (s, 1H), 3.912 (s, 3H). ¹³C-NMR (DMSO-d6, 100 MHz) δ 184.068, 168.345, 160.696, 157.469, 157.416, 154.748, 139.734, 126.540, 123.080, 118.802, 116.347, 109.945, 108.762, 100.330; HPLC purity, 95.4% (254 nm).

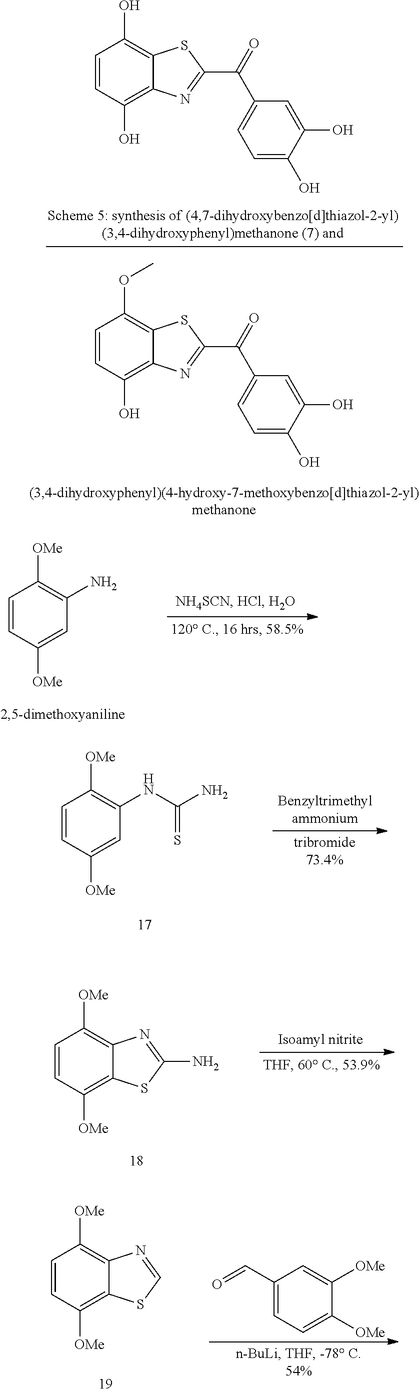

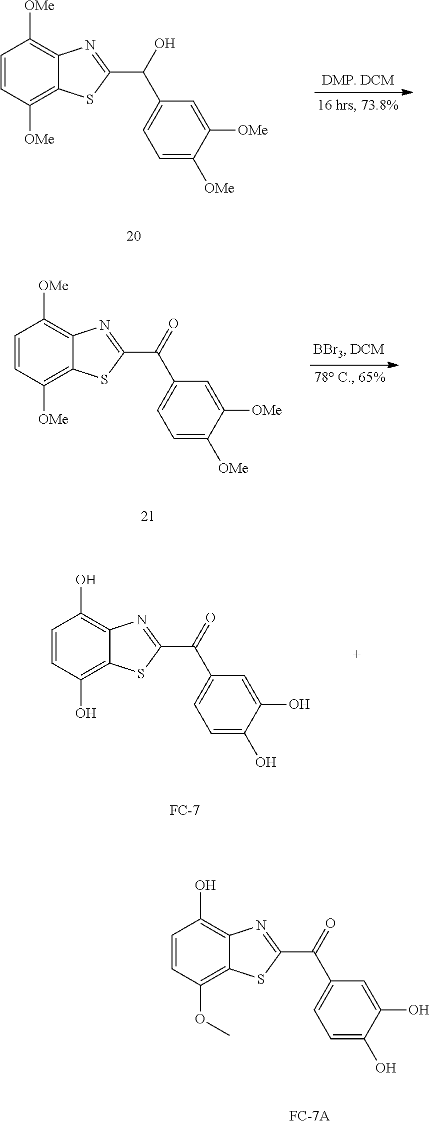 [SMILES: COc1ccc(C(O)c2nc3c(OC)ccc(OC)c3s2)cc1OC ; COc1ccc(C(=O)c2nc3c(OC)ccc(OC)c3s2)cc1OC ; O=C(c1ccc(O)c(O)c1)c1nc2c(O)ccc(O)c2s1 ; "FC-7" ; COc1ccc(O)c2nc(C(=O)c3ccc(O)c(O)c3)sc12 ; "FC-7A"]

### 1-(2,5-Dimethoxyphenyl)thiourea (17)

To a stirred solution of 2,5-dimethoxyaniline (6.8 g, 1 eq) in water (14 mL) and Conc. HCl (7 mL) at room temperature was slowly added NH₄SCN (4.73 g, 4 eq) portionwise at room temperature over a period of 10 minutes. The reaction was heated to 120° C. and stirred for 16 hours. The reaction progress was monitored until TLC analysis indicated complete consumption of starting material. The reaction mixture was diluted with water (100 mL), extracted with ethyl acetate (2×100 mL), and washed with brine solution (1×50 mL). The organic layer was dried over anhydrous Na₂SO₄, filtered and evaporated under reduced pressure to obtain crude compound. The crude compound was purified by column chromatography (230-400 mesh silica gel) by elution with 40% ethyl acetate in hexane to obtain 1-(2,5-dimethoxyphenyl)thiourea (17, 5.5 g) as a white solid, yield 58.5%. ¹H NMR (DMSO-d4, 300 MHz) δ 9.022 (s 1H), 6.934 (dd, 1H, J=9, 4.2 Hz), 6.669 (dd, 1H, J=9.3 Hz), 3.765 (s, 3H), 3.691 (d, 3H, J=5.7 Hz),

### 4,7-Dimethoxybenzo[d]thiazol-2-amine (18)

To a stirred solution of 1-(2,5-dimethoxyphenyl)thiourea (5.5 g, 1 eq) in acetic acid (160 mL) at room temperature was slowly added dropwise benzyltrimethylammonium tribromide (11.12 g, 1.1 eq) at room temperature over a period of 10 minutes. The reaction was stirred for 1 hour at room temperature. The reaction progress was monitored by TLC analysis until complete consumption of starting material was indicated. The reaction mixture was filtered and washed with acetic acid (10 mL) to obtain crude solid compound. The solid was dissolved in saturated NaHCO₃ solution (100 mL), extracted with ethyl acetate (2×100 mL) and washed with brine solution (1×50 mL). The organic layer was dried over anhydrous Na₂SO₄, filtered and evaporated under reduced pressure to obtain 4,7-dimethoxybenzo[d]thiazol-2-amine (18, 4 g) as a white solid, yield 73.4%. ¹H NMR (CDCl₃, 300 MHz) δ 6.737 (d, 1H, J=8.7 Hz), 6.550 (d, 1H, J=8.7 Hz), 5.289 (br s, 2H), 3.932 (s, 3H), 3.877 (s, 3H).

### 4,7-Dimethoxybenzo[d]thiazole (19)

To a stirred solution of 4,7-dimethoxybenzo[d]thiazol-2-amine (6 g, 1 eq) was added isoamyl nitrite (5.6 g, 2.5 eq) in THF (60 mL) at room temperature. The reaction was heated to 60° C. and stirred for 3 hours. The reaction progress was monitored by TLC analysis until complete consumption of starting material was indicated. The reaction mixture was diluted with water (100 mL), extracted with ethyl acetate (2×100 mL), and washed with brine solution (1×50 mL). The organic layer was dried over anhydrous Na₂SO₄, filtered and evaporated under reduced pressure to obtain crude compound. The crude compound was purified by column chromatography (230-400 mesh silica gel) eluting with 20% ethyl acetate in hexane to obtain 4,7-dimethoxybenzo[d]thiazole (19, 3 g) as a red solid, yield 53.9%. ¹H NMR (CDCl₃, 300 MHz) δ 8.907 (s, 1H), 6.858 (d, 1H, J=8.4 Hz), 6.787 (d, 1H, J=8.4 Hz), 4.027 (s, 3H), 3.959 (s, 3H)

### (4,7-Dimethoxybenzo[d]thiazol-2-yl)(3,4-dimethoxyphenyl)methanol (20)

A stirred solution of 4,7-dimethoxybenzo[d]thiazole (1.5 g, 1 eq) in THF (100 mL) was cooled to −78° C. followed by slow dropwise addition of n-BuLi (2M in Hexane) (738 mg, 1.5 eq) at −78° C., and stirred for 30 minutes. Next, 3,4-dimethoxybenzaldehyde (1.53 g, 1.2 eq) was added at −78° C. The reaction was allowed to warm to room temperature and stirred for 1 hour. The reaction progress was monitored by TLC analysis until complete consumption of starting material was indicated. The reaction mixture was quenched with saturated NH₄Cl solution (20 mL), extracted with ethyl acetate (3×50 mL) and washed with brine solution (1×20 mL). The organic layer was dried over anhydrous Na₂SO₄, filtered and evaporated under reduced pressure to obtain crude compound. The crude compound was purified by column chromatography (230-400 mesh silica gel) eluting with 50% ethyl acetate in hexane to afford (4,7-dimethoxybenzo[d]thiazol-2-yl)(3,4-dimethoxyphenyl)methanol (20, 1.5 g) as a yellow solid, yield 54%. ¹H NMR (CDCl₃, 300 MHz) δ 7.104-7.072 (m, 2H), 6.855 (d, 1H, J=8.1 Hz), 6.823 (d, 1H, J=8.4 Hz), 6.725 (d, 1H, J=8.7 Hz), 6.131 (d, 1H, J=2.7 Hz), 4.005 (s, 3H), 3.903 (s, 3H), 3.872 (s, 3H), 3.864 (s, 3H), 3.488 (d, 1H, J=3.0 Hz).

### (4,7-Dimethoxybenzo[d]thiazol-2-yl)(3,4-dimethoxyphenyl)methanone (21)

A stirred solution of (4,7-dimethoxybenzo[d]thiazol-2-yl)(3,4-dimethoxyphenyl)methanol (1.5 g, 1 eq) in DCM (150 mL) was cooled to 0° C. Added Dess-Martin periodinane (7.5 g, 4 eq) at 0° C. and stirred for 30 minutes. The reaction was allowed to warm to room temperature and stirred for 16 hours. The reaction progress was monitored by TLC analysis until complete consumption of starting material was indicated. The reaction mixture was quenched with saturated NaHCO₃ solution (10 mL), extracted with DCM (2×50 mL) and washed with brine solution (1×20 mL). The organic layer was dried over anhydrous Na₂SO₄, filtered and evaporated under reduced pressure to obtain crude compound. The crude compound was purified by column chromatography (230-400 mesh silica gel) eluting with 20% ethyl acetate in hexane to obtain (4,7-dimethoxybenzo[d]thiazol-2-yl)(3,4-dimethoxyphenyl) methanone (21, 1.1 g) as a yellow solid, yield 73.8% . ¹H NMR (CDCl₃, 300 MHz) δ 8.545 (dd, 1H, J=2.1, 8.7 Hz), 8.091 (d, 1H, J=2.1 Hz), 7.015 (d, 1H, J=8.4 Hz), 6.895 (d, 1H, J=8.7 Hz), 6.862 (d, 1H, J=8.4 Hz), 4.050 (s, 3H), 3.996 (s, 6H), 3.982 (s, 3H).

### (4,7-Dihydroxybenzo[d]thiazol-2-yl)(3,4-dihydroxyphenyl)methanone (FC-7&7A)

To a solution of (4,7-dimethoxybenzo[d]thiazol-2-yl)(3,4-dimethoxyphenyl)methanone (600 mg, 1 eq) in DCM (15 mL) at −78° C. was slowly added BBr₃ (1M in DCM, 16.7 mL, 10 eq) at 0° C. and the reaction was stirred for 30 min. The reaction mixture was allowed to warm to room temperature and stirred for 16 h. The reaction progress was monitored by TLC: analysis indicated >50%* consumption of starting material. The reaction mixture was quenched with methanol at 0° C., stirred for 30 min, and evaporated under reduced pressure to obtain crude compound. The crude compound was purified by column chromatography over silica gel (230-400 mesh) using 5% MeOH in DCM to afford (3,4-dihydroxyphenyl)(4-hydroxy-7-methoxybenzo[d]thiazol-2-yl)methanone (FC-7A, 100 mg) as a yellow solid, yield 18.9% and (4,7-dihydroxybenzo[d]thiazol-2-yl)(3,4-dihydroxyphenyl)methanone (FC-7, 250 mg) as a brown color solid, yield 47.3%.

FC-7A: ¹H NMR (DMSO-d6, 500 MHz) δ 8.126 (dd, 1H, J=1.9, 8.4 Hz), 7.885 (d, 1H, J=1.9 Hz), 7.01-6.93 (m, 3H), 3.958 (s, 3H); ¹³C-NMR (DMSO-d6, 125 MHz) δ 182.202, 166.187, 152.441, 148.102, 145.460, 145.371, 144.530, 125.858, 125.806, 125.332, 117.606, 115.406, 112.929, 109.040, 56.254. HPLC purity, 96.1% (254 nm).

FC-7: ¹H NMR (DMSO-d6, 500 MHz) δ 8.259 (dd, 1H, J=1.6, 6.8 Hz), 7.87 (d, 1H, J=1.6 Hz), 6.923 (d, 1H, J=8.4 Hz), 6.830 (s, 2H); ¹³C-NMR (DMSO-d6, 125 MHz) δ 182.085, 165.368, 152.393, 146.264, 145.389, 144.217, 144.163, 125.914, 125.821, 125.041, 117.491, 115.329, 112.839, 112.203. HPLC purity, 97.3% (254 nm).

FC-7&7A were purified and characterized separately. The position of the methoxy group in FC-7A was confirmed by NOE difference spectra.

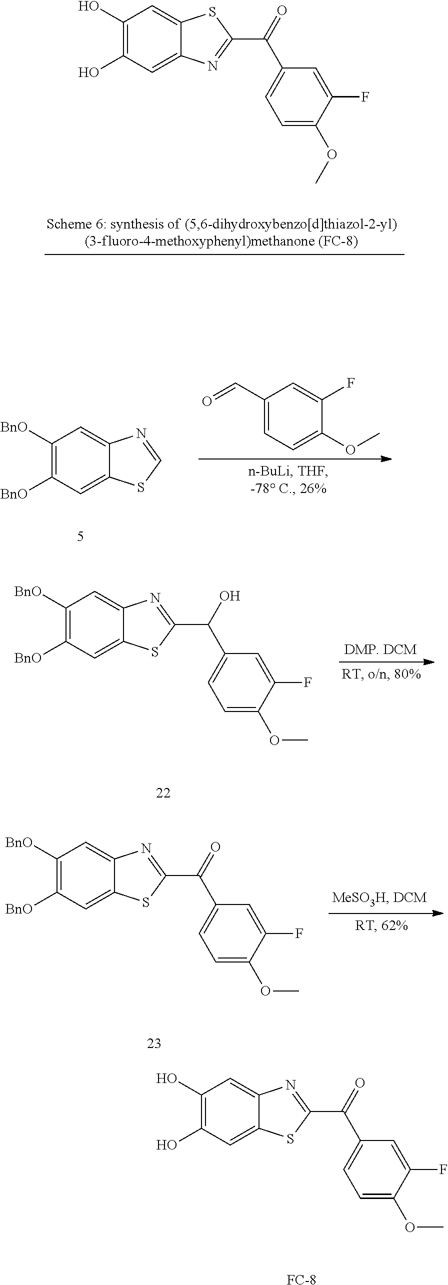

### (5,6-Bis(benzyloxy)benzo[d]thiazol-2-yl)(3-fluoro-4-methoxyphenyl)methanol (22)

To a stirred solution of 5,6-bis(benzyloxy)benzo[d]thiazole (1 g, 1 eq) in THF (40 mL) −78° C. was added slowly n-BuLi (2M in hexane, 2.2 mL, 1.5 eq) and the reaction was stirred for 30 min. To the above solution was added 3-fluoro-4-methoxybenzaldehyde (554 mg, 1.25 eq) in THF (10 mL) at −78° C. The reaction mixture was allowed to warm to room temperature and further stirred overnight. The reaction progress was monitored by TLC until analysis indicated complete consumption of starting material. The reaction mixture was quenched by addition of saturated NH₄Cl solution (15 mL), extracted with ethyl acetate (2×50 mL) and washed with brine solution (1×10 mL). The organic layer was dried over anhydrous Na₂SO₄, filtered and evaporated under reduced pressure to obtain crude compound. The crude compound was purified by column chromatography (230-400 mesh silica gel) with 15% ethyl acetate in hexane to obtain (5,6-bis(benzyloxy)benzo[d]thiazol-2-yl)(3-fluoro-4-methoxyphenyl)methanol (22) as a pale yellow solid; yield 380 mg (26%); ¹H NMR (CDCl₃, 300 MHz), δ 7.495-7.208 (m, 14H), 6.919 (t, 1H, J=8.4 Hz), 6.006 (br d, 1H), 5.197 (s, 2H), 5.187 (s, 2H), 3.859 (s, 3H).

### (5,6-Bis(benzyloxy)benzo[d]thiazol-2-yl)(3-fluoro-4-methoxyphenyl)methanone (23)

To a solution of (5,6-bis(benzyloxy)benzo[d]thiazol-2-yl)(3-fluoro-4-methoxyphenyl) methanol (380 mg, 1 eq) in DCM (10 mL) at 0° C. was added Dess-Martin Periodinane (DMP, 965 mg, 3 eq) and the reaction was stirred for 30 min. The reaction mixture was allowed to warm to room temperature and stirred for 16 h. The reaction progress was monitored by TLC until analysis indicated complete consumption of starting material. The reaction mixture was quenched with saturated NaHCO₃ solution (10 mL), extracted with DCM (2×20 mL) and washed with brine solution (1×20 mL). The organic layer was dried over anhydrous Na₂SO₄, filtered and evaporated under reduced pressure to obtain crude compound. The crude compound was purified by column chromatography (230-400 mesh silica gel) eluting with 15% ethyl acetate in hexane to obtain (5,6-bis(benzyloxy)benzo[d] thiazol-2-yl)(3-fluoro-4-methoxyphenyl)methanone (23) as a pale yellow solid; yield 301 mg (80%); ¹H NMR (CDCl₃, 300 MHz), δ8.465-8.408 (m, 2H), 7.705 (s, 1H), 7.521-7.482 (m. 4H), 7.426-7.331 (m, 7H), 7.086 (t, 1H, J=8.7 Hz), 5.292 (s, 2H), 5.286 (s, 2H), 4.001 (s, 3H).

### (5,6-Dihydroxybenzo[d]thiazol-2-yl)(3-fluoro-4-methoxyphenyl)methanone (FC-8)

To a stirred solution of (5,6-bis(benzyloxy)benzo[d]thiazol-2-yl)(3-fluoro-4-methoxyphenyl)-methanone (300 mg, 1 eq) in DCM (10 mL) at 0° C. was added methanesulfonic acid (4 mL) and the reaction was stirred for 30 min. The reaction mixture was allowed to warm to room temperature and further stirred for 2 h. The reaction progress was monitored by TLC analysis until complete consumption of starting material was indicated. The reaction mixture was diluted with DCM (20 mL), washed with saturated NaHCO₃ solution (1×10 mL), water (1×10 mL). The organic layer was dried over anhydrous Na₂SO₄, filtered and evaporated under reduced pressure to obtain crude compound. The crude compound was purified by prep HPLC to obtain (5,6-dihydroxybenzo[d]thiazol-2-yl)(3-fluoro-4-methoxyphenyl)methanone (FC-8); yield 120 mg (62%); ¹H NMR (DMSO-d6, 500 MHz) δ 8.427 (d, 1H, J=9.4 Hz), 8.314 (dd, 1H, J=1.9, 12.5 Hz), 7.527 (s, 1H), 7.465 (s, 1H), 7.406 (t, 1H, J=8.7 Hz), 3.980 (s, 3H); ¹³C-NMR (DMSO-d6, 125 MHz) δ 182.085, 165.368, 152.393, 146.264, 145.389, 144.217, 144.163, 125.914, 125.821, 125.041, 117.491, 115.329, 112.839, 112.203. HPLC purity, 95.6% (254 nm).

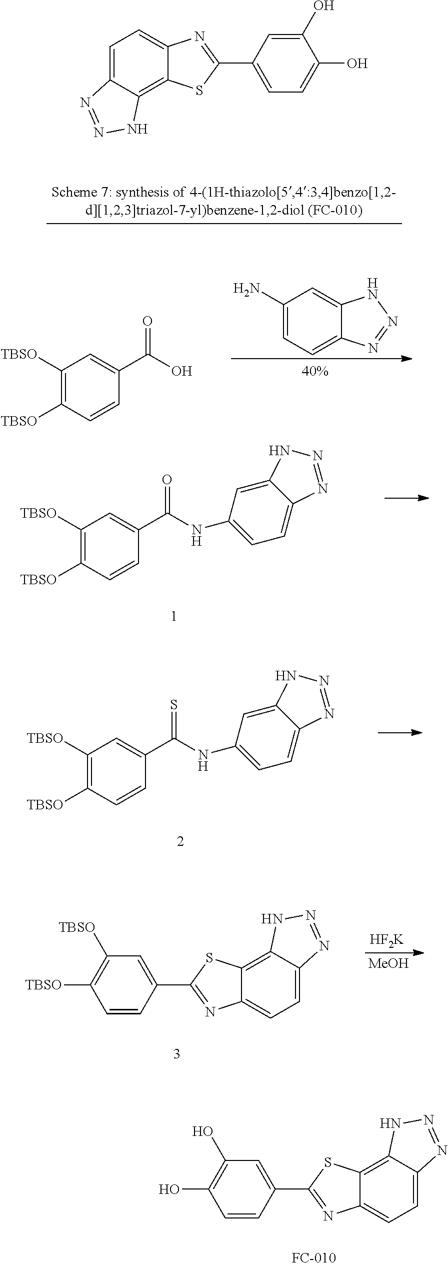

N-(1H-benzo[d][1,2,3]triazol-6-yl)-3,4-bis((tert-butyldimethylsilyl)oxy)benzamide (1). To a solution of the benzoic acid (382 mg, 1.0 mmol) in DCM (5.0 mL) was added three drops of DMF and thionyl chloride SOCl₂ (1.0 mL). The reaction mixture was stirred at RT for 45 min. The volatiles were removed under vacuum. After drying under high vacuum for one h, the resulting benzoyl chloride residue was dissolved in DCM (10.0 mL) and cooled down to 0° C. To the stirred solution at 0° C. was added 6-amino benzotriazole (134 mg, 1.0 mmol), and DIPEA (700 uL, 4.0 mmol). The reaction mixture was stirred at RT for four h at which time the organic layer was transferred to a separatory funnel and was washed with a saturated solution of NaHSO₄ (10 mL), saturated solution of NaHCO₃, and brine. Then, the organic layer was dried over Na₂SO₄, filtered, and evaporated under vacuum to give a crude anilide, which was purified by flash chromatography (FCC) using a gradient Hexanes/EtOAc solvent system. FCC purification gave 200 mg 1, 40%). N-(1H-benzo[d][1,2,3]triazol-6-yl)-3,4-bis((tert-butyldimethylsilyl)oxy)benzothioamide (2) To a solution of the anilide 1 (195 mg, 0.39 mmol) in Toluene (3.0 mL) was added Lawesson's reagent (95 mg, 0.234 mmol). The reaction mixture was refluxed at 120° C. for three h at which time the toluene was evaporated to dryness under vacuum. The resulting crude mixture was loaded onto a silica gel matrix and was purified using flash chromatography using a gradient solvent of Hexanes/EtOAc to afford 70 mg of the thioanilide 2 and 110 mg recovered starting material (80% yield).

7-(3,4-bis((tert-butyldimethylsilyl)oxy)phenyl)-1H-thiazolo[5′,4′:3,4]benzo[1,2-d][1,2,3]triazole (3). To a solution of the thioanilide 2 (70 mg, 0.136 mmol) in CHCl3 (2.0 mL) was added TEMPO (42 mg, 0.272 mmol). The reaction mixture was stirred at RT under the direct exposure of white light for 16 h at which time the excess solvent was evaporated under vacuum, and the resulting crude mixture was subjected to FCC purification using 95% CHCl₃/MeOH to furnish the corresponding benzothiazole 3 (% yield). LRMS (ESI, MH+) m/z calc for C25H37N4O2SSiH+513.22, found 513.4.

4-(1H-thiazolo[5′,4′:3,4]benzo[1,2-d][1,2,3]triazol-7-yl)benzene-1,2-diol (FC-010). To a solution of 3 (20 mg, 0.04 mmol) in MeOH (1 mL) was added HF2K (16 mg, 0.2 mmol). The reaction mixture was stirred at RT for 3 h or until the disappearance the starting material. Upon completion the volatiles were removed under vacuum and the residue was loaded onto a silica column and purified using 10% MeOH/DCM. ¹HNMR (500 MHz, DMSO-d₆) δ9.78 (s, 1H), 9.56 (s, 1H), 8.05 (bs, 1H), 7.90 (bs, 1H), 7.57 (d, J=2.1 Hz, 1H), 7.47 (dd, J=2.1, 8.15 Hz, 1H), 6.92 (d, J=8.2 Hz, 1H). ¹³C NMR (125 MHz, DMSOd₆) δ. 149.0, 146.0, 124.1, 119.4, 116.3, 113.9; HPLC purity 92.5% (254 nm).

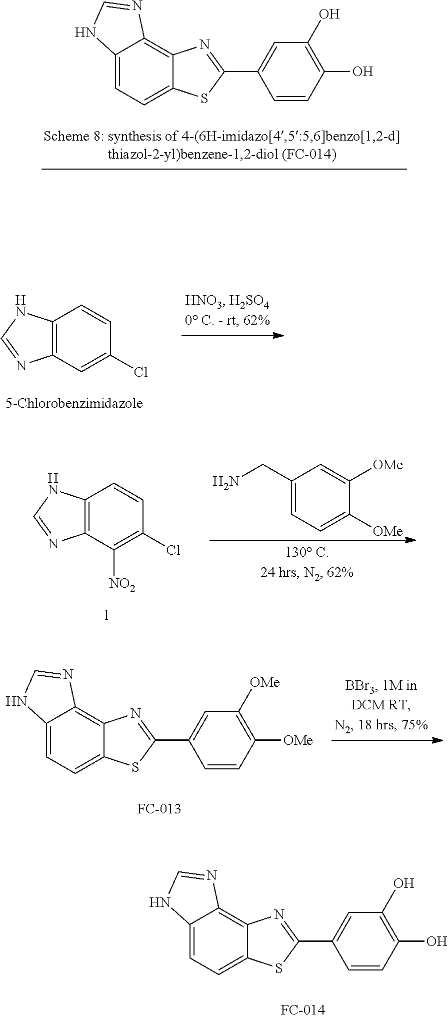 [SMILES: Oc1ccc(-c2nc3c(ccc4[nH]cnc43)s2)cc1O ; Clc1ccc2[nH]cnc2c1 ; O=[N+]([O-])c1c(Cl)ccc2[nH]cnc12 ; COc1ccc(CN)cc1OC ; COc1ccc(-c2nc3c(ccc4[nH]cnc43)s2)cc1OC ; Oc1ccc(-c2nc3c(ccc4[nH]cnc43)s2)cc1O]

5-Chloro-4-nitro-1H-benzoimidazole (1). 5-Chlorobenzimidazole (3.3 mmol) was added to a cooled solution of concentrated sulfuric acid (3 mL) and fuming nitric acid (3 mL) in an ice-bath with stirring. The reaction mixture was stirred at room temperature for 1 h and then poured into ice-water. The precipitate was collected and dried. The crude solid was purified by FCC to give the corresponding compound (403 mg, 62%). ESI mass spectroscopy (MH+=198).

2-(3,4-Dimethoxy-phenyl)-6H-imidazo[4′,5′:3,4]benzo[2,1-d]thiazole (FC-013). A mixture of compound (1) (0.5 mmol), 3,4-Dimethoxybenzylamine (1.5 mmol), and elemental sulfur (1.0 mmol) was stirred in a sealed tube under nitrogen atmosphere at 130° C. for 24 h. After cooling to room temperature, the crude mixture was triturated and dissolved in EtOAc. The mixture was filtered, the filtrate was concentrated, and the crude residue was purified by FCC on silica gel to give the corresponding compound FC-013 (96 mg, 62%). ¹HNMR (500 MHz, DMSO-d6) δ 13.09 (br, 1H), 8.31 (s, 1H), 7.85 (d, J=7.1 Hz, 1H), 7.66 (m, 3H), 7.14 (d, J=7.0 Hz, 1H), 3.92 (s, 3H), 3.86 (s, 3H); ¹³C NMR (125 MHz, DMSO-d6) δ 167.36, 151.83, 149.64, 141.64, 128.88, 126.60, 121.04, 115.52, 112.55, 109.93, 56.22, 56.15. ESI mass spectroscopy (MH+=312); HPLC purity, 95.6% (254 nm). 4-(6H-imidazo[4′,5′:5,6]benzo[1,2-d]thiazol-2-yl)benzene-1,2-diol (FC-014)

A solution of FC-013 (0.13 mmol in 0.5 mL dry dichloromethane) was treated with an excess (8-10 eq) of boron tribromide (1M solution in dichloromethane) at 0° C. under N₂. The reaction mixture was allowed to reach room temperature over 18 h and then quenched with aqueous saturated NaHCO₃. The mixture was stirred for ½ h and then diluted with water and EtOAc. The organic layer was separated from aqueous. The aqueous portion was neutralized with 1N HCl to pH 4 and extracted with EtOAc. The combined EtOAc extracts were washed with brine and dried with anhydrous MgSO4. The crude residue was purified by FCC on silica gel to give the corresponding compound FC-014 (27 mg, 75%). ¹HNMR (500 MHz, DMSO-d6) δ 9.70 (s, 1H), 9.50 (s, 1H), 8.61 (s, 1H), 7.95 (d, J=7.1 Hz, 1H), 7.73 (d, J=7.1 Hz, 1H), 7.60 (d, J=1.5 Hz, 1H), 7.47 (dd, J=6.9, 1.6 Hz, 1H), 6.94 (d, J=6.9 Hz, 1H); 13C NMR (125 MHz, DMSO-d6) δ 168.11, 148.81, 145.81, 142.22, 142.18, 140.82, 135.7, 128.85, 119.23, 116.06, 113.89; ESI mass spectroscopy (MH+=284); 163.0681; HPLC purity, 92.8% (254 nm).

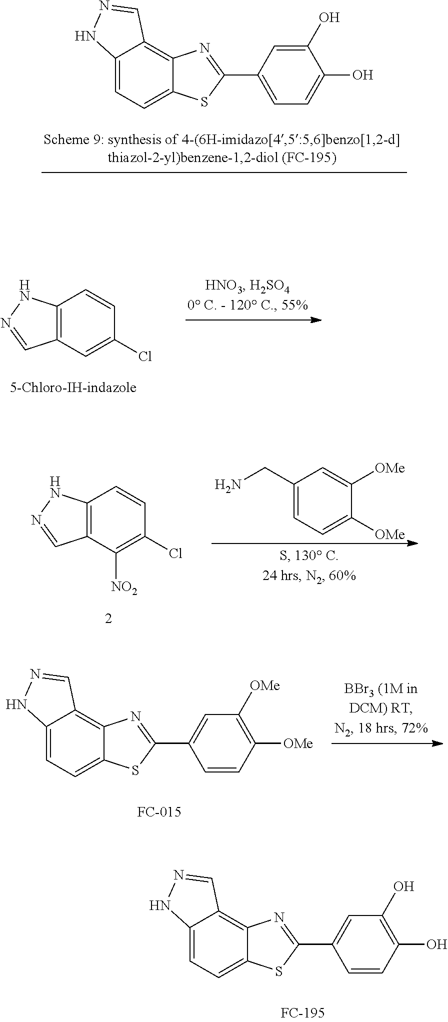 [SMILES: Oc1ccc(-c2nc3c(ccc4[nH]ncc43)s2)cc1O ; Clc1ccc2[nH]ncc2c1 ; O=[N+]([O-])c1c(Cl)ccc2[nH]ncc12 ; COc1ccc(CN)cc1OC ; COc1ccc(-c2nc3c(ccc4[nH]ncc43)s2)cc1OC ; Oc1ccc(-c2nc3c(ccc4[nH]ncc43)s2)cc1O]

5-Chloro-4-nitro-1H-indazole (2). 5-Chloro-1H-indazole (6.5 mmol) was added to a cooled solution of concentrated sulfuric acid (5 mL) and fuming nitric acid (5 mL) in an ice-bath with stirring. The reaction mixture was stirred at room temperature for ½ h, followed by heating at 120° C. for 1 h and then poured into ice-water. The precipitate was collected and dried. The crude solid was purified by FCC to give the corresponding compound (704 mg, 55%). ¹HNMR (500 MHz, Acetoned₆) δ 8.19 (s, 1H), 7.69 (d, J=8.2 Hz, 1H), 7.55 (d, J=7.1 Hz, 1H); ¹³C NMR (125 MHz, Acetoned₆) δ141.74, 132.32, 130.19, 120.73, 117.70, 114.22. ESI mass spectroscopy (MH⁺=198).

2-(3,4-dimethoxyphenyl)-6H-thiazolo[4,5-e]indazole (FC-015). A mixture of compound (2) (0.5 mmol), 3,4-Dimethoxybenzylamine (1.5 mmol), and elemental sulfur (1.0 mmol) was stirred in a sealed tube under nitrogen atmosphere at 130° C. for 24 h. After being cooled to room temperature, the crude mixture was triturated and dissolved in EtOAc. The mixture was filtered and concentrated, and the crude residue was purified by FCC on silica gel to give the corresponding compound FC-015 (93 mg, 60%). ¹HNMR (500 MHz, DMSO-d₆) δ 13.49 (br, 1H), 8.48 (s, 1H), 8.03 (d, J=7.3 Hz, 1H), 7.68 (m, 3H), 7.17 (d, J=6.7 Hz, 1H), 3.96 (s, 3H), 3.90 (s, 3H); ¹³C NMR (125 MHz, DMSO-d₆) δ 168.86, 152.29, 150.06, 146.74, 127.14, 126.86, 121.37, 120.14, 117.60, 112.96, 110.42, 56.63, 56.61. ESI mass spectroscopy (MH⁺=312); HPLC purity, 94.9% (254 nm).

4-(6H-imidazo[4′,5′:5,6]benzo[1,2-d]thiazol-2-yl)benzene-1,2-diol (FC-195). A solution of FC-015 (0.063 mmol in 0.25 mL dry dichloromethane) was treated with an excess (8-10 eq) of boron tribromide (1M solution in dichloromethane) at 0° C. under N₂. The reaction mixture was allowed to reach room temperature over 18 h and then quenched with aqueous saturated NaHCO₃. The mixture was stirred for ½ h and then diluted with water and EtOAc. The organic layer was separated from aqueous. The aqueous portion was neutralized with 1N HCl to pH 4 and extracted with EtOAc. The combined EtOAc extracts were washed with brine and dried with anhydrous MgSO₄. The crude residue was purified by FCC on silica gel to give the corresponding compound (13 mg, 72%). ¹HNMR (500 MHz, DMSO-d₆) δ 13.44 (s, 1H), 9.67 (s, 1H), 9.50 (s, 1H), 8.39 (s, 1H), 7.97 (d, J=8.75 Hz, 1H), 7.59 (m, 2H), 7.43 (dd, J₁=8.2 Hz, J₂=2.1 Hz, 1H), 6.88 (d, J=8.2 Hz, 1H); ¹³C NMR (125 MHz, DMSO-d₆) δ 168.53, 145.85, 140.06, 131.26, 125.87, 124.67, 119.61, 119.13, 116.71, 116.20, 113.72, 108.49. ESI mass spectroscopy (MH⁺=284). HPLC purity, 90.8% (254 nm).

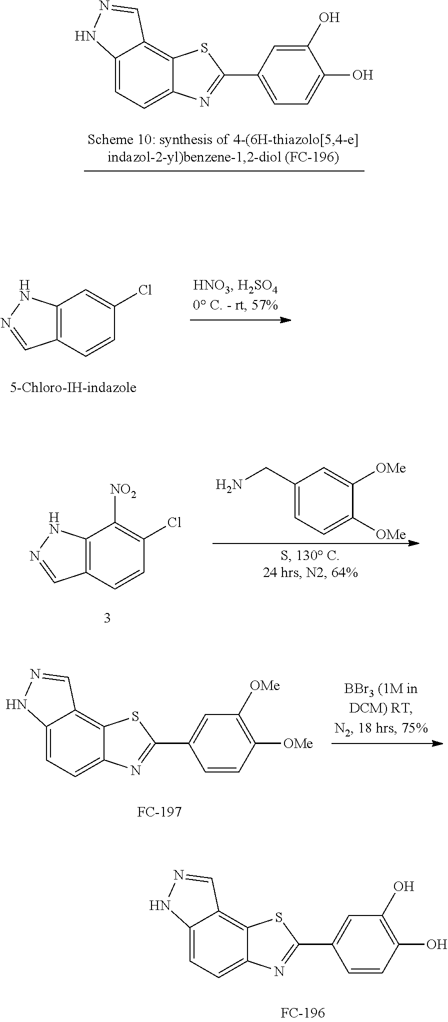 [SMILES: Oc1ccc(-c2nc3ccc4[nH]ncc4c3s2)cc1O ; Clc1ccc2cn[nH]c2c1 ; O=[N+]([O-])c1c(Cl)ccc2cn[nH]c12 ; COc1ccc(CN)cc1OC ; COc1ccc(-c2nc3ccc4[nH]ncc4c3s2)cc1OC ; Oc1ccc(-c2nc3ccc4[nH]ncc4c3s2)cc1O]

6-chloro-7-nitro-1H-indazole (3). 5-Chlorobenzimidazole (3.3 mmol) was added to a cooled solution of concentrated sulfuric acid (3 mL) and fuming nitric acid (3 mL) in an ice-bath with stirring. The reaction mixture was stirred at room temperature for 1 h and was poured into ice-water. The precipitate was collected and dried. The crude solid was purified by FCC to give the corresponding compound (370 mg, 57%). ESI mass spectroscopy (MH+=198).

2-(3,4-dimethoxyphenyl)-6H-thiazolo[5,4-e]indazole (FC-197). A mixture of compound (3) (1 mmol), 3,4-Dimethoxybenzylamine (3.0 mmol), and elemental sulfur (2.0 mmol) was stirred in a sealed tube under nitrogen atmosphere at 130° C. for 24 h. After cooling to room temperature, the crude mixture was triturated and dissolved in EtOAc. The mixture was filtered and concentrated, and the crude residue was purified by FCC on silica gel to give the corresponding compound (200 mg, 64%). ¹HNMR (600 MHz, DMSO-d₆) δ 7.80 (s, 1H), 7.75 (m, 2H), 7.67 (m, 2H), 7.15 (d, J=8.4 Hz, 1H), 3.91 (s, 3H), 3.86 (s, 3H); ¹³C NMR (125 MHz, DMSO-d₆) δ 166.83, 151.36, 149.14, 138.41, 134.42, 133.93, 132.11, 125.79, 122.29, 120.58, 118.17, 114.19, 112.02, 109.35, 55.73, 55.67. ESI mass spectroscopy (MH⁺=312). HPLC purity, 91.1% (254 nm).

4-(6H-thiazolo[5,4-e]indazol-2-yl)benzene-1,2-diol (FC-196). To a solution of FC-197 (0.16 mmol in 0.5 mL dry dichloromethane) was treated with an excess (8-10 eq) of boron tribromide (1M solution in dichloromethane) at 0° C. under N₂. The reaction mixture was allowed to reach room temperature over 18 h and then quenched with aqueous saturated NaHCO₃. The mixture was stirred for ½ h and then diluted with water and EtOAc. The organic layer was separated from aqueous. The aqueous portion was neutralized with 1N HCl to pH 4 and extracted with EtOAc. The combined EtOAc extracts were washed with brine and dried with anhydrous MgSO₄. The crude residue was purified by FCC on silica gel to give the corresponding compound (34 mg, 75%). ¹HNMR (500 MHz, DMSO-d₆). δ 13.93 (br, 1H), 9.72 (s, 1H), 9.52 (s, 1H), 8.21 (s, 1H), 7.74 (m, 2H), 7.55 (d, J-2.1 Hz, 1H), 7.42 (dd, J₁=8.2 Hz, J₂=2.1 Hz, 1H), 6.90 (d, J=8.2 Hz, 1H); ¹³C NMR (125 MHz, DMSO-d₆) δ 167.32, 148.77, 145.88, 138.39, 134.60, 133.76, 131.78, 124.58, 122.28, 119.18, 117.82, 116.20, 114.20, 113.92. ESI mass spectroscopy (MH⁺=284). HPLC purity, 90.2% (254 nm).

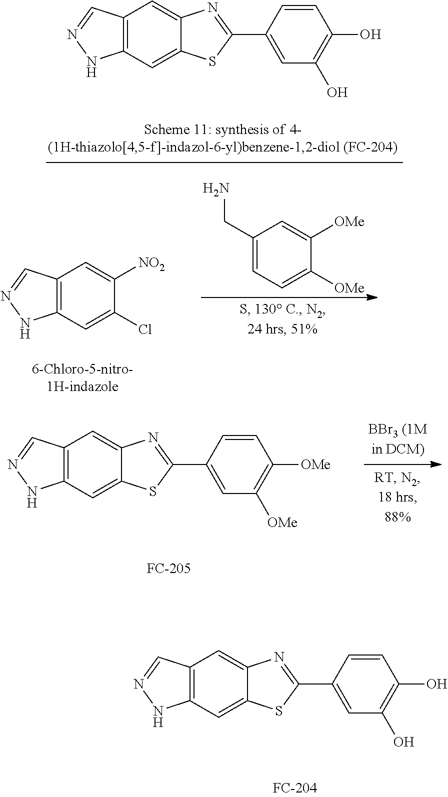 [SMILES: Oc1ccc(-c2nc3cc4cn[nH]c4cc3s2)cc1O ; O=[N+]([O-])c1cc2cn[nH]c2cc1Cl ; COc1ccc(CN)cc1OC ; COc1ccc(-c2nc3cc4cn[nH]c4cc3s2)cc1OC ; Oc1ccc(-c2nc3cc4cn[nH]c4cc3s2)cc1O]

6-(3,4-dimethoxyphenyl)-1H-thiazolo[4,5-f]-indazole (FC-205). A mixture of 6-Chloro-5-nitro-1H-indazole (1 mmol), 3,4-Dimethoxybenzylamine (3.0 mmol), and elemental sulfur (2.0 mmol) was stirred in a sealed tube under nitrogen atmosphere at 130° C. for 24 h. After cooling to room temperature, the crude mixture was triturated and dissolved in EtOAc. The mixture was filtered and concentrated, and the crude residue was purified by FCC on silica gel to give the corresponding compound (160 mg, 51%). ¹HNMR (600 MHz, DMSO-d₆) δ 13.14 (br, 1H), 8.38 (s, 1H), 8.24 (s, 1H), 8.20 (s, 1H), 7.65 (d, J=1.9 Hz, 1H), 7.58 (dd, J₁=8.3 Hz, J₂=2.0 Hz, 1H), 7.13 (d, J=8.4 Hz, 1H), 3.90 (s, 3H), 3.86 (s, 3H); ¹³C NMR (125 MHz, DMSO-d₆) δ 165.27, 151.49, 149.04, 148.95, 138.37, 134.45, 134.13, 125.70, 123.27, 120.95, 112.97, 111.81, 109.16, 102.02, 55.67, 55.56. ESI mass spectroscopy (MH⁺=312). HPLC purity, 94.38% (254 nm).

4-(1H-thiazolo[4,5-f]-indazol-6-yl)benzene-1,2-diol (FC-204). A solution of FC-205(0.1 mmol in 0.5 mL dry dichloromethane) was treated with an excess (8-10 eq) of boron tribromide (1M solution in dichloromethane) at 0° C. under N₂. The reaction mixture was allowed to reach room temperature over 18 h and then quenched with aqueous saturated NaHCO₃. The mixture was stirred for ½ h and then diluted with water and EtOAc. The organic layer was separated from aqueous. The aqueous portion was neutralized with 1N HCl to pH 4 and extracted with EtOAc. The combined EtOAc extracts were washed with brine and dried with anhydrous MgSO₄. The crude residue was purified by HPLC to give the corresponding compound (24 mg, 88%). ¹HNMR (500 MHz, DMSO-d₆). δ 13.11 (s, 1H), 9.65 (s, 1H), 9.44 (s, 1H), 8.31 (s, 1H), 8.22 (s, 1H), 8.17 (s, 1H), 7.52 (d, J=2.0 Hz, 1H), 7.37 (dd, J₁=8.2 Hz, J₂=2.1 Hz, 1H), 6.89 (d, J=8.2 Hz, 1H); ¹³C NMR (125 MHz, DMSO-d₆) δ 165.61, 149.09, 148.90, 145.75, 138.26, 134.38, 134.07, 124.47, 123.18, 119.46, 116.06, 11389, 112.61, 101.92. ESI mass spectroscopy (MH⁺=284). HPLC purity, 97.1% (254 nm)

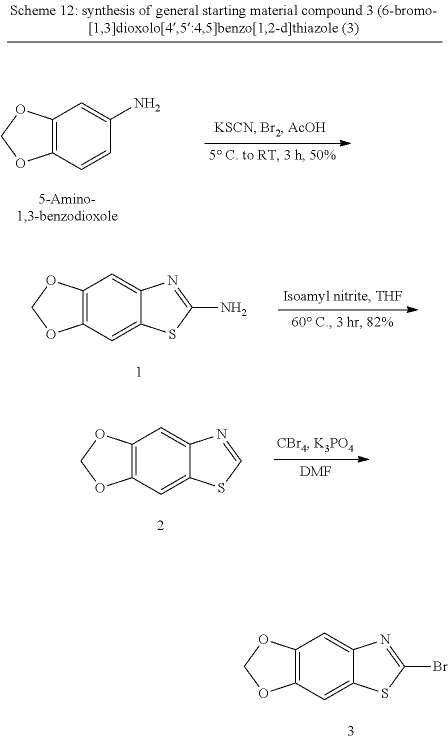

[1,3]Dioxolo[4′,5′:4,5]benzo[1,2-d]thiazol-6-amine (1). To a stirred solution of 5-Amino-1,3-benzodioxole (1 g, 1 eq), KSCN (2.8 g, 4 eq) in acetic acid (10 mL) at 0° C., bromine (1.17 g, 1 eq) in acetic acid (10 mL) was added dropwise at 0 to 5° C. over a period of 30 min. The reaction was allowed to warm to room temperature and further stirred for 2 h. The reaction progress was monitored until TLC analysis indicated complete consumption of starting material. The reaction mixture was diluted with water (100 mL), saponified with aqueous ammonia solution (50 mL), and stirred for 30 min. The solid obtained was filtered and washed with water (1×20 mL) to get crude solid compound. The crude compound was purified by column chromatography (230-400 mesh silica gel, eluent 2% methanol in DCM) to afford [1,3]dioxolo[4′,5′:4,5]benzo[1,2-d]thiazol-6-amine (1) as pale green solid; yield 0.72 g (50%); ¹H NMR (DMSO-d6, 300 MHz) δ 7.251 (s, 1H), 7.192 (s, 2H), 6.924 (s, 1H), 5.958 (s, 2H).

To a stirred solution of compound 1 (1 g, 1 eq), in THF (10 mL) was added isoamyl nitrite (1.3 g, 2.5 eq) at room temperature. The reaction mixture was heated to 60° C. and stirred for 3 h. The reaction progress was monitored by TLC until analysis indicated complete consumption of starting material. The reaction mixture was diluted with ethyl acetate (100 mL), washed with water (1×25 mL) and brine solution (1×20 mL). The organic layer was dried over anhydrous Na₂SO₄, filtered and evaporated under reduced pressure to obtain crude compound. The crude compound was purified by column chromatography (230-400 mesh silica gel) with 20% ethyl acetate in hexane to afford (2) as an off white solid; yield 0.74 g (82%); ¹H NMR (CDCl₃, 300 MHz) δ 8.903 (s, 1H), 7.658 (s, 1H), 7.513-7.462 (m, 4H), 7.407-7.311 (m, 7H), 5,262 (s, 2H), 5.236 (s, 2H).

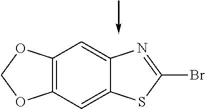

### 6-bromo-[1,3]dioxolo[4′,5′:4,5]benzo[1,2-d]thiazole (3)

To an oven dried Schlenk flask, cooled under argon, was added 2 (1 mmol) and the flask was flushed with argon. 1 ml of DMF was added followed by CBr₄ (1.5 mmol) and K₃PO₄ (3 mmol). The flask was then immersed in a preheated oil bath at 120° C. The reaction mixture was stirred at this temperature for 5 hrs. The reaction mixture was cooled to room temperature, quenched with water, and the resulting mixture was extracted with ethyl acetate three times. The organic layer was collected and dried over anhydrous sodium sulfate. The solvent was evaporated in vacuum to obtain crude product which was then subjected to flash chromatography on silica gel to yield (20%) pure product (3).

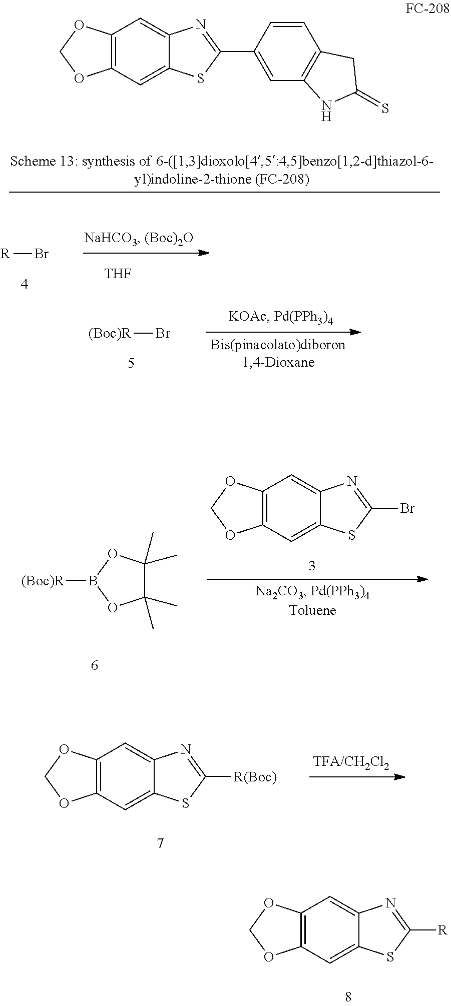

| R group | Yield (5R) | Yield (6R) | Yield (7R) | Yield (8R) |
|---|---|---|---|---|
| 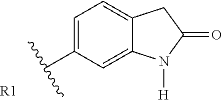 | 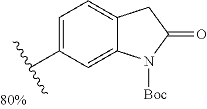 [SMILES: *c1ccc2c(c1)N(C(=O)OC(C)(C)C)C(=O)C2] | 74% | 57% (FC-207) | 95% (FC-206) |

To a stirred mixture of 4 (1 eq) and NaHCO₃ (10 eq) in THF (12 ml) was added (Boc)₂O (2 eq) at room temperature under nitrogen. The resulting mixture was heated to reflux for 3 h. After cooling, the mixture was filtered through Celite and washed with THF. The filtrate was concentrated in vacuo and purified by column chromatography to afford pure product 5. To the solution of 5 (1 eq) in 1,4-dioxane (10 ml), was added KOAc (2 eq), Pd(dppf)Cl₂ (0.1 mol %) and Bis (pinacolato)diboron (1.5 eq) and the reaction mixture was heated to 90° C. for 18 h. The reaction mixture was then cooled and concentrated. The residue was purified by column chromatography to obtain 6. In an oven dried Schlenk flask, under argon, 6 (1.2 eq) and 3 (1 eq), were dissolved in toluene (15 ml). The reaction mixture was degassed and filled with argon three times. Pd(PPh₃)₄ (0.1 mol %) was added to the mixture and it was again degassed and filled with argon three times. A degassed solution of Na₂CO₃ (1.25 g in 5 ml water) was added to the reaction mixture and it was heated to reflux for 16 h. The reaction mixture was cooled, quenched with water and then extracted with ethyl acetate three times. The organic layers were combined and dried over Na₂SO₄. The solvent was evaporated under vacuum to afford crude product which was further purified by column chromatography to give pure product 7. In a flask 7 was dissolved in 1 ml DCM and TFA was added to the solution slowly. The reaction was stirred for 30 min and the reaction progress was monitored by TLC. The solvent was evaporated and the solid was washed with methanol to afford pure product 8.

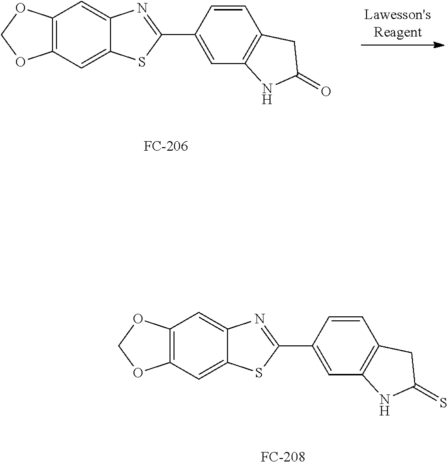 [SMILES: O=C1Cc2ccc(-c3nc4cc5c(cc4s3)OCO5)cc2N1 ; S=C1Cc2ccc(-c3nc4cc5c(cc4s3)OCO5)cc2N1]

In an oven dried flask FC-206 (20 mg) was dissolved in 2 mL of anhydrous toluene followed by treatment with Lawesson's reagent (16 mg). The reaction mixture was heated first at 60° C. for 30 min then the temperature was increased to 120° C. and refluxed for another 2 h or until the disappearance of the starting material. Upon completion, the excess solvent was evaporated under vacuum; the crude mixture was dissolved in EtOAc; the organic layer was washed twice with 5% bleach solution, followed by NaHCO₃ and brine. The organic layer was dried over Na₂SO₄, filtered, and evaporated under vacuum. The crude mixture was washed several times with methanol to afford FC-208 (20 mg).

A skilled person could make compounds in accordance with the present disclosure, including, for example, all those described above and all of those depicted in Table II below, following the procedures described above and substituting the appropriate starting materials as may be relevant for a given example.

Kinase activity was assayed as follows:

### Kinase Assays

### Radiolabelled Kinase Assay (IC₅₀ Determination)

Protein kinase assays using radiolabeled [γ-³²P] ATP remains the “gold standard” against which the performance of nonradioactive assay techniques is measured. In this assay, the activity of a protein kinase is measured using an appropriate acceptor peptide or protein substrate. The assay method involves the use of P81 phosphocellulose paper squares to capture the phosphorylated peptides or proteins resulting from the protein kinase reaction. The activity of DYRK1A was measured by its ability to phosphorylate the peptide termed Woodtide that is derived from the substrate FKHR (Forkhead in rhabdomyosarcoma) at Ser329. Two lysine residues are added at the N-terminus of the peptide to facilitate its binding to the phosphocellulose paper (KKISGRLSPIMTEQ). Assays were carried out at 30° C. for 10 mins using 25 μM Woodtide in 50 mM HEPES pH 7.5, 50 mM MgCl₂, 50 mM DTT and 0.1 mM [γ-³²P] ATP (106 c.p.m./nmol). For determination of IC₅₀ of the compounds, increasing concentrations of the inhibitors (3 nM -10 μM) were incubated in the presence of DYRK1A and Woodtide and its kinase activity was measured. The readings were taken using a scintillation counter and data was analyzed using GraphPad Prism 8. One unit of enzyme activity was the amount that catalyzed the phosphorylation of 1 nmol of Woodtide in 1 min.

### ADP-Glo Assay—Promega (Compound Screening)

The ADP-GloTM Kinase Assay kit from Promega was used to screen the compounds before determination of their IC₅₀ using the radiolabeled kinase assay. This assay kit is a luminescent ADP detection assay that provides a universal, homogeneous, high-throughput screening method to measure kinase activity. It quantifies the amount of ADP produced during a kinase reaction. The ADP-GloTM Kinase Assay was performed in a 384-multiwell plate using the manufacturer's protocol. Assays were carried out at room temperature for 10 mins using 25 μM Woodtide and 25 μM ATP in 50 mM HEPES pH 7.5, 50 mM MgCl₂, 50 mM DTT. The data was analyzed using GraphPad Prism 8.

Examples of compounds of Formula I that inhibit DYRK1A or PIM1 were identified, with the following IC₅₀ values:

TABLE II
IC₅₀ values for DYRK1A and PIM1

| | | DYRK1A | PIM1 |
|---|---|---|---|
| Compound | Formula | IC₅₀ (nm) | IC₅₀ (nm) |
| FC-2 | 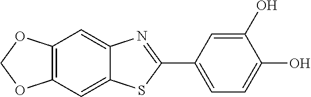 [SMILES: Oc1ccc(-c2nc3cc4c(cc3s2)OCO4)cc1O] | 19 | Micromolar inhibition |
| FC-3 | 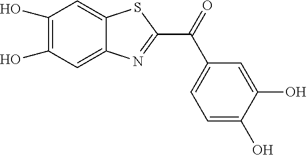 [SMILES: O=C(c1ccc(O)c(O)c1)c1nc2cc(O)c(O)cc2s1] | 53 | Micromolar inhibition |
| FC-4 | 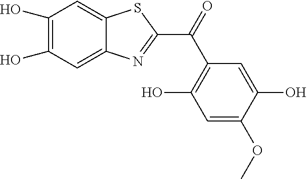 [SMILES: COc1cc(O)c(C(=O)c2nc3cc(O)c(O)cc3s2)cc1O] | 87 | 174 |
| FC-5 | 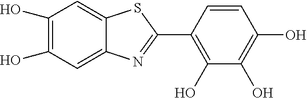 [SMILES: Oc1cc2nc(-c3ccc(O)c(O)c3O)sc2cc1O] | 95 | 144 |
| FC-6 | 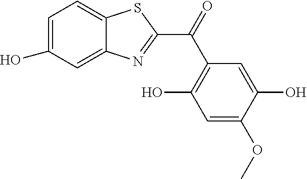 [SMILES: COc1cc(O)c(C(=O)c2nc3cc(O)ccc3s2)cc1O] | 180 | 552.3 |
| FC-7 | 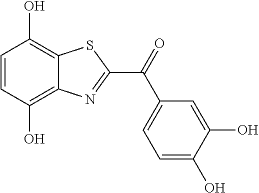 [SMILES: O=C(c1ccc(O)c(O)c1)c1nc2c(O)ccc(O)c2s1] | 229 | Micromolar inhibition |
| FC-7A | 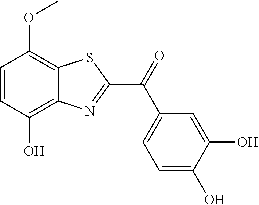 [SMILES: COc1ccc(O)c2nc(C(=O)c3ccc(O)c(O)c3)sc12] | 560 | NA |
| FC-8 | 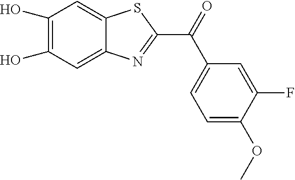 [SMILES: COc1ccc(C(=O)c2nc3cc(O)c(O)cc3s2)cc1F] | 238 | 317 |
| FC-10 | 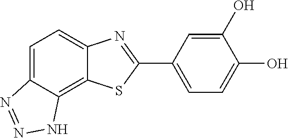 [SMILES: Oc1ccc(-c2nc3ccc4nn[nH]c4c3s2)cc1O] | 110 | NA |
| FC-14 | 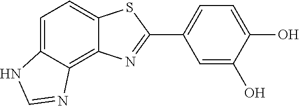 [SMILES: Oc1ccc(-c2nc3c(ccc4[nH]cnc43)s2)cc1O] | 400 | NA |
| FC-63 | 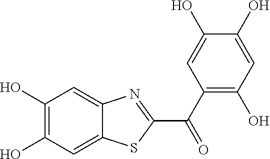 [SMILES: O=C(c1nc2cc(O)c(O)cc2s1)c1cc(O)c(O)cc1O] | Micromolar inhibition | 238.4 |
| FC-115 | 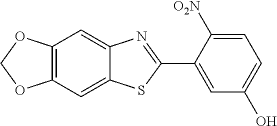 [SMILES: O=[N+]([O-])c1ccc(O)cc1-c1nc2cc3c(cc2s1)OCO3] | 524 | No Inhibition |
| FC-195 | 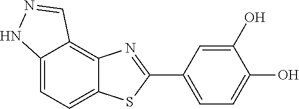 [SMILES: Oc1ccc(-c2nc3c(ccc4[nH]ncc43)s2)cc1O] | 420 | NA |
| FC-196 | 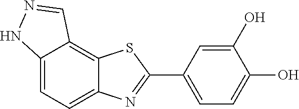 [SMILES: Oc1ccc(-c2nc3ccc4[nH]ncc4c3s2)cc1O] | 500 | NA |
| FC-204 | 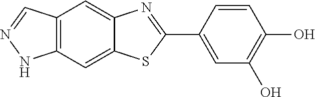 [SMILES: Oc1ccc(-c2nc3cc4cn[nH]c4cc3s2)cc1O] | 57 | NA |
| FC-205 | 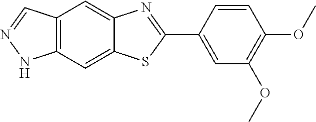 [SMILES: COc1ccc(-c2nc3cc4cn[nH]c4cc3s2)cc1OC] | 206 | NA |
| FC-208 | 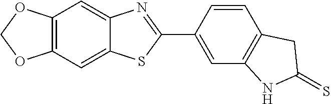 [SMILES: S=C1Cc2ccc(-c3nc4cc5c(cc4s3)OCO5)cc2N1] | 560 | NA |

DYRK1A and PIM1 inhibition are potential targets for anti-cancer treatments. Thus, compounds of Formula I were tested for possible anti-cancer activity, namely antiproliferative effects on a glioblastoma cell line (U87MG) in a neurosphere proliferation assay, and inhibition of metastatic potential on a glioblastoma cell line (U87MG) in an in vitro invasion assay.

### Neurosphere Proliferation Assay

Neurospheres are free-floating 3-D clusters of neural stem cells that are grown in serum-free media supplemented with growth factors. These cells have the capacity to self-renew and differentiate into cell types present within the tumor of origin. They are also responsible for tumor propagation, recurrence and resistance to traditional treatments. Neurosphere assays are commonly used to uncover more relevant brain tumor biology than traditional culture conditions. Several parameters can be assayed including neurosphere number and size. Neurosphere formation remained a significant predictor of clinical outcome in GBM, independent of the Ki67 labeling index. DYRK1A has been shown to regulate the self-renewal capacity of neurospheres. Self-renewal is the ability of a single cell to form a neurosphere.

Two EGFR-dependent GBM cell-lines, U87MG and LN229 were grown under serum free conditions with growth factors to induce the formation of neurospheres. Their stemness was confirmed using a western blot to detect stem cell markers. Both inhibition using FC2 and FC3 as well as knockout of DYRK1A using CRISPR-Cas9 in these cell lines resulted in a significant decrease in neurosphere proliferation which included neurosphere numbers and size (as shown in the dot plots; each dot represents one neurosphere). This reaffirmed the fact that DYRK1A plays a crucial role in regulating neurosphere self-renewal in GBM.

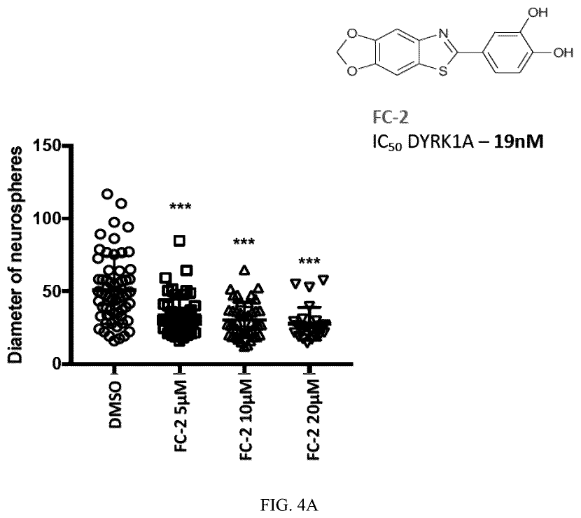

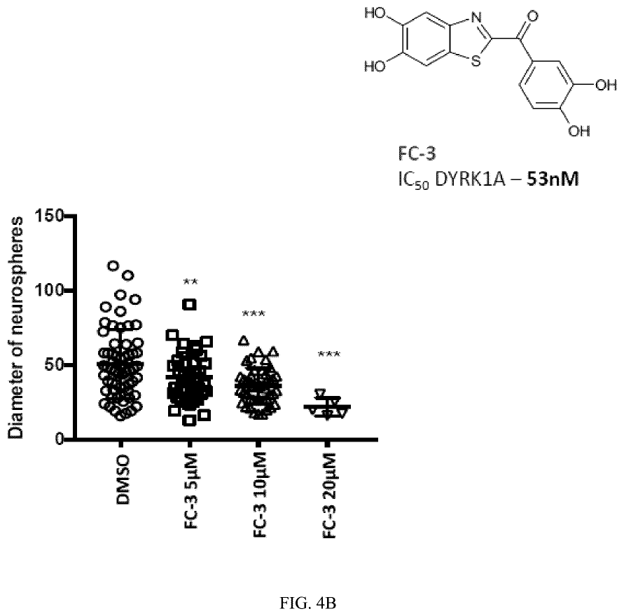

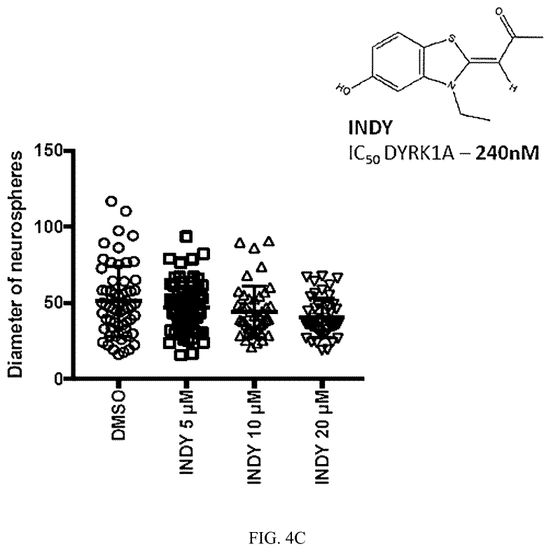

In the neurosphere proliferation assay, FC-2 and FC-3 significantly inhibited proliferation of U87MG cells, assessed by decreased neurosphere diameter, at all concentrations tested (5 μM, 10 μM, and 20 μM) compared to DMSO vehicle control. By contrast, the known DYRK1A inhibitor INDY did not inhibit proliferation of U87MG cells. Results are shown in FIGS. 4A-4C. Compounds of Formula I therefore have an anticancer potential and inhibit proliferation of cells with a cancer phenotype.

### Invasion Assay

The Invasion Assay provides an in vitro system to study cell invasion of malignant and normal cells. Specific applications include assessment of the metastatic potential of tumor cells. Invasion chambers coated with Corning® Matrigel® matrix provide cells with the conditions that allow assessment of their invasive capacity in vitro. Corning Matrigel matrix serves as a reconstituted basement membrane in vitro, occluding the pores of the membrane and blocking non-invasive cells from migrating through the membrane. In contrast, invasive cells (malignant and non-malignant) secrete proteases that enzymatically degrade the Corning Matrigel matrix and enable invasion through the membrane pores.

In order to assess whether DYRK1A plays a role in cell invasion, we performed an invasion assay using the two GBM cell lines U87MG and LN229. We found that both inhibition using FC2 and FC3 as well as knockout of DYRK1A impaired the ability of the cells to invade the Matrigel chamber. This indicates that DYRK1A might be important in mediating invasion in GBM cells. Inhibition of DYRK1A using FC2 and FC3 can also decrease the metastatic potential of GBM cells, thus making them good therapeutic candidates for EGFR-dependent GBM.

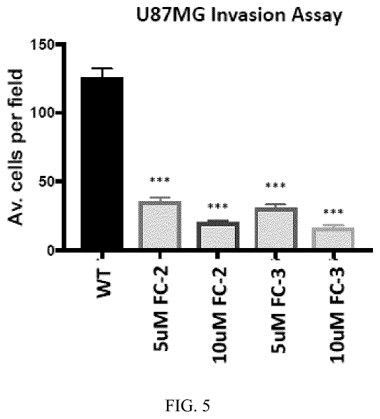

In the cell invasion assay, FC-2 and FC-3, at the tested concentrations of 5 μM and 10 μM, significantly inhibited cell invasion, relative to DMSO vehicle. Results are shown in FIG. 5.

### Possible Basis of Specificity of FC2 and FC3 to DYRK Kinases

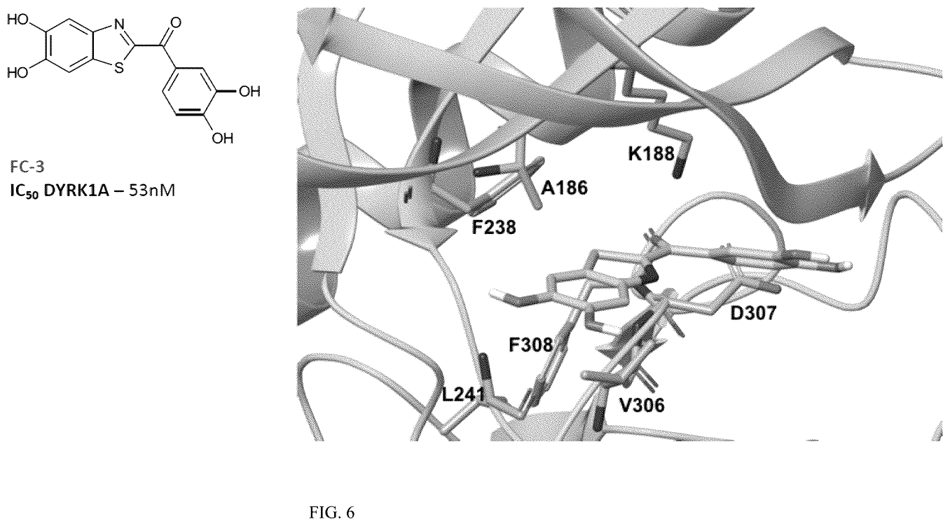

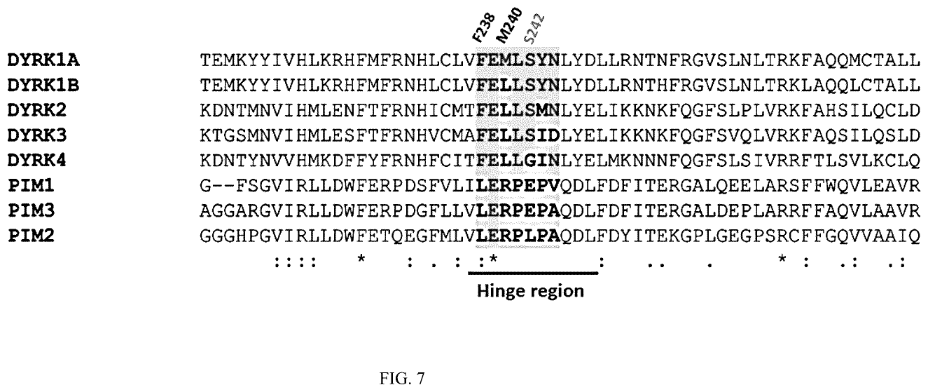

On the basis of the crystal structure of DYRK1A with FC3 (FIG. 6), modeling studies of DYRK1A with both FC2 and FC3 and in-vitro ATP competitive assays, both FC2 and FC3 are ATP-competitive inhibitors that bind to the ATP binding site of DYRK1A. Furthermore, when FC2 and FC3 were tested against a panel of 140 different kinases, they were found to be very specific to the DYRK family of kinases. In order to investigate the basis of specificity of FC2 and FC3 towards DYRKs over PIMs, sequences of DYRKs and PIMs were aligned. Interestingly, the hinge region of DYRK1A that precedes the ATP-binding pocket showed the highest degree of variation from PIMs. See FIG. 7.

Kinase domains contain a gatekeeper residue that partially or fully blocks a hydrophobic region deep in the ATP binding pocket. The gatekeeper residue contributes to the selectivity of kinases for small molecule inhibitors. Based on the DYRK1A crystal structure with FC3 and sequence alignment, the gatekeeper residue in DYRK1A is Phe238 which is replaced by a smaller Leu210 in case of PIM1 and other PIM kinases. Similarly, Met240, two residues after the gatekeeper residue was very unique to DYRK1A. Another interesting residue was Ser241 which was conserved among DYRKs. Point mutants of the gatekeeper residue (F238L), Met240 (M240R), Ser240 (S240A) and a double mutant (F238LM240R) were created to evaluate if the point mutants show resistance towards FC2 and FC3.

In-vitro radiolabeled kinase assays were used to determine the IC₅₀s of FC2 and FC3 against the DYRK1A point mutants. There was no change in the Km or Vmax of the point mutants of DYRK1A and their activity was similar to wild-type DYRK1A. Interestingly we found that the IC₅₀s of FC2 and FC3 were significantly higher for the point mutants F238L, M240R, S242A (only for FC3) and even higher for the double mutant (F238LM240R). This indicated that the hinge region that includes the gatekeeper might be critical for conferring resistance to DYRKs.

Furthermore, the effect of the point mutants of DYRK1A were tested in the neurosphere invasion assays in the glioblastoma cell-line U87MG. For both the assays, DYRK1A knockouts generated using CRISPR-Cas9 were rescued with DYRK1A point mutant F238LM240R. The rescue knockouts were then treated with FC2 and FC3 to test their effect on neurosphere proliferation and invasion. The DYRK1A point mutants treated with FC2 and FC3 did not show a decrease in neurosphere proliferation or invasion when compared to DYRK1A wild-type treated-cells.

Thus, based on both in-vitro and cell-based assays, a feature conferring specificity to FC2 and FC3 towards DYRKs over other kinases may include the hinge region of DYRK1A.

Although examples have been depicted and described in detail herein, it will be apparent to those skilled in the relevant art that various modifications, additions, substitutions, and the like can be made without departing from the spirit of the present disclosure and these are therefore considered to be within the scope of the present disclosure as defined in the claims that follow.

What is claimed is:

1. A compound, comprising Formula I: 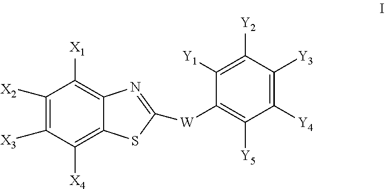
or a pharmaceutically acceptable salt thereof, wherein W is a direct bond or an optionally substituted C, and
if W is a direct bond, then X₁, X₂, X₃, and X₄ are each independently H, OH, or an electrophile, and optionally one pair selected from X₁ and X₂, X₂ and X₃, and X₃ and X₄ forms a five-membered ring including one or more heteroatom, wherein the one or more heteroatom is selected from O, N, and S, and the five-membered ring is optionally substituted with a ═C, an ═S, or an electrophile, and
Y₁, Y₂, Y₃, Y₄ and Y₅ are each independently selected from H, CH₃, OH, O—CH₃, NO₂, and an electrophile, and optionally Y₂ and Y₃ form a five-membered ring including one or more heteroatom, wherein the one or more heteroatom is selected from O, N, and S, and the five-membered ring is optionally substituted with a ═C, ═S, or an electrophile,
with the caveats that
(a) if the pair X₂ and X₃ together form a dioxolane then optionally Y₂ and Y₃ form a five-membered ring including one or more heteroatom only if Y₂ and Y₃ form a pyrroline optionally substituted with a ═C, ═S, or an electrophile, and no more than one of Y₁, Y₂, Y₃, Y₄ and Y₅ is OH,
(b) if one or more of Y₁, Y₂, Y₃, Y₄ and Y₅ is O—CH₃, then X₂ and X₃ form a five-membered ring including one or more heteroatom, wherein the one or more heteroatom is selected from N and S, and the five-membered ring is optionally substituted with a ═C, ═S, or an electrophile,
(c) if X₃ is OH and one or both of Y₃ and Y₄ are OH then Y₅ is OH; and
(d) if W is an optionally substituted C, then
(i) at least one of X₁, X₂, X₃, X₄ is independently selected from OH, O—CH₃, and a halogen, at least one of Y₁, Y₂, Y₃, Y₄, and Y₅ is independently selected from OH, O—CH₃, and a halogen, no more than five of X₁, X₂, X₃, X₄, Y₁, Y₂, Y₃, Y₄, and Y₅ is H, and no more than one of X₁, X₂, X₃, X₄, Y₁, Y₂, Y₃, Y₄, and Y₅, is O—CH₃, or
(ii) X₁, X₄, Y₁, Y₄, and Y₅ are hydrogen, X₂ and X₃ are OH or together form a dioxolane, and Y₂ and Y₃ are OH or together form a dioxolane.

2. The compound of claim 1, wherein W is an optionally substituted C.

3. The compound of claim 2, wherein W comprises —C(═O)—.

4. The compound of claim 2 or 3, wherein at least one of X₁, X₂, X₃, X₄ is independently selected from OH, O—CH₃, and a halogen, at least one of Y₁, Y₂, Y₃, Y₄, and Y₅ is independently selected from OH, O—CH₃, and a halogen, no more than five of X₁, X₂, X₃, X₄, Y₁, Y₂, Y₃, Y₄, and Y₅ is H, and no more than one of X₁, X₂, X₃, X₄, Y₁, Y₂, Y₃, Y₄, and Y₅, is O—CH₃.

5. The compound of claim 2 or 3, wherein X₁, X₄, Y₁, Y₄, and Y₅ are hydrogen, X₂ and X₃ are OH or together form a dioxolane, and Y₂ and Y₃ are OH or together form a dioxolane.

6. The compound of claim 1, wherein the compound is selected from

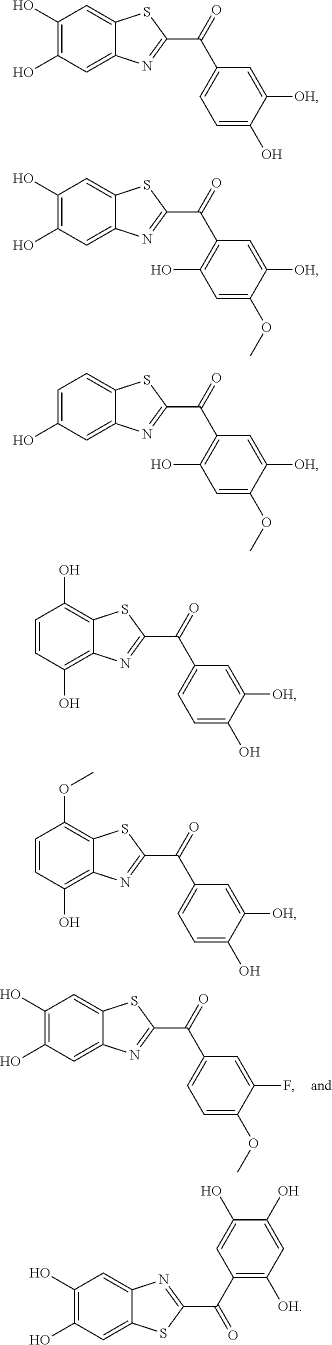

7. The compound of claim 1, comprising Formula Ia: 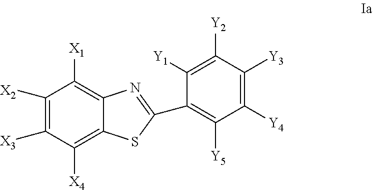
or a pharmaceutically acceptable salt thereof.

8. The compound of claim 7, wherein X₁ and X₂, or X₃ and X₄, form an imidazole or a triazole.

9. The compound of claim 7, wherein X₂ and X₃ form a five-membered ring selected from a pyrrole, a dioxolane, a pyrazole, an imidazole, and a triazole.

10. The compound of claim 7, wherein X₂ and X₃ form a five-membered ring comprising a substitution, the five-membered ring is selected from a pyrroline and a tetrahydrofuran, and the substitution is selected from ═O, ═S, and an electrophile.

11. The compound of claim 7, wherein X₂ and X₃ form a thiazole comprising a substitution, and the substitution is selected from ═O, ═S, and an electrophile.

12. The compound of claim 7, wherein X₂ and X₃ form a dioxolane.

13. The compound of claim 12, wherein Y₂ and Y₃ form a five-membered ring.

14. The compound of claim 13, wherein Y₂ and Y₃ form a furan, a pyrrole, or a thiophene.

15. The compound of claim 7, wherein Y₂ and Y₃ form a five-membered ring comprising a substitution, wherein the five-membered ring is selected from a pyrroline and a thiazole, and the substitution is selected from ═O, ═S, and an electrophile.

16. The compound of claim 7, wherein the compound is selected from

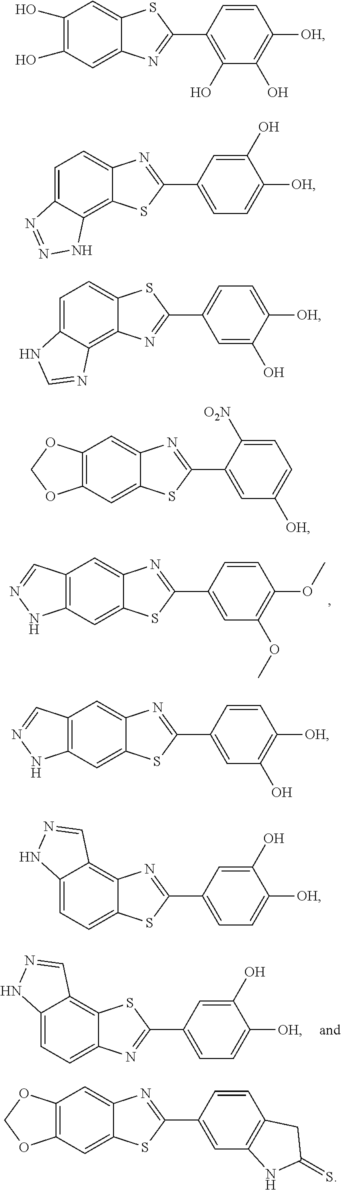

17. The compound of any one of claims 1 through 5 and 7 through 15, wherein the electrophile is selected from 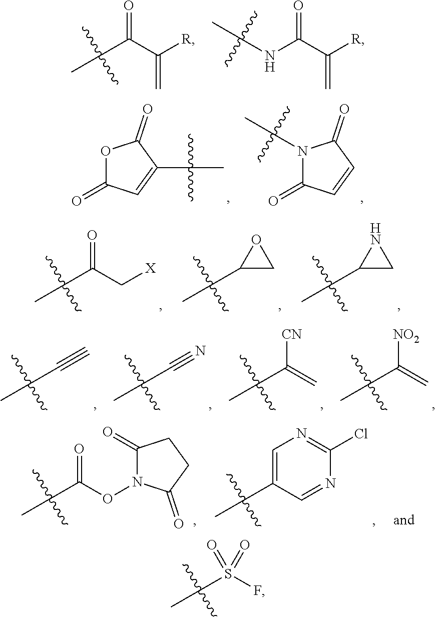
wherein R═H or any alkyl or any carbonyl, and X=any halogen, ether, amine, thiol, or thioether.

18. The compound of any one of claims 1 through 5 and 7 through 15, wherein the compound is not substituted with an electrophile.

19. A pharmaceutical composition, comprising the compound of any one of claims 1 through 18 and a pharmaceutically acceptable excipient.

20. A method, comprising administering a pharmaceutical composition of claim 19 to a subject, wherein the subject is diagnosed with or at risk of developing cancer.

21. The method of claim 20, further comprising administering an epidermal growth factor receptor (EGFR) inhibitor in combination with the pharmaceutical composition, or wherein the pharmaceutical composition further comprises the EGFR inhibitor.

22. The method of claim 21, wherein the EGFR inhibitor comprises AZD9291.

23. The method of any one of claims 20 through 22, wherein the cancer is selected from acute megakaryoplastic leukemia, glioblastoma, and non-small cell lung cancer.

24. A method, comprising treating an impairment in a subject, wherein the impairment includes a cognitive impairment or an affective impairment, and the treating comprises administering the pharmaceutical composition of claim 19 to the subject.

25. The method of claim 26, wherein the subject is diagnosed with Down syndrome.

26. The method of claim 26, wherein the subject is diagnosed with or at risk for developing Alzheimer's disease.

27. A method, comprising administering the pharmaceutical composition of claim 19 to a subject, wherein the subject is diagnosed with or at risk for developing Alzheimer's disease.

28. A method, comprising administering a pharmaceutical composition of claim 19 to a subject, wherein the subject is diagnosed with Down syndrome.

29. A compound, comprising Formula I: 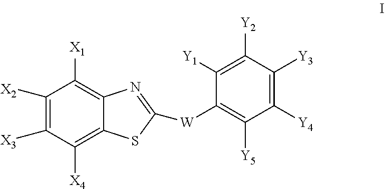
or a pharmaceutically acceptable salt thereof, wherein
W is an optionally substituted C,
at least one of X₁, X₂, X₃, X₄ is independently selected from OH, O—CH₃, and a halogen,
Y₁, Y₂, Y₃, Y₄ and Y₅ are each independently selected from H, CH₃, OH, O—CH₃, NO₂, and an electrophile, at least one of Y₁, Y₂, Y₃, Y₄, and Y₅ is independently selected from OH and O—CH₃, and a halogen, and optionally Y₂ and Y₃ form a five-membered ring including one or more heteroatom, wherein the one or more heteroatom is selected from O, N, and S, and the five-membered ring is optionally substituted with a ═C, ═S, or an electrophile, and
no more than five of X₁, X₂, X₃, X₄, Y₁, Y₂, Y₃, Y₄, and Y₅ is H, and no more than one of X₁, X₂, X₃, X₄, Y₁, Y₂, Y₃, Y₄, and Y₅, is O—CH₃.

30. The compound of claim 29, wherein W comprises —C(═O)—.

31. The compound of claim 29 or 30, wherein the compound is selected from

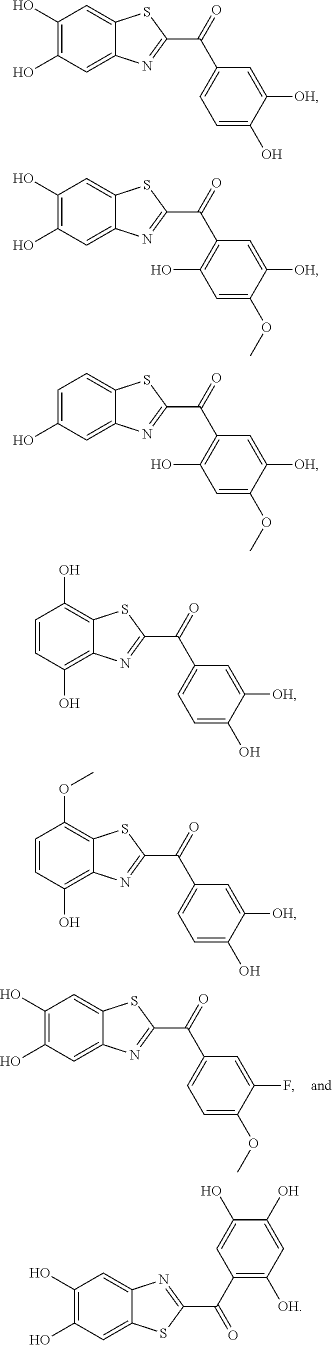

32. The compound of any one of claims 29 through 31, wherein the electrophile is selected from 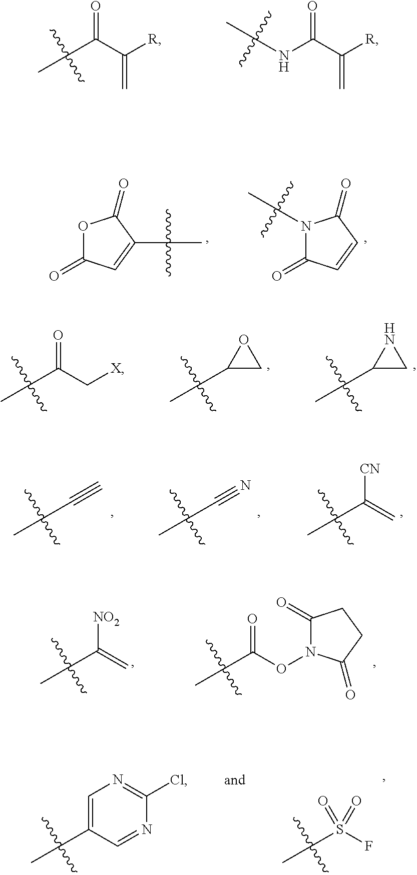
wherein R═H or any alkyl or any carbonyl, and X=any halogen, ether, amine, thiol, or thioether.

33. The compound of any one of claims 29 through 31, wherein the compound is not substituted with an electrophile.

34. A pharmaceutical composition, comprising the compound of any one of claims 29 through 33 and a pharmaceutically acceptable excipient.

35. A compound, comprising Formula I: 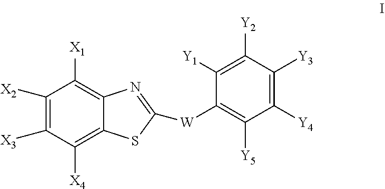
or a pharmaceutically acceptable salt thereof, wherein
W is an optionally substituted C, and
X₁, X₄, Y₁, Y₄, and Y₅ are hydrogen, X₂ and X₃ are OH or together form a dioxolane, and Y₂ and Y₃ are OH or together form a dioxolane.

36. The compound of claim 35, wherein W comprises —C(═O)—.

37. The compound of claim 35 or 36, wherein the compound is

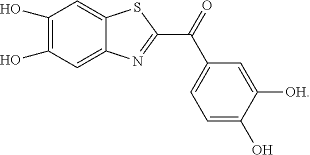 [SMILES: O=C(c1ccc(O)c(O)c1)c1nc2cc(O)c(O)cc2s1]

38. A pharmaceutical composition, comprising the compound of any one of claims 35 through 37 and a pharmaceutically acceptable excipient.

39. A compound, comprising Formula Ia: 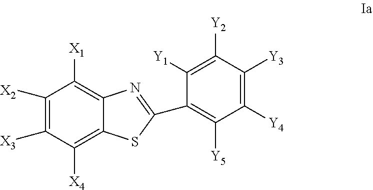
or a pharmaceutically acceptable salt thereof, wherein
X₁, X₂, X₃, and X₄ are each independently H, OH, or an electrophile, and optionally one pair selected from X₁ and X₂, X₂ and X₃, and X₃ and X₄ forms a five-membered ring including one or more heteroatom, wherein the one or more heteroatom is selected from O, N, and S, and the five-membered ring is optionally substituted with a ═C, an ═S, or an electrophile, and
Y₁, Y₂, Y₃, Y₄ and Y₅ are each independently selected from H, CH₃, OH, O—CH₃, NO₂, and an electrophile, and optionally Y₂ and Y₃ form a five-membered ring including one or more heteroatom, wherein the one or more heteroatom is selected from O, N, and S, and the five-membered ring is optionally substituted with a ═C, ═S, or an electrophile,
with the caveats that
(a) if the pair X₂ and X₃ together form a dioxolane then optionally Y₂ and Y₃ form a pyrroline optionally substituted with a ═C, ═S, or an electrophile, and no more than one of Y₁, Y₂, Y₃, Y₄ and Y₅ is OH,
(b) if one or more of Y₁, Y₂, Y₃, Y₄ and Y₅ is O—CH₃, then X₂ and X₃ form a five-membered ring including one or more heteroatom, wherein the one or more heteroatom is selected from N and S, and the five-membered ring is optionally substituted with a ═C, ═S, or an electrophile, and
(c) if X₃ is OH and one or both of Y₃ and Y₄ are OH then Y₅ is OH.

40. The compound of claim 39, wherein X₁ and X₂, or X₃ and X₄, form an imidazole or a triazole.

41. The compound of claim 39, wherein X₂ and X₃ form a five-membered ring selected from a pyrrole, a dioxolane, a pyrazole, an imidazole, and a triazole.

42. The compound of claim 39, wherein X₂ and X₃ form a five-membered ring comprising a substitution, the five-membered ring is selected from a pyrroline and a tetrahydrofuran, and the substitution is selected from ═O, ═S, and an electrophile.

43. The compound of claim 40, wherein X₂ and X₃ form a thiazole comprising a substitution, and the substitution is selected from ═O, ═S, and an electrophile.

44. The compound of claim 39, wherein X₂ and X₃ form a dioxolane.

45. The compound of claim 44, wherein Y₂ and Y₃ form a five-membered ring.

46. The compound of claim 45, wherein Y₂ and Y₃ form a furan, a pyrrole, or a thiophene.

47. The compound of claim 39, wherein Y₂ and Y₃ form a five-membered ring comprising a substitution, wherein the five-membered ring is selected from a pyrroline and a thiazole, and the substitution is selected from ═O, ═S, and an electrophile.

48. The compound of claim 39, wherein the compound is selected from

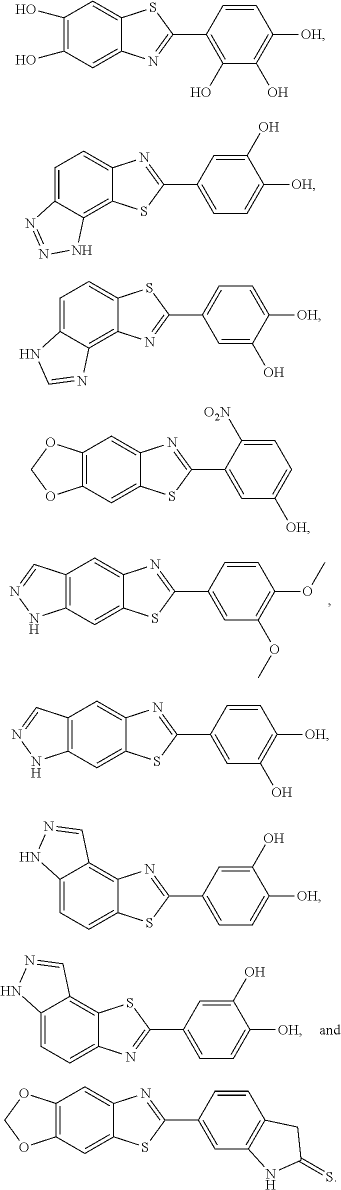

49. The compound of any one of claims 39 through 48, wherein the electrophile is selected from 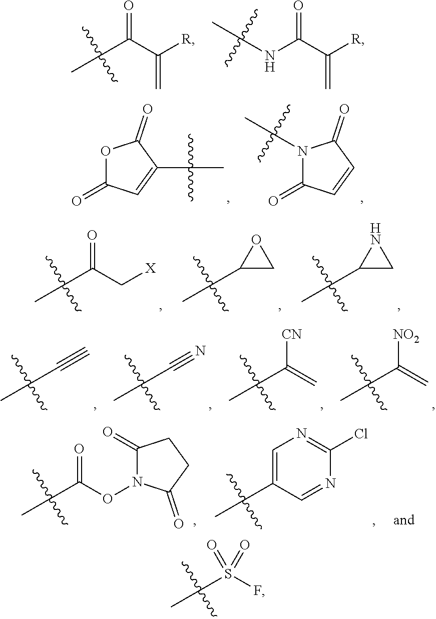
wherein R═H or any alkyl or any carbonyl, and X=any halogen, ether, amine, thiol, or thioether.

50. The compound of any one of claims 39 through 48, wherein the compound is not substituted with an electrophile.

51. A pharmaceutical composition, comprising the compound of any one of claims 39 through 50 and a pharmaceutically acceptable excipient.

52. A compound, comprising Formula Ia: 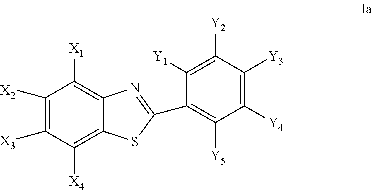
or a pharmaceutically acceptable salt thereof, wherein
X₁, X₂, X₃, and X₄ are each independently H, OH, or an electrophile, and optionally one pair selected from X₁ and X₂, X₂ and X₃, and X₃ and X₄ forms a five-membered ring including one or more heteroatom, wherein the one or more heteroatom is selected from O, N, and S, and the five-membered ring is not a dioxolane and is optionally substituted with a ═C, an ═S, or an electrophile, and
Y₁, Y₂, Y₃, Y₄ and Y₅ are each independently selected from H, CH₃, OH, O—CH₃, NO₂, and an electrophile, and optionally Y₂ and Y₃ form a five-membered ring including one or more heteroatom, wherein the one or more heteroatom is selected from O, N, and S, and the five-membered ring is optionally substituted with a ═C, ═S, or an electrophile,
with the caveats that
(a) if one or more of Y₁, Y₂, Y₃, Y₄ and Y₅ is O—CH₃, then X₂ and X₃ form a five-membered ring including one or more heteroatom, wherein the one or more heteroatom is selected from N and S, and the five-membered ring is optionally substituted with a ═C, ═S, or an electrophile, and
(b) if X₃ is OH and one or both of Y₃ and Y₄ are OH then Y₅ is OH.

53. The compound of claim 52, wherein X₁ and X₂, or X₃ and X₄, form an imidazole or a triazole.

54. The compound of claim 52, wherein X₂ and X₃ form a five-membered ring selected from a pyrrole, a dioxolane, a pyrazole, an imidazole, and a triazole.

55. The compound of claim 52, wherein X₂ and X₃ form a five-membered ring comprising a substitution, the five-membered ring is selected from a pyrroline and a tetrahydrofuran, and the substitution is selected from ═O, ═S, and an electrophile.

56. The compound of claim 52, wherein X₂ and X₃ form a thiazole comprising a substitution, and the substitution is selected from ═O, ═S, and an electrophile.

57. The compound of claim 52, wherein X₂ and X₃ form a dioxolane.

58. The compound of claim 57, wherein Y₂ and Y₃ form a five-membered ring.

59. The compound of claim 58, wherein Y₂ and Y₃ form a furan, a pyrrole, or a thiophene.

60. The compound of claim 52, wherein Y₂ and Y₃ form a five-membered ring comprising a substitution, wherein the five-membered ring is selected from a pyrroline and a thiazole, and the substitution is selected from ═O, ═S, and an electrophile.

61. The compound of claim 52, wherein the compound is selected from

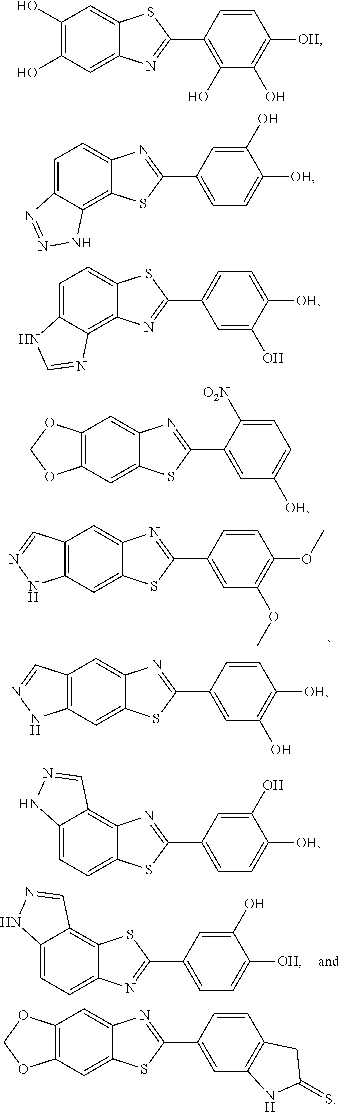

62. The compound of any one of claims 52 through 61, wherein the electrophile is selected from 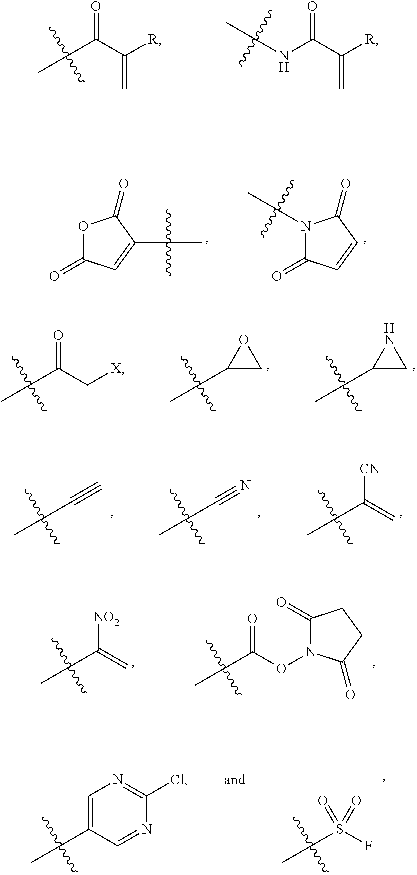
wherein R═H or any alkyl or any carbonyl, and X=any halogen, ether, amine, thiol, or thioether.

63. The compound of any one of claims 52 through 61, wherein the compound is not substituted with an electrophile.

64. A pharmaceutical composition, comprising the compound of any one of claims 52 through 63 and a pharmaceutically acceptable excipient.

65. A compound, comprising Formula Ia: 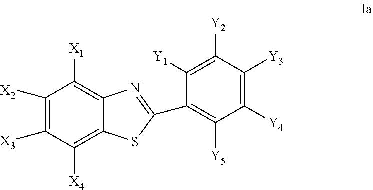
or a pharmaceutically acceptable salt thereof, wherein
X₁, X₂, X₃, and X₄ are each independently H, OH, or an electrophile, and optionally one pair selected from X₁ and X₂, X₂ and X₃, and X₃ and X₄ forms a five-membered ring including one or more heteroatom, wherein the one or more heteroatom is selected from O, N, and S, and the five-membered ring is optionally substituted with a ═C, an ═S, or an electrophile, and
Y₁, Y₂, Y₃, Y₄ and Y₅ are each independently selected from H, CH₃, OH, NO₂, and an electrophile, and optionally Y₂ and Y₃ form a five-membered ring including one or more heteroatom, wherein the one or more heteroatom is selected from O, N, and S, and the five-membered ring is optionally substituted with a ═C, ═S, or an electrophile,
with the caveats that
(a) if the pair X₂ and X₃ together form a dioxolane then optionally Y₂ and Y₃ form a five-membered ring including one or more heteroatom only if Y₂ and Y₃ form a pyrroline optionally substituted with a ═C, ═S, or an electrophile, and no more than one of Y₁, Y₂, Y₃, Y₄ and Y₅ is OH, and.
(b) if X₃ is OH and one or both of Y₃ and Y₄ are OH then Y₅ is OH

66. The compound of claim 65, wherein X₁ and X₂, or X₃ and X₄, form an imidazole or a triazole.

67. The compound of claim 65, wherein X₂ and X₃ form a five-membered ring selected from a pyrrole, a dioxolane, a pyrazole, an imidazole, and a triazole.

68. The compound of claim 65, wherein X₂ and X₃ form a five-membered ring comprising a substitution, the five-membered ring is selected from a pyrroline and a tetrahydrofuran, and the substitution is selected from ═O, ═S, and an electrophile.

69. The compound of claim 65, wherein X₂ and X₃ form a thiazole comprising a substitution, and the substitution is selected from ═O, ═S, and an electrophile.

70. The compound of claim 65, wherein X₂ and X₃ form a dioxolane.

71. The compound of claim 70, wherein Y₂ and Y₃ form a five-membered ring.

72. The compound of claim 71, wherein Y₂ and Y₃ form a furan, a pyrrole, or a thiophene.

73. The compound of claim 65, wherein Y₂ and Y₃ form a five-membered ring comprising a substitution, wherein the five-membered ring is selected from a pyrroline and a thiazole, and the substitution is selected from ═O, ═S, and an electrophile.

74. The compound of claim 65, wherein the compound is selected from

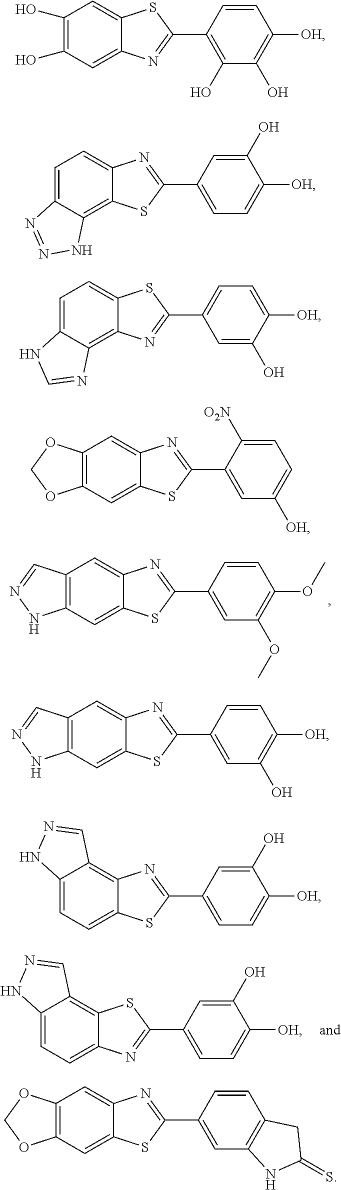

75. The compound of any one of claims 65 through 74, wherein the electrophile is selected from 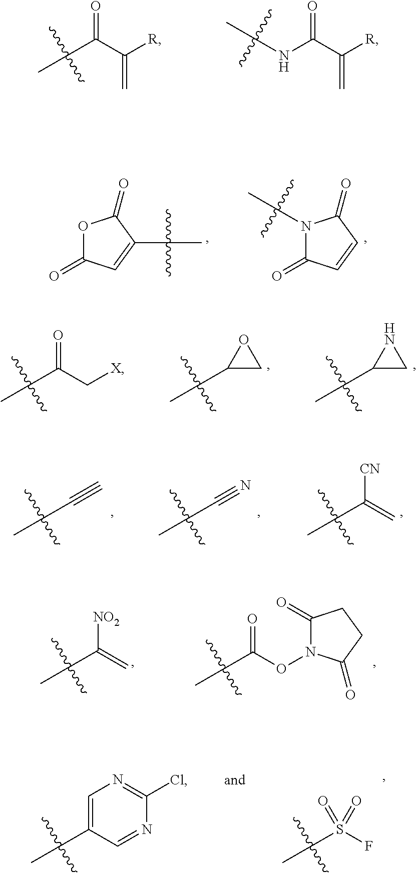
wherein R═H or any alkyl or any carbonyl, and X=any halogen, ether, amine, thiol, or thioether.

76. The compound of any one of claims 65 through 74, wherein the compound is not substituted with an electrophile.

77. A pharmaceutical composition, comprising the compound of any one of claims 65 through 76 and a pharmaceutically acceptable excipient.

78. A method, comprising administering the compound or pharmaceutical acceptable salt thereof of any one of claims 29 through 33, 35 through 37, 39 through 50, 52 through 63, 65 through 76, or pharmaceutical composition of any one of claim 34, 38, 51, 64, or 77 to a subject, wherein the subject is diagnosed with or at risk of developing cancer.

79. The method of claim 78, further comprising administering an epidermal growth factor receptor (EGFR) inhibitor in combination with the pharmaceutical composition, or wherein the pharmaceutical composition further comprises the EGFR inhibitor.

80. The method of claim 79, wherein the EGFR inhibitor comprises AZD9291.

81. The method of any one of claims 78 through 80, wherein the cancer is selected from acute megakaryoplastic leukemia, glioblastoma, and non-small cell lung cancer.

82. A method, comprising treating an impairment in a subject, wherein the impairment includes a cognitive impairment or an affective impairment, and the treating comprises administering the compound or pharmaceutical acceptable salt thereof of any one of claims 29 through 33, 35 through 37, 39 through 50, 52 through 63, 65 through 76, or pharmaceutical composition of any one of claim 34, 38, 51, 64, or 77 to the subject.

83. The method of claim 82, wherein the subject is diagnosed with Down syndrome.

84. The method of claim 82, wherein the subject is diagnosed with or at risk for developing Alzheimer's disease.

85. A method, comprising administering the compound or pharmaceutical acceptable salt thereof of any one of claims 29 through 33, 35 through 37, 39 through 50, 52 through 63, 65 through 76, or pharmaceutical composition of any one of claim 34, 38, 51, 64, or 77 to a subject, wherein the subject is diagnosed with or at risk for developing Alzheimer's disease.

86. A method, comprising administering the compound or pharmaceutical acceptable salt thereof of any one of claims 29 through 33, 35 through 37, 39 through 50, 52 through 63, 65 through 76, or pharmaceutical composition of any one of claim 34, 38, 51, 64, or 77 to a subject, wherein the subject is diagnosed with Down syndrome.
